(19) United States
(12) Patent Application Publication
(10) Pub. No.: US 2017/0296625 A1
(43) Pub. Date: Oct. 19, 2017

(54) THERAPEUTIC ANGIOGENESIS FOR WOUND HEALING

(71) Applicant: CARDIOVASCULAR BIOTHERAPEUTICS, INC., Las Vegas, NV (US)

(72) Inventors: Vance GARDNER, Irvine, CA (US); Kenneth THOMAS, Chatham, NJ (US); John JACOBS, Berkeley, CA (US); Mickael FLAA, Las Vegas, NV (US)

(21) Appl. No.: 15/641,658

(22) Filed: Jul. 5, 2017

Related U.S. Application Data
(63) Continuation of application No. PCT/US2016/012243, filed on Jan. 5, 2016.

(51) Int. Cl. A61K 38/18; A61K 45/06; A61K 9/00; A61L 27/54; A61L 27/60

(52) U.S. Cl. CPC A61K 38/1825; A61L 27/54; A61L 27/60; A61K 9/0014; A61K 45/06; A61L 2300/252

(57) ABSTRACT

Methods for detecting, imaging, analyzing, diagnosing and/or treating cutaneous conditions and dermatoses such as disorders of the skin, subcutaneous tissues, mucous membranes, poorly vascularized tissues and/or other tissue disorders, including erosions, fissures, transient and/or chronic sores, burns, wounds, ulcers, lesions and infections. In particular embodiments, treatments include methods for improving skin and related tissue healing and repair, offloading of damaged tissues and/or increasing angiogenesis in response to specifically diagnosed conditions.

### CROSS REFERENCE TO RELATED APPLICATIONS

This application is a continuation application of PCT Patent Application Serial No. PCT/US16/12243 entitled “THERAPEUTIC ANGIOGENESIS FOR WOUND HEALING,” filed Jan. 5, 2016, which in turn claims priority from the following U.S. Provisional Patent Applications: (1) 62/100,250 entitled “Angiogenic Treatment of Venous Ulcers,” filed Jan. 6, 2015; (2) 62/100,255 entitled “Angiogenic Treatment of Diabetic Foot Ulcers,” filed Jan. 6, 2015; (3) 62/100,259 entitled “Angiogenic Treatment of Vascular Compromised Tissues,” filed Jan. 6, 2015, (4) 62/116,757 entitled “Future of Vascular Medicine,” filed Feb. 16, 2015, and (5) 62/159,841 entitled “Therapeutic Angiogenesis for Wound Healing,” filed May 11, 2015. The disclosures of each of these documents is incorporated by reference herein in their entireties.

### FIELD OF THE INVENTION

The various embodiments herein pertain to the field of detecting, imaging, analyzing, diagnosing and/or treating cutaneous conditions and dermatoses such as disorders of the skin, subcutaneous tissues, mucous membranes and/or other tissue disorders, including erosions, fissures, transient and/or chronic sores, wounds, ulcers, lesions and infections. In particular embodiments, treatments include methods for improving skin and related tissue healing and repair, offloading of damaged tissues and/or increasing angiogenesis in response to specifically diagnosed conditions.

### BACKGROUND OF THE INVENTION

### Description of the Related Art

A cutaneous condition is a medical condition that affects the integumentary system, which is the organ system that encloses the body and includes skin, hair, nails, and related muscle and glands. The skin of an adult weighs an average of between 4 to 5 kilograms (8.8 to 11 pounds), covers an area of approximately 22 square feet, and includes three distinct layers: the epidermis, dermis, and subcutaneous tissue. There are two main types of human skin: (1) glabrous skin, which is the non-hairy skin on the palms and soles (i.e., palmoplantar surfaces), and (2) hair-bearing skin, which incorporates hairs in structures called pilosebaceous units, each with hair follicles, sebaceous glands, and associated arrector pili muscles.

The epidermis 110 is the most superficial layer of skin, and is a squamous epithelium with several strata: the stratum corneum, stratum lucidum, stratum granulosum, stratum spinosum, and stratum basale. Nourishment to the various layers is provided via diffusion from the dermis, as the epidermis is without a direct blood supply. The epidermis contains four cell types: keratinocytes, melanocytes, Langerhans cells, and Merkel cells. Keratinocytes are the major component of the epidermis, constituting roughly 95 percent of the cells therein. The stratified squamous epithelium is maintained by cell division within the stratum basale, in which differentiating cells slowly displace outwards through the stratum spinosum to the stratum corneum, where cells are continually shed from the surface. The stratum basale is a single layer of cells, closest to the dermis. It is usually only in this layer that cells divide. Some of the dividing cells move up to the next layer.

The prickle cell layer (stratum spinosum) is the next layer (8-10 layers of cells). The cells in these layers have lots of desmosomes, which anchor the cells to each other, and contain thick tufts of intermediate filaments (keratin). When the cell shrinks slightly, such as during fixation, the desmosomes from neighboring cells remain tightly bound to each other, and these connections look like ‘prickles’ or ‘spines’, hence the name prickle cells.

The granule cell layer (stratum granulosum) is the next layer (3-5 layers of cells). As the cells move up into this layer, they start to lose their nuclei and cytoplasmic organelles, and turn into the keratinised squames of the next layer. The granules contain a lipid rich secretion, which acts as a water sealant.

In thick skin, a fifth layer (stratum lucidum) is sometimes identified—between the stratum granulosum and stratum corneum layer. It is a thin transparent layer, difficult to recognize in routine histological sections.

The keratinised squames layer (stratum corneum) is the final layer. These are layers of dead cells, reduced to flattened scales, or squames, filled with densely packed keratin. In histological sections these cells are flat and hard to see. The squames on the surface of this layer flake off on a regular basis (making up the main content of household dust).

In normal skin, the rate of production generally equals the rate of loss—i.e., it normally takes about two weeks for a cell to migrate from the basal cell layer to the top of the granular cell layer, and an additional two weeks to cross the stratum corneum. This continuous replacement of cells in the epidermal layer of skin is important. The epidermal layer of the skin and the digestive tract are the two tissues that are directly exposed to the outside world, and therefore are most vulnerable to its damaging effects. In both, there is constant proliferation of cells in the bottom layer (stratum basale) which constantly move up to the top where they are lost. This means damaged cells are continually shed and replaced with new cells.

The dermis is the layer of skin between the epidermis and subcutaneous tissue, and includes two sections, the papillary dermis and the reticular dermis. The superficial papillary dermis interdigitates with the overlying rete ridges of the epidermis, between which the two layers interact through the basement membrane zone. Structural components of the dermis includes collagen, elastic fibers, and extrafibrillar matrix (otherwise referred to as “ground substance”). Within these components are the pilosebaceous units, arrector pili muscles, and the eccrine and apocrine glands. The dermis normally contains two vascular networks that run parallel to the skin surface (i.e., one superficial and one deep plexus), which are connected by vertical communicating vessels. The function of blood vessels within the dermis is at a minimum fourfold: to supply nutrition, to regulate temperature, to modulate inflammation, and to participate in wound healing.

The subcutaneous tissue or “hypodermis” is a layer of fat between the dermis and underlying fascia, and this tissue can be further divided into two components, the actual fatty layer (i.e., panniculus adiposus) and a deeper vestigial layer of muscle (i.e., panniculus carnosus). The main cellular component of this tissue is the adipocyte, or fat cell. The structure of this tissue is composed of septal (i.e. linear strands) and lobular compartments, which differ in microscopic appearance. Functionally, the subcutaneous fat insulates the body, absorbs trauma, and serves as a reserve energy source.

One particular class of cutaneous conditions that affects a substantial portion of the general population are skin ulcers. An ulcer is a sore on the skin or mucous membrane of a patient, generally accompanied by the disintegration of tissues. Ulcers can result in the complete loss of the epidermis, and often portions of the dermis and even subcutaneous fat. Ulcers are most common on the skin of the lower extremities and in the gastrointestinal tract. An ulcer that appears on the skin is often visible as an inflamed tissue with an area of reddened skin.

Ischemic skin ulcers and other wound types can occur when there is poor blood flow in and/or adjacent to a region of skin. Poor blood flow can cause various skin cells to die and damage other tissues. Ulcers can also be caused by exposure to heat or cold and/or irritation, which can cause a sore to form. Ulcers can also be caused due to a lack of mobility, which can cause prolonged pressure on the tissues. This stress in the blood circulation is transformed to a skin ulcer, commonly known as bedsores or decubitus ulcers.

Skin ulcers can appear as open craters, often formed in a round shape, with layers of skin that have eroded. The skin around the ulcer may be red, swollen, and tender. Patients may feel pain on the skin around the ulcer, and fluid may ooze from the ulcer. In many cases, ulcers can become infected, which can include the formation of pus. In some cases, ulcers can bleed and patients can experience fever.

Ulcers typically develop in stages. In stage 1 the skin is red with soft underlying tissue. In the second stage the redness of the skin becomes more pronounced, swelling appears, and there may be some blisters and loss of outer skin layers. During the next stage, the skin may become necrotic down through the deep layers of skin, and the fat beneath the skin may become exposed and visible. In stage 4, deeper necrosis usually occurs, the fat underneath the skin is completely exposed, and the muscle may also become exposed. In the last two stages the sore may cause a deeper loss of fat and necrosis of the muscle; in severe cases it can extend down to bone level, destruction of the bone may begin, and there may be sepsis of joints and an ultimate need for amputation of the affected limb.

Ulcers of the lower legs represent a serious challenge for medicine, especially in the case of diabetic patients. Ulcers of the lower legs are formed mainly as a consequence of chronic venous insufficiency and/or in diabetic patients (i.e., diabetic foot/leg ulcers) as a complication of decreased vasculature and/or microvasculature and a peripheral neuropathy that permits increased trauma to pass unnoticed because of decreased sensation (i.e., diabetic angiopathy, macroangiopathy, microangiopathy and/or neuropathy). Healing of the various types of ulcers is often difficult because insufficient or absent circulation blocks transport of oxygen and nutrients to the cells. As a result, undernourished cells die and necrosis of tissue develops. The lack of circulation also blocks the removal of cell debris and further impedes normal healing processes. Without a healthy, intact skin barrier, the surface of the ulcer is open for infections, which add to the treatment problems. Moreover, ulcers are different from other wounds because whereas normal wounds heal spontaneously over a certain period of time, ulcers, once started, tend to increase in size and wound depth instead of healing. The defective circulation associated with ulcers can cause malnutrition and finally necrosis of the tissue. This in turn, causes a progression of the ulceration which often cannot be compensated by the normal processes of skin repair.

Even when ulcers heal, they often heal very slowly, and in many cases seem not to heal at all. In general, ulcers that heal within 12 weeks are classified as acute, and longer-lasting ones as chronic. Chronic ulcers can be painful, and most patients complain of constant pain at night and during the day. Chronic ulcer symptoms usually include increasing pain, friable granulation tissue, foul odors, and wound breakdown instead of healing.

Treatment of ulcers generally revolves around a desire to promote the normal healing process while avoiding infection of the ulcer, as symptoms tend to worsen dramatically once the wound has become infected. A vast selection of topical formulations is directed to treatment of ulcers, which in most cases are combinations of bacteriostatic or bactericidal drugs, vitamins, herbal constituents, absorbing powders, proteolytic enzymes and others. Treatment typically includes various steps to remove any excess discharge, maintain a moist wound environment, control the edema, and ease pain caused by nerve and tissue damage. The wound or ulcer is usually kept clear of dead tissue through surgical debridement and, in some cases, the creation of skin flaps and/or skin grafting may become necessary. In addition, treatments can involve various approaches to enhance and control skin healing by changing the wound's environment (i.e., use of supplemental oxygen, magnetic fields, altering patient stress and/or location, etc.) or the wound's biochemical activity. Each treatment method can significantly affect the progression and rate of healing as well as the type of tissues formed. In the case of lower extremity ulcers, special exercises and/or compression bandages may be recommended to stimulate circulation of blood in the lower legs. In addition, it is often desirous to offload the treated extremity to prevent further tissue damages and/or promote healing of the damaged tissues.

In many cases, an underlying cause of the ulcer, and/or a major factor contributing to its inability to heal in a timely manner, is impaired blood circulation and/or poor blood flow in and/or adjacent to the region of skin containing the ulcer. Although skin ulcers do not seem of great concern at a first glance, they are worrying conditions, especially in people suffering from diabetes, as they are at risk of developing diabetic neuropathy. Moreover, it is likely that a person who has had a skin ulcer will eventually have it again.

### SUMMARY OF THE INVENTION

Various aspects of the present invention include the realization of a need for improved diagnosis and/or treatment of ulcers and other wounds, especially skin ulcers, burns (i.e., due to excessive heat, cold, chemical, radiation, wind and/or otherwise induced) and/or other wounds resulting from and/or experiencing delayed healing due to ischemic conditions. In various embodiments, skin ulcers and/or other types of damaged skin surfaces can be treated by application of a topical compound which includes one or more angiogenic substances, such as FGF-1. The topical composition may comprise FGF-1 in a concentration between 0.1 to 100%, and this composition may comprise a powder, a gel, an ointment, a lotion, a cream, an oily solution, a suspension, or a semi-solid, and may be applied directly to the surface of the wound and/or impregnated or carried by a dressing, bandage and/or other medical treatment applied to the wound. A dosage of the composition may be administered periodically over an interval of multiple days, may be administered once a day or may be administered multiple times a day, or in the case of a bandage or dressing containing a reservoir of treatment material, may comprise an essentially continuous or periodic “re-application” over a period of time. The number of administrations per day may be, for example, 2, 3, 4, 5, 6 or more. That is, the administration can be applied on a periodic basis, which could include application each day over the course of a treatment period. The treatment period may extend over a period of time necessary to heal one or more ulcers, which may include treatment durations of 14, 28, 42, 70, 91, or 140 or more days.

The topical application of an angiogenic substance, such as FGF-1, to the surface of an ulcer and/or the surrounding epidermal skin surface will desirably induce an angiogenic reaction in one or more of the tissue layers underlying and/or adjacent to the diseased portion of the epidermis, which can potentially increase localized blood flow and/or the effective surface area of the vascular network adjacent to the affected area, as well as induce mitosis (i.e., cell division) or other healing responses of dermal fibroblasts, vascular endothelial cells and/or epidermal keratinocytes. Desirably, the FGF-1 compound will enhance closure of the wound surfaces (i.e., from the wound margins and/or subsurface tissues) while concurrently improving the condition of the underlying vascular network supporting the surrounding layers of the skin and underlying anatomical structures.

In various embodiments, such as where skin or other tissue grafts may be anticipated, the topical application of the angiogenic substance (desirably comprising FGF-1) will desirably initiate an angiogenic cascade in one or more of the tissue layers underlying and/or adjacent to the wound, thereby preparing the wound bed and/or surrounding tissue margins for receiving the potential graft material. When the graft material is placed adjacent to and/or in contact with the wound bed during the graft implantation procedure, the wound bed and/or adjacent tissues will desirably be capable of readily providing nutrients (i.e., via diffusion) to keep the skin graft alive, while concurrently allowing blood vessels to begin to grow from the wound bed into the graft. By the time the graft may no longer be able to survive by diffusion of nutrients alone (which can occur as soon as within a few days after graft implantation), the newly formed vascular network will desirably provide supplemental oxygenation and/or nutrition, with the vasculature (and attendant diffusion therefrom and/or thereto) eventually becoming the primary mechanism for providing oxygen and nutrients to the graft. If desired, the graft material may be “loaded” with angiogenic substances in a similar manner, either prior to, concurrent with and/or after implantation in the wound bed.

In various other embodiments, the topical application of an angiogenic substance to the surface/subsurface of a skin wound and/or surrounding healthy tissues has the potential for “slowing down” and/or halting the process of ulceration for a patient, which might potentially include localized and/or systemic effects that may alleviate various symptoms of the underlying diseases in a systemic manner—including the effects of chronic venous insufficiency and/or diabetes—by reducing, preventing and/or reversing further deterioration of circulation inside the lower legs. Even when a progression of damage may only be slowed and/or temporarily affected by the treatment, such treatment has the potential for slowing the irreversible degradation of the blood vessels, with attendant effects on the healing process.

Some embodiments can include the various treatments described herein in combination with various prosthesis designs to desirably “offload” and/or protect the damaged skin during some or all of the course of treatment. In various embodiments involving lower extremity skin ulcers, special footwear can be utilized that desirably protects and/or offloads the damaged tissue while concurrently applying a therapeutic compound to the surface of the damaged tissue.

In various additional embodiments, methods of assessing and treating damage, wounds and/or ulcers to the skin can include the steps of imaging and/or assessing the damaged tissue and related underlying anatomical areas, assessing the relevant tissue regions, developing a treatment plan and optionally manufacturing a prosthetic device for protecting and/or treating the damaged tissue region.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention:

FIG. 1 depicts an exemplary chart of FGF receptor specificity for FGF-1 through FGF-9;

FIG. 2 is a chart depicting an exemplary Wound Closure Time for a placebo wound and an FGF-1 treated wound;

FIG. 3 depicts representative images of a placebo wound and an FGF-1 treated wound, taken on various days;

FIG. 4 is a pair of charts depicting healing distance versus time for wounds treated with FGF-1 and those treated with a corresponding placebo vehicle;

FIG. 5 is a chart depicting the ulcer healing rate of an FGF-1 treated patient group and a placebo group;

FIGS. 6 and 7 depict images of a pair of equivalent skin ulcers of an FGF-1 treated patient and a placebo patient over a period of time;

FIG. 8 depicts a cross-sectional view of epidermal tissues;

FIG. 9 is a cross-sectional view of skin and subdermal tissues showing the various shallow and deep blood supply and drainage structures;

FIGS. 10A through 10D depict representations of the phases of an exemplary pressure cascade leading to the formation of a compressure sore or ulcer;

FIGS. 11A and 11B depict side and bottom plan views of a foot and foot ulcer;

FIG. 12 depicts a prosthesis model designed to incorporate a depression in the prosthesis proximate a skin ulcer;

FIGS. 13A through 13G depict various views of a foot prosthesis created in accordance with the prosthesis model of FIG. 12;

FIGS. 14A through 14C depict various views of one embodiment of an insert or pad that can serve as a “reservoir” of a angiogenic compound;

FIG. 14D depicts a storage device or “peel pouch” for containing the insert of FIGS. 14A through 14C;

FIGS. 15A and 15B depict an indicator or “tell-tale” incorporated into the insert of FIGS. 14A through 14C;

FIGS. 16A and 16B depict an alternative embodiment of the indicator and insert of FIGS. 14A through 14C;

FIGS. 17A through 17C depict various views of an alternative embodiment of an insert or pad, incorporating a non-permeable and/or inflexible support structure;

FIGS. 18A through 18C depict exemplary steps of placing the insert of FIGS. 17A through 17C within a load-bearing foot prosthesis;

FIG. 19A depicts an alternative embodiment of a prosthesis for use in treating skin ulcers and other wounds with angiogenic medicaments;

FIG. 19B depicts the compression-type prosthesis of FIG. 19A positioned about a patient's lower extremity;

FIG. 19C depicts the compression-type prosthesis of FIG. 19A positioned about a patient's upper extremity; and

FIG. 20 depicts a lateral aspect of a tympanic membrane.

### DETAILED DESCRIPTION OF THE INVENTION

The following description is presented to enable any person skilled in the art to make and use the invention. Various modifications to the embodiments described will be readily apparent to those skilled in the art, and the generic principles defined herein can be applied to other embodiments and applications without departing from the spirit and scope of the present invention as defined by the appended claims. Thus, the present invention is not intended to be limited to the embodiments shown, but is to be accorded the widest scope consistent with the principles and features disclose herein. To the extent necessary to achieve a complete understanding of the invention disclosed, the specification and drawings of all issued patents, patent publications, and patent applications cited in this application are incorporated herein by reference. Although some embodiments are described below, these are merely representative and one of skill in the art will be able to extrapolate numerous other applications and derivations that are still within the scope of the invention disclosed.

It has been determined that FGF-1 and related angiogenic factors possess a remarkable ability to promote and heal damage to the integumentary system, which is the organ system that encloses the body and includes skin, hair, nails, and related muscle and glands. Because much of the integumentary system relies upon diffusive transport of oxygen and nutrition (and also for waste removal) from the vascular system in the body, even minor degradation of the vascular system in the localized region supporting such diffusive transport can severely reduce the integumentary system's ability to protect the body from various kinds of damage, such as acting as a barrier to the external environment, protecting against loss of water, cushioning and protecting deeper tissues, excreting wastes, and/or regulating temperature. Where a significant interruption to the underlying vascular system occurs, the consequences for the overlying integumentary system (and concurrently the overall health of the organism) can be catastrophic, as a damaged or degraded integumentary system poses a significant risk to the organism of disease, infection and ultimately death.

Because the skin is typically an avascular structure, much of the anatomy of the integumentary system relies upon diffusion for nutrition, oxygen and waste removal. The nutrients required to maintain cellular function and viability are supplied to the skin surface by capillary vessels and microvasculature in the subsurface tissue layers proximate to the surface layers. In addition, waste products can be removed via similar mechanisms.

Specifically, while the deeper dermal layers of skin contain heavily vascularized channels, the shallower and/or surface layers of the epidermis rely mainly upon diffusive flow to transport oxygen and nutrients from the blood to the cells of these layers, as well as the transport of various waste products from the cells back to the blood for removal and/or reuse by various other organs. The oxygen, glucose and other nutrients are “dropped off” from the capillaries, and then the nutrients “diffuse” (or otherwise move through the adjacent tissues without being transported in blood vessels) to the adjacent skin cells.

Once glucose and oxygen leave the capillaries, passive diffusion becomes the mechanism of nutrient transport through the intervening anatomical layers. A large concentration gradient may be required for optimal diffusion. The concentration gradient is determined by the utilization of the nutrients by the surrounding tissue population and the concentration of nutrients delivered to the localized anatomical region by the microcirculation. Thus, any decrease in the population of the microvasculature has the potential to create metabolic derangement within the skin layers, leading to degeneration.

Once the nutrients reach the cell, they are taken up and utilized for the manufacture of materials that make up the skin layers. If the cells do not receive enough oxygen, the manufacturing process typically stops and/or significantly reduces. As the nutrient supply is cut off, the cells may begin to die, and the thickness and integrity of the skin tissue can begin to breakdown, which may predispose the skin to degeneration and/or damage.

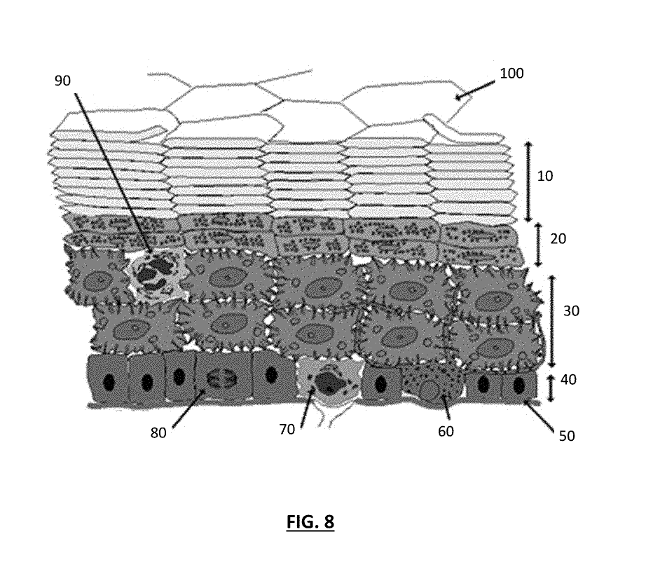

Transport from the vasculature to a cell in the tissue is a two-step process. First, materials flow near to their destination via blood vessels. Then they cover the remaining distance from the blood vessels to the cells primarily via diffusion. The time required for diffusion over large distances is often much longer than that needed for perfusive flow, because diffusion times grow as the square of distance whereas flow times are merely proportional to distance. Under normal conditions, blood is distributed to the capillary bed through an orderly tree-like system of conduits. From there, normal diffusion distances are highly regulated, often to distances less than 50 or 100 μm, and it is generally accepted that the distance that oxygen and other nutrients can diffuse into a given tissue before being metabolized by surrounding cells establishes a maximum distance for “healthy” cells to exist (i.e., “unstressed” cells receiving a desired level of nutrients and oxygen). For example, in the shallower layers of the integumentary system, the epidermal cells with the highest metabolic demand are found closest to the basal lamina, where the diffusion distance is typically shortest, while the surface or “superficial cells” which are more remotely located from the vasculature typically are less active and/or are generally inert or dead (see FIG. 8). In this drawing are included keratinized squames 10, a granule cell layer 20, prickle cell layers 30, a basal cell layer 40, a basil lamina 50, a melanocyte 60, a Merkel cell 70, a dividing cell 80, a Langerhan's cell 90 and a squame about to flake off of the skin surface 100.

In the papillary dermis, the lymphatic system is a closed system (see FIG. 9). Consequently, the lymph circulates outside of the lymphatic system and directly “bathes” the dermic elements—this is the “plasmatic circulation” which constitutes an internal means allowing nutritional exchange to take place. The plasmatic circulation which regulates the lymphatic circulation is under the influence of the blood circulation—its exudation is regulated by blood pressure and by the osmotic pressure of fluids, by nervous influences, endocrine influences, cellular metabolism, by the state of constriction and dilation of the vessels, and finally by the release of H vasodilatory substances emitted in large amounts by irritated cell tissue.

Glucose and oxygen are extremely important to the function and viability of the skin cells. Regardless of the complex interactions taking place in the various skin layers, however, the fact remains that the supply of nutrients, the removal of waste and the overall health of the integumentary system require an intact vascular supply and microvascular capillary network.

Skin is the largest and the most frequently traumatized organ system in the body. Skin injuries are one of the chief causes of death in North America for people between the ages of 1 and 44. In much of the population, the normally healthy vasculature within the dermal layers underlying the integumentary system may be compromised to some degree for a variety of reasons (which can include simple age-related degradation of the patient's body), but for many individuals the level of compromise is of little or no clinical consequence. However, for other individuals, the level of vascular compromise (i.e., systemic or localized) can significantly affect the health and well-being of the patient.

For example, over 5% of individuals over the age of 50 suffer from a vascular deficiency condition known as Peripheral Artery Disease (PAD), in which one or more arteries of the extremities becomes clogged with plaque. PAD most commonly occurs when extra cholesterol and/or other fats circulating in the blood collect in the walls of the arteries that supply blood to the limbs. This buildup, often called plaque, narrows the arteries, often reducing or blocking the flow of blood, which can occur in a localized region, can affect an entire extremity, or in extreme cases can result in systemic consequences. In fact, the number of individuals suffering from PAS is likely a much higher percentage than 5%—while a diagnosis of PAD generally identifies that the degenerative vascular condition has reached a significantly advanced condition, many patients not yet fully diagnosed with PAD will already be suffering from concomitant occlusions and/or blockages in the vasculature and/or microvasculature of one or more extremities as “part and parcel” of the normal disease progression.

Lower Extremity Arterial Disease (LEAD) is a subclass of PAD that is clinically identified by intermittent claudication and/or absence of peripheral pulses in the lower legs and feet. These clinical manifestations reflect decreased arterial perfusion of the extremity. The incidence and prevalence of LEAD increase with age in both diabetic and non-diabetic subjects and, in those with diabetes, increase with duration of diabetes. A common complaint of patients suffering from LEAD, and especially true of diabetic patients, is the patient's proneness to infection, ulcerations and poor healing of skin sores and ulcers. Moreover, LEAD in diabetes is compounded by the presence of peripheral neuropathy and insensitivity of the feet and lower extremities to pain and trauma. The combination of impaired circulation and impaired sensation in such patients can easily lead to ulceration and infection, often progressing to osteomyelitis and gangrene which may necessitate amputation of part or all of the affected extremity.

In the case of diabetes, the disease burden of the diabetic foot that develops an ulcer is substantial. From the estimated 24 million Americans who have diabetes, the annual prevalence of foot ulcers in this population ranges from 4-10%, or approximately 1 million to 2.5 million subjects suffering with foot ulcers each year. Complications from non-healing ulcers, including infection and gangrene, are the leading causes of hospitalization in patients with diabetes mellitus. The most costly and feared consequence of a foot ulcer is amputation of the limb. Each year, an estimated 82,000 limb amputations are performed on diabetic patients in the U.S.

The effects of LEAD and diabetes together account for approximately 50% of all non-traumatic amputations in the United States, and it is acknowledged that a secondary amputation within several years after the first is exceedingly common. Moreover, mortality is increased in patients with LEAD, particularly if foot ulcerations, infection, or gangrene occur, and three-year survival after an amputation is <50%. Prevention is an important component of LEAD management, because by the time LEAD becomes clinically manifest, it may be too late to salvage an extremity, or it may require more costly resources to improve the circulatory health of the extremity. While surgically invasive revascularization procedures of the larger arteries can improved perfusion and flow to the lower extremity, such procedures are often not recommended in a large proportion of patients, and even where successful have not had an appreciable reduction in the frequency of amputation experienced by revascularized patients.

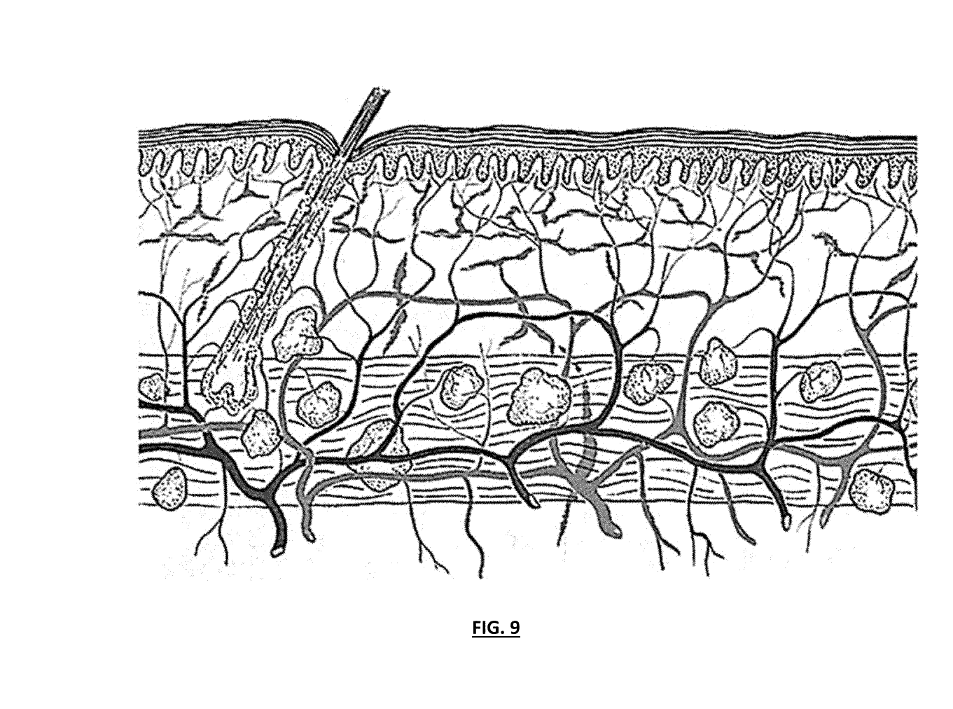

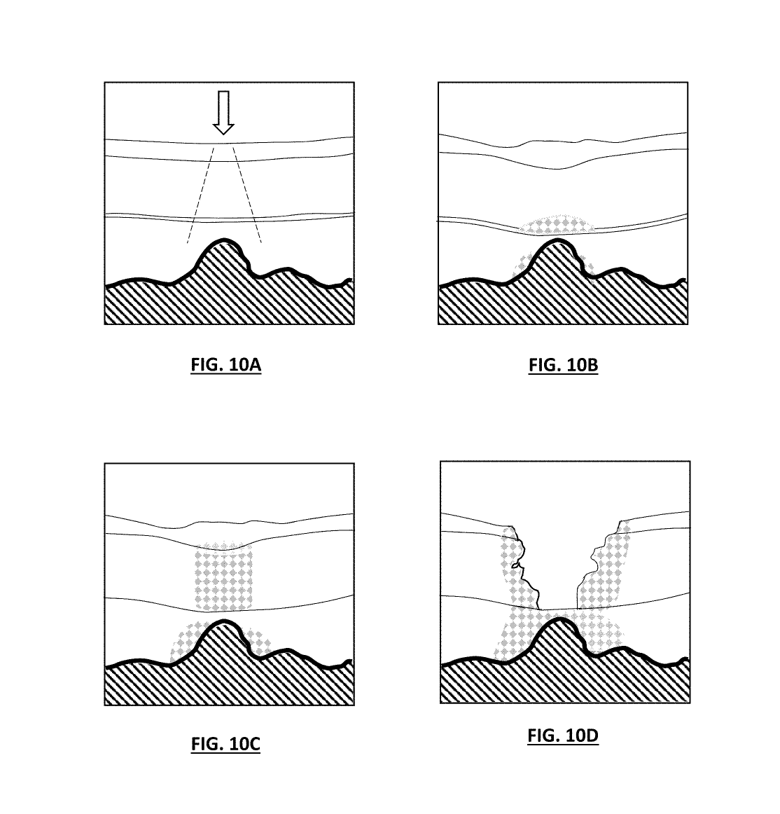

Another common disorder of the integumentary system are pressure sores or ulcers, commonly referred to as “bedsores.” One of the most prevalent skin injuries affecting a large percentage of the patient population, bedsores are injuries to the skin and underlying tissue resulting from prolonged pressure on the skin, which are caused by pressure against the skin that limits blood flow to the skin and nearby tissues (FIG. 10A). In these cases, a localized area of tissue necrosis develops (FIG. 10B) when the soft tissue is compressed for a prolonged period (often between a bony prominence and an external surface), forming the bedsore. Bedsores can range from superficial inflammation that extends into the dermis (FIG. 10C) to an extensive ulcer occasionally involving underlying bone (FIG. 10D).

Bedsores are especially prevalent in areas of the body that aren't well-padded with muscle or fat and that lie over a bone, such as the spine, tailbone, shoulder blades, hips, heels and elbows. When the skin and underlying tissues are trapped between the underlying bone and a surface that presses on the skin (such as a wheelchair or a bed surface), this pressure may be greater than the pressure of the blood flowing in the capillaries and/or other vessels that deliver oxygen and other nutrients to the tissues, potentially impeding and/or halting the flow of such materials. When the pressure is sustained for a sufficient period of time, which can be as little as a few hours, the skin and underlying structures can become damaged and/or eventually die. Other factors contributing to the severity of bedsores can include friction damage, if the skin is being dragged across a surface during movement, and shear damage (i.e., compression, tension and/or shear forces) applied to the skin and underlying tissues—motion that may injure tissue and blood vessels, making the site more vulnerable to damage from sustained pressure.

Bedsores and other types of pressure ulcers are one of the most debilitating and costly problems associated with hospitalization, including surgical procedures involving long term care and rehabilitation, immobilization and/or disabling conditions such as spinal cord injury (SCI). Pressure ulcers can interfere with every aspect of a physically disabled individual's life, from active participation in the rehabilitation program to returning to an active role in the community. Pressure ulcers are found in 20-30% of individuals with SCI, 43% among nursing home residents, and 15% of persons with acute injuries. In 2006, it was estimated that persons with SCI who have pressure ulcers incur hospital charges three to four times those of other individuals with SCI, and averaged at least an additional $48,000 in health care costs. In 2010 it was calculated that, for the most severe sores (i.e., grade 4), the average hospital treatment cost was more than $129,000 for hospital-acquired ulcers during one admission, and $124,000 for community-acquired ulcers over an average of 4 admissions sores (i.e., grade 4). Since hospital charges relate directly to the number of days in treatment, reducing the length of a hospital stay through more effective treatment of pressure ulcers, skin ulcers or other skin wounds could mean a significant savings for the patient, the health care delivery system, and the third party payer. Moreover, a nonsurgical treatment that promotes healing in a shorter time would reduce the hospital stay, recovery time, costs, and complications associated with surgical skin grafting. In addition, malpractice suits associated with the development of pressure ulcers average $250,000 per settlement, reportedly totaling at least $65,000,000 annual in the U.S. alone.

People most at risk of pressure ulcers and/or bedsores are those with a medical condition that limits their ability to change positions, requires them to use a wheelchair or confines them to a bed for a long time. Bedsores can develop quickly and are often difficult to treat. Bedsores fall into one of four stages based on their severity, which the National Pressure Ulcer Advisory Panel (a professional organization that promotes the prevention and treatment of pressure ulcers) defines each stage as follows:

STAGE I:
The skin is not broken.
The skin appears red on people with lighter skin color, and the skin doesn't briefly lighten (blanch) when touched.
On people with darker skin, the skin may show discoloration, and it doesn't blanch when touched.
The site may be tender, painful, firm, soft, warm or cool compared with the surrounding skin.

STAGE II:
The outer layer of skin (epidermis) and part of the underlying layer of skin (dermis) is damaged or lost.
The wound may be shallow and pinkish or red.
The wound may look like a fluid-filled blister or a ruptured blister

STAGE III: the ulcer is a deep wound
The loss of skin usually exposes some fat.
The ulcer looks crater-like.
The bottom of the wound may have some yellowish dead tissue.
The damage may extend beyond the primary wound below layers of healthy skin.

STAGE IV: the ulcer shows large-scale loss of tissue
The wound may expose muscle, bone or tendons.
The bottom of the wound likely contains dead tissue that's yellowish or dark and crusty.
The damage often extends beyond the primary wound below layers of healthy skin

Regardless of the cause(s) of damage or wounds to the integumentary system, an important feature of a healthy integumentary system is the ability of the body to heal such damage or wounds. In normal, healthy patients, the epidermal skin layers typically exist in a “steady-state” equilibrium—forming a protective barrier against the external environment. An injury to the skin sets into motion a set of complex biochemical events in a closely orchestrated cascade, which seeks to repair the damage. The response to injury is an essential innate host immune response for restoration of tissue integrity. Tissue disruption in higher vertebrates desirably results in a rapid repair process leading to a fibrotic scar. Wound healing, whether initiated by trauma, microbes or foreign materials, proceeds via an overlapping pattern of events including (1) hemostasis and coagulation, (2) inflammation, (3) proliferation (including epithelialization and formation of granulation tissue), and (4) matrix and tissue remodeling. The process of repair is mediated in large part by interacting molecular signals, primarily cytokines, which motivate and orchestrate the manifold cellular activities which underscore inflammation and healing.

The specific cellular activities and interrelationships in the wound healing cascade are extremely complex, but as a relatively simplified explanation, the following steps occur. Within the first few minutes after a skin injury, platelets adhere to the site of injury, become activated, and aggregate (i.e., they join together); followed by activation of the coagulation cascade which forms a clot of aggregated platelets in a mesh of cross-linked fibrin protein. This clot stops active bleeding (i.e., “hemostasis”).

The initial injury also triggers an acute local inflammatory response followed by mesenchymal cell recruitment, proliferation and matrix synthesis. During the “inflammation” phase, bacteria and cell debris are phagocytosed and removed from the wound by white blood cells. Platelet-derived growth factors (stored in the alpha granules of the platelets) are released into the wound that cause the migration and division of cells during the proliferative phase. Failure to resolve such inflammation can lead to chronic non-healing wounds, whereas uncontrolled matrix accumulation, often involving aberrant cytokine pathways, can lead to excess scarring and fibrotic sequelae

Clearance of debris, foreign agents and any infectious organisms promotes resolution of inflammation, apoptosis, and the ensuing repair response that encompasses overlapping events involved in granulation tissue, angiogenesis, and re-epithelialization. Within hours, epithelial cells begin to proliferate, migrate and cover the exposed area to restore the functional integrity of the tissue. Re-epithelialization is seen as critical to optimal wound healing, not only because of reformation of a cutaneous barrier, but also because of its role in wound contraction. This “proliferation” phase is characterized by angiogenesis, collagen deposition, granulation tissue formation, epithelialization, and wound contraction. In angiogenesis, vascular endothelial cells desirably form new blood vessels. In fibroplasia and granulation tissue formation, fibroblasts grow and form a new, provisional extracellular matrix (ECM) by excreting collagen and fibronectin. Concurrently, re-epithelialization of the epidermis occurs, in which epithelial cells proliferate and ‘crawl’ atop the wound bed, providing cover for the new tissue. Immature keratinocytes produce matrix metalloproteases (MMPs) and plasmin to dissociate from the basement membrane and facilitate their migration across the open wound bed in response to chemoattractants. The migration of epithelial cells occurs independently of proliferation, and depends upon a number of processes, including growth factors, loss of contact with adjacent cells, and guidance by active contact.

During wound contraction, myofibroblasts decrease the size of the wound by gripping the wound edges and contracting using a mechanism that resembles that in smooth muscle cells. When the cells' roles are close to complete, unneeded cells undergo apoptosis. During maturation and “remodeling,” collagen is remodeled and realigned along tension lines, and cells that are no longer needed are removed by apoptosis.

While the process of wound healing in the skin of a healthy individual is a relatively straightforward process, the same cannot be said for wound healing in the skin of an individual suffering the effects of vascular compromise. The complex skin healing process in such compromised individuals is often very fragile, and is susceptible to interruption or failure at many points—leading to the formation of non-healing chronic wounds. In fact, there are a wide variety of factors that can interfere with skin healing and the formation of such non-healing chronic wounds, including diabetes, venous or arterial disease, infection, and metabolic deficiencies of old age. Faulty or impaired healing has been repeatedly labelled the most prominent factor in these lesions, and thus speeding up the rate of regenerative healing would be expected to reduce both the likelihood and effect of other secondary complications.

Of all the potential complications affecting the ability of the skin to heal, the condition of the underlying vascular support network is arguably one of the most important. Virtually every step in the wound healing process either relies upon and/or is directly influenced by the conditions of the underlying vasculature. In many cases, an underlying vascular abnormality and/or insufficiency can significantly reduce and/or eliminate the body's ability to heal a skin wound. For example, the vasculature is the cells' primary source of oxygen and nutrition, as well as a primary channel for of waste removal. A lack of nutrition can inhibit or prevent normal repair and/or replacement of cellular structures, while insufficient oxygen can result in cell death. In a similar manner, a lack of sufficient waste removal can result in a buildup of wastes within and/or between the cells—potentially degrading and/or inhibiting the cells' ability to function and properly repair damage. Moreover, the vasculature is the primary transport pathway for numerous cells and materials necessary for protection of the organism and repair of the skin wound—so an interruption in the vascular transport mechanism means an interruption in the availability of these cells/materials as well.

Another factor that can significantly affect the ability of skin wounds to heal is the presence or absence of infection. Where vascular compromise is a concern, skin wounds can be predisposed to infection because of the underlying vasculopathy as well as a related immunopathy (i.e., diminished neutrophil function). Once an infection has become established in a skin wound, the underlying vascular and/or microvascular compromise can further complicate treatment, as phagocytic cells will have limited access to the region and systemic antibiotics will generally have a poor concentration in the infected tissues. Moreover, infected skin wounds heal much more slowly than their non-infected counterparts.

Currently, the most effective conservative methods of treating skin wounds, including small or large ulcers, involve removing pressure from the affected area, providing a dressing or other covering over the wound to collect wound exudate and protect and hydrate the wound area, and allowing the body to naturally heal the skin. However, it would be desirous to incorporate additional interventions that could facilitate these processes and possibly even speed up regenerative skin healing, so as to reduce the costs, length of medical treatment and morbidity commonly associated with pressure ulcers. An optimal intervention in many cases would desirably include a medical device or prosthesis capable of offloading a skin ulcer or pressure sore to a sufficient degree to facilitate healing, in combination with an intervention that enhances and optimizes the ability of the skin to regenerate at a rate equal to or greater than normal reparative healing. Additional advantages could include a medical device capable of offloading a pressure sore while allowing the patient to ambulate with a lesser or greater degree of freedom, especially where the medical device can facilitate periodic application of an angiogenic compound to the wound.

In various additional embodiments, an antibiotic, antiseptic, analgesic substance and/or other medicament could be incorporated into the angiogenic compound for topical application to the skin wound. A wide variety of antibiotics, treating agents and/or other infection fighting agents are available for topical application, which can include antibiotics suitable for treatment of infections of gram negative and/or gram positive bacteria. If desired, a plurality of antibiotic and/or infection fighting agent types can be incorporated, including betadine, peroxide-based preparations, ethacridine lactate, mupirocin (Bactroban), cadexomer iodine, providone iodine, honet-based preparations, silver-based p[reparations, enzymatic cleansers, chloramphenicol-containing ointments, framycetin sulphate ointment and/or herbal ointments. In various embodiments, a pharmaceutically effective amount of pexiganan cream (commonly known as Locilex 0.8% cream, which is commercially available from Dipexium Pharmaceuticals, Inc., of New York, N.Y.) can be combined with angiogenic factors such as FGF-1 and topically applied to a surface of the skin ulcer and/or the surrounding healthy tissues. This cream has the ability to kill microbial targets through disruption of the bacterial cell membrane permeability, which is effective against a broad spectrum of gram-positive, gram-negative, aerobic, and anerobic bacteria, as well as fungi, and pexiganan has particular utility against methicillin-resistant staphylococcus aureus (or MRSA), vancomycin-resistant enterococcus (or VRE), extended-spectrum beta-lactamases (or ESBL) and multi-drug resistant (or MDR) bacteria.

Current approaches toward healing for many types of skin wounds can range from environmental control through dressing applications to surgery in the form of skin grafting and skin flaps. The healing process depends on the size of the ulcer and patient compliance. Clinically, both deep (full-thickness) and shallow (partial thickness) pressure ulcers and other skin wounds are of concern. In most cases, partial thickness wounds (Grade 1 and 2) can be treated with wound dressings, rather than requiring skin grafts, since the lost epithelium in such “minor” wounds is expected to regenerate on its own with little or no dermal contraction. Immediate concerns with shallow pressure ulcers include blood loss, bacterial invasion, and fluid loss in partial thickness wounds. Shallow wounds typically heal naturally, however, many of these skin ulcers can progress to deeper wounds due to pathology or continual irritation. In any case, speeding up the regenerative healing would be beneficial. Therefore, there is a place for regenerative treatments and/or systems even for these shallow wounds.

Full thickness wounds (Grades 3 and 4) generally involve a loss of the epithelium and dermis. These usually necessitate more active treatments than simple wound dressings. The dermis normally does not naturally regenerate itself, and healing occurs primarily through the development of granulation tissue and scar, causing the wound area to contract and lose its elasticity. In various embodiments, one optimal wound dressing could comprise a dressing that provides a scaffolding structure to promote the development of a new dermis over which the epidermis could grow without any contraction.

A wound dressing, when it is used, can enhance healing in a number of ways. Skin healing can be altered by changing the configuration (pore size, porosity, fiber diameter), the surface (composition, charge, surface energy), the biochemical activity (incorporation of biochemical factors), or the degradation or drug delivery rate of a wound dressing. The goal in virtually all cases is tissue regeneration and at the fastest possible rate. In various embodiments, a wound dressing (if there is one) might desirably be degradable. Dressing change regimens for deep skin ulcers can take from six weeks to six months of bed rest to heal. Typically the choice of last resort, surgical interventions can cause additional unwanted damage to the affected tissues, can result in co-morbidities such as infection or damage at a donor tissue site, and typically involve higher costs for surgery and a lengthy post-operative healing period. Moreover, surgical interventions in the form of pedicle flaps and/or skin grafts may not be ideal solutions. For skin ulcers, skin flaps (the usual method of choice) do not always take and there are a limited number of donor sites available for such tissues.

Various embodiments described herein relate to methods for imaging, diagnosing, quantifying, assessing, and/or treating or ameliorating painful and/or degenerative conditions of the skin, including those that ultimately involve ulcers and/or other skin wounds. Embodiments can include classifications of skin cell and related tissue nutrition deficit, pathological conditions and/or associated degeneration and/or chronic conditions that can be based on specific parameters associated with hypoperfusion, hypoxia, and ischemia. Further embodiments relate to treatments for alleviating the state of hypoperfusion, hypoxia, and ischemia in patients in which alleviation of said hypoperfusion may lead to therapeutic improvement.

Various embodiments described herein can be employed to diagnose, assess, quantify and/or treat pathologies that can eventually lead to deficient nutrition to and/or waste removal from tissues such as the skin. In one initial step, anatomical image data could be obtained of an individual patient's anatomy. This image data can be derived from a wide variety of sources, including MRA (magnetic resonance angiography), MRI (magnetic resonance imaging), x-ray imaging, cone beam CT, digital tomosynthesis, and ultrasound, CT scans or PET or SPECT scans, as well as many others known in the art. Once image data is acquired, one or more regions of interest (ROI) of the image data can be identified and analyzed in a variety of ways, and the analyzed results can be compared to a defined value and/or standard and utilized to diagnose, assess and/or quantify a pathology. If desired, the analysis and diagnosis can be used as guidance for treating the patient. In various other embodiments, the results can be compared to values derived or obtained from a reference database of healthy and/or diseased patients. In other alternative embodiments, a relative assessment of such values within an individual patient can be conducted, which may be used to identify abnormal and/or anomalous readings, which may be indicators of relative deficiencies.

Various embodiments described herein can be employed to diagnose, assess, quantify and/or treat pathologies that can eventually lead to deficient nutrition to and/or waste removal from skin layers or other tissues. The nutrient supply to the skin can potentially be blocked at various stages of the route. The feeding arteries or other vascular structures themselves can narrow due to atherosclerosis with resultant ischemia of a localized region or extremity. With less blood flowing through the extremity, less oxygen and nutrients may be available to diffuse into the affected skin layers creating hypoxia, reduced activity, reduced viability and/or cell death. In addition to and/or instead of narrowing of the major vessels, a reduced number and/or size of vessels and/or lower density of the blood flow within the anatomical layers adjacent to the skin can be a primary reason for the loss of nutrients and the onset of a degenerative or chronic skin condition and/or loss of healing ability. Trauma can disrupt blood and/or nutrition flow. Degenerative skin conditions due to nicotine and aging can also demonstrate a loss of nutritive blood vessels in the area supplying nutrients. Eventually, intervening tissue and/or scarification could become a hindrance to the diffusion of nutrients, potentially creating another obstacle to proper skin nutrition.

In one exemplary embodiment, diagnosed dermal hypoperfusion can be treated by increasing perfusion in identified area(s), such as by injection of a composition that includes an angiogenic factor. In preferred embodiments, injection can be directly into healthy tissues proximate to the identified area or areas of hypoperfusion. The identified area or areas can be accessed via a transdermal approach with a surgical access and delivery device such as a surgical access needle extending through the patient's skin and overlying soft tissues in a minimally-invasive manner. The composition could then be introduced into the anatomy through the delivery device.

In another exemplary embodiment, diagnosed dermal hypoperfusion could be treated by increasing perfusion in identified area(s), such as by topical application of a composition that includes an angiogenic factor. In preferred embodiments, the composition could be applied to the surface of the wound or ulcer and/or to the surface of the healthy tissues proximate to the wound or ulcer, as well as to identified area or areas of hypoperfusion.

In various alternative embodiments, hypoxic and/or ischemic skin disease could be treated by increasing perfusion in the affected area, such as by topical application of a composition that includes an angiogenic factor, by injection of a composition that includes an angiogenic factor, and/or by various combinations thereof. In preferred embodiments, topical application and/or localized injection could be proximate to the wound or ulcer. In other embodiments, introduction of angiogenic compounds could be undertaken into and/or adjacent to other anatomical structures, including major arteries and/or veins supplying/removing blood from/to the affected extremity and/or other skin region. In some embodiments, a localized delivery system capable of forming a gel-like structure might preferably be used to deliver the angiogenic factor.

In various preferred embodiments, the delivery system could include components of extracellular matrix that provide conditions suitable for angiogenesis. In some embodiments, said extracellular matrix components may be hyaluronic acid fragments. In other embodiments, said extracellular matrix components may be derivatives of collagen, or perlecan. In various embodiments, the gel-like structure could include a polymer capable of slow release such as a poloxamer block copolymer (Pluronic®, BASF), a basement membrane preparation (Matrigel®, BD Biosciences) or a collagen-based matrix such as described by U.S. Pat. No. 6,346,515, which is incorporated herein by reference.

In another exemplary embodiment, the diagnosis of hypoxic or ischemic skin disease as a disorder could be made by a multi-step test of firstly excluding patients with a set exclusion criteria, and further selecting patients having documented hypoperfusion, hypoxia, or ischemia of the affected areas. Various embodiments described herein include the realization that the health of avascular or partially-vascularized tissues may be dependent, at least in part, upon diffusive nutrient flow from and/or waste product flow towards adjacent vascularized regions. Where such adjacent vascularized regions may experience perfusion insufficiencies, the relevant diffusive flows may be partially or completely disrupted, which may result in tissue degradation of the adjacent avascular and/or partially-vascularized tissues. Desirably, where the perfusive insufficiency of the vascular region can be reversed or ameliorated as described herein, the diffusive nutrient/waste flow can be restored to some degree, which desirably results in slowing, halting and/or reversing of the tissue degradation process.

Embodiments described herein provide hypoxic and/or ischemic skin disease as a defined disease subset, in which patients may be specifically classified that are amenable to treatment with treatments capable of stimulating perfusion, cell regeneration and/or preventing or slowing further vascular degeneration. Specifically, in one embodiment, hypoxic and/or ischemic skin disease is diagnosed as partial or complete stenosis of one or more blood vessels and/or microvascular regions associated with the treatment area.

Embodiments of the invention can also be directed to methods of diagnosing a condition responsible for a degenerative or chronic skin condition, which may include one or more of the following steps:
a) assessing a patient by one or more of the following steps:
   (i) classifying patency of said one or more major vessels;
   (ii) determining blood perfusion in the anatomical areas supplied by said major vessels;
   (iii) determining an extent of localized blood flow proximate to a wound area demonstrating degenerative or chronic characteristics;
b) correlating data collected from a(i) with data collected from a(ii) and with data collected from a(iii));
c) producing an overall index of correlation; and
d) comparing said index of correlation with an index of correlation generated from a healthy population.

In another exemplary embodiment, a method of diagnosing a condition responsible for a degenerative or chronic skin condition could include one or more of the following steps:
a) assessing a patient by one or more of the following steps:
   (i) obtaining image data of one or more anatomical regions including at least one degenerative and/or chronic skin condition;
   (ii) identifying one or more regions of interest within the image data;
   (iii) analyzing the one or more regions of interest to identify one or more areas of dermal hypoperfusion proximate to an area encompassing the degenerative and/or chronic skin condition; and
   (iv) diagnosing the patient with said hypoperfusion proximate to the area of the degenerative and/or chronic skin condition.
      Wound Healing with FGF-1

Human FGF-1 is a 141 amino acid monomeric protein devoid of any requisite post-translational modifications such as glycosylation. It was first isolated in its pure form in the early 1980s in the laboratory of Dr. Ralph Bradshaw at the Washington University School of Medicine in St. Louis. The amino acid sequence of the protein was subsequently determined at Merck by a team led by Dr. Ken Thomas. Dr. Thomas then went on to determine the three dimensional structure of FGF-1. Human FGF-1 can be made as a recombinant protein in E. coli and its ability to bind strongly to heparin allows for a relatively easy purification by heparin affinity chromatography. The heparin binding ability is one reason FGF-1 has a potential to be a potent wound healing agent, as it can stay resident in the wound bed for days bound to heparin moieties found in abundance on basement membranes of damaged tissues. The simplicity of the FGF-1 structure also makes it a very stable molecule and in the presence of heparin—FGF-1 is stable for 18 months at 4° C., a desirable quality for a pharmaceutical.

FGF-1 is a member of a family that includes 22 FGF proteins. FGF-2 or basic FGF has also been extensively characterized and was in development for the treatment of stroke. FGF-7 or keratinocyte growth factor is an FDA approved drug and is used to regenerate the epithelium inside of the mouths of cancer patients undergoing chemotherapy. The 22 members of the FGF family interact with seven distinct FGF cell surface receptors. FGF-1 is the only member of the family of 22 FGFs that binds to all 7 receptor isoforms with high affinity (see FIG. 1). Also, FGF-1 is the only growth factor having a potential to be mitogenic for dermal fibroblasts, vascular endothelial cells, and epidermal keratinocytes, the three major cell types present in skin. These structural properties and biologic activities make it an extremely attractive therapeutic agent to promote dermal healing.

FGF-1 is present in a wide range of tissue types and is implicated in a broad array of biological functions including embryonic development, cell proliferation and differentiation, and tissue repair including dermal wound healing. As mentioned above, FGF-1 is the only growth factor known to be mitogenic and chemotactic for the three major cell types present in skin: dermal fibroblasts, vascular endothelial cells, and epidermal keratinocytes. FGF-1 is also mitogenic for pericytes, capillary smooth muscle cell-like cells that decorate the microvasculature and are a necessary component for the formation of new capillaries. Further, FGF-1 is capable of in vivo stimulation of angiogenesis, granulation tissue formation, and the growth of new epithelium, as measured by quantitative histomorphometric analyses.

In a variety of preliminary assessments, FGF-1 has induced angiogenesis in specially designed assays for blood vessel growth employing embryonic chick chorioallantoic membranes and rabbit corneas, and FGF-1 has demonstrated an ability to accelerate wound healing in laboratory animals, with low dose therapy resulting in a two-fold increase in the rate of full-thickness wound closure. Moreover, topically applied recombinant human FGF-1 has been determined to promote the closure rate of 1.6 cm circular full-thickness excision wounds in genetically diabetic mice.

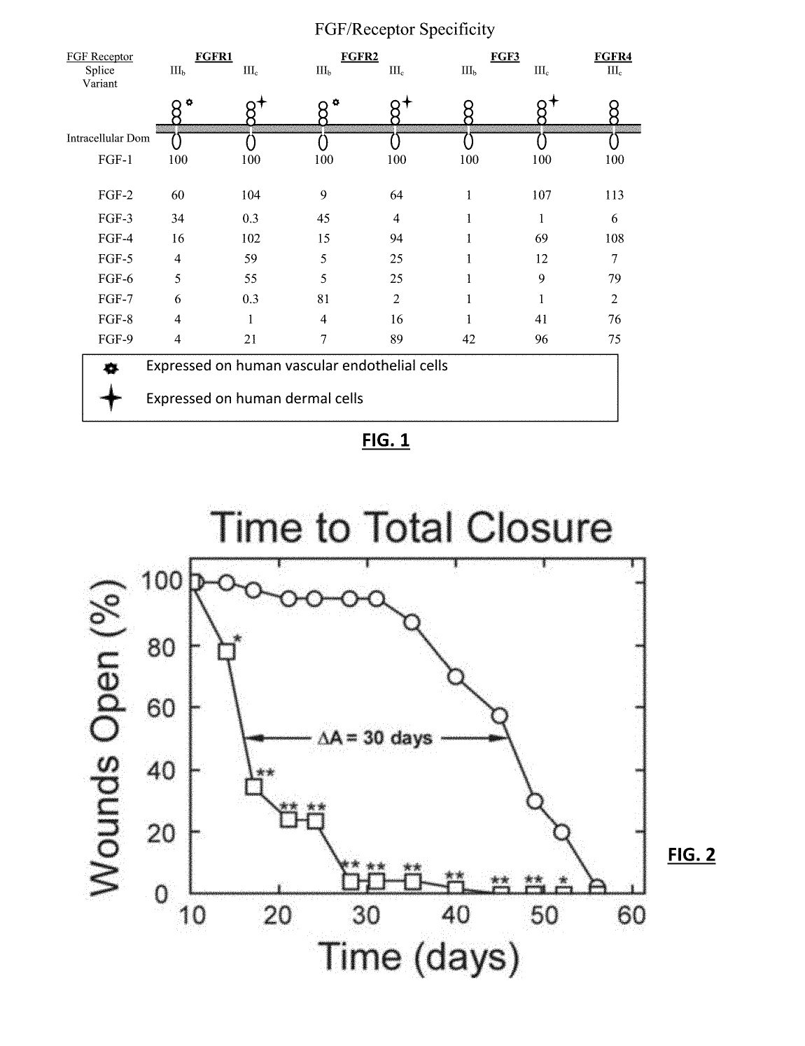

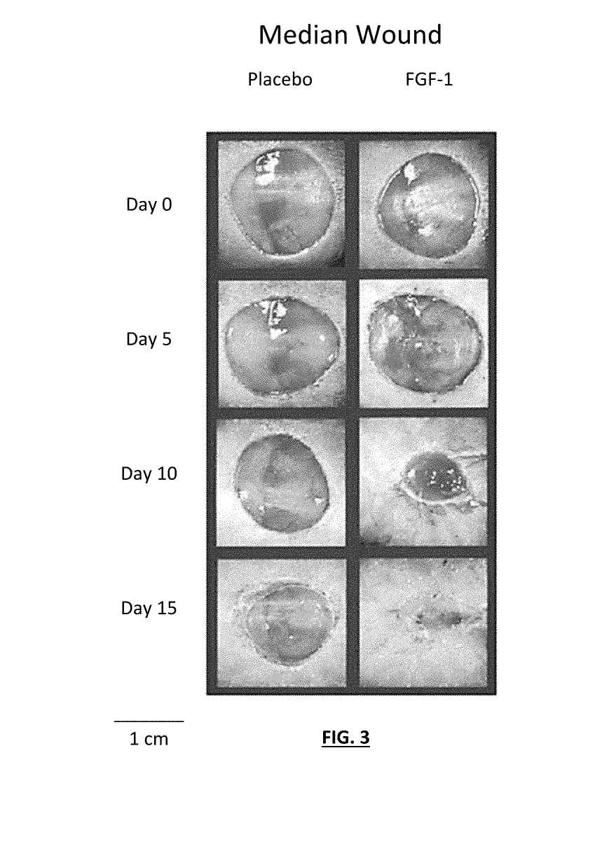

During one exemplary animal study, skin wounds were treated on the day of injury (day 0) and again on days 3 and 7 with 3 μg/cm2 FGF-1 with heparin or a corresponding placebo vehicle, and covered with a bio-occlusive dressing to keep the wound moist. The placebo vehicle also contained an equivalent concentration of heparin as in the active arm. Surface areas were measured twice weekly by image analysis of open wound tracings. FGF-1 dramatically accelerated wound healing in the animal model and culminated in a very significant decrease in time to total closure, which in FIG. 2 is depicted as an average decrease in the time to close of 30 days, with FIG. 3 depicting representative images of a placebo wound and an FGF-1 treated wound, photographed on days 0, 5, 10 and 15. In other animal studies, FGF-1 has demonstrated a measureable improvement in the healing of skin wounds of diabetic animals, with FGF-1 (as compared to placebo) induces faster healing, higher rates of wound closure, increased levels of fibroblasts, vasculature, and collagen deposition, accompanied by increased levels of transforming growth factor-beta (TGF-β) and proliferating cell nuclear antigen (PCNA), a measure of cell proliferation.

In various embodiments, an angiogenic compound, such as FGF-1, can be included as part of a treatment regime for a skin wound, ulcer or other chronic skin condition. Such treatment can include topical application of the angiogenic compound to the surface of the wound, to the margin(s) of the wound and/or to the surface of surrounding healthy and/or undamaged skin or other tissues. Desirably, the angiogenic compound will induce cell growth and/or growth and/or expansion of the various vascular structures/network underlying and/or adjacent to the damaged tissues, providing improved oxygen, nutrients and/or waste removal for at least a portion of the damaged tissues.

In various embodiments, angiogenic effects induced in a patient have the potential of creating one or more of the following: (1) a localized improvement in the vasculature and/or microvasculature of the extremity (or other anatomical locations, including non-extremity areas) proximate to the skin wound, (2) a systemic or localized improvement by artificially inducing the body to create a collateral flow around an occlusion or blockage in the vasculature of the affected limb (i.e., artificially inducing a “natural bypass”), and/or (3) various combinations thereof. For example, the angiogenic effects of FGF-1 might induce the vasculature and/or capillaries to grow more proximate and/or closer to the area of skin damage (i.e., recruiting blood vessels into previously unperfused/underperfused regions or regions where perfusion has become deficit), which desirably reduces the distance that nutrients and/or oxygen must travel via diffusion. In other embodiments, the angiogenic effects might induce the vasculature and/or capillaries to grow more densely in areas proximate and/or closer to the area of skin damage, which could potentially increase the overall availability and/or concentration of nutrients and/or oxygen available for use in repairing the localized area of skin damage. In still other embodiments, the angiogenic effects might induce the vasculature to repair, bypass and/or reroute a damaged and/or degraded area of vasculature and/or microvasculature, thereby potentially improving localized and/or systemic vascular flow within the extremity and/or other anatomical area of the patient's body. In another embodiment, the angiogenic effects might induce the vasculature to open compressed vascular pathways, thereby potentially improving local and/or system vascular flow within the extremity and/or other anatomical area of the patient's body. In another embodiment, the angiogenic effects might induce growth of the vasculature and/or microvasculature towards healthier sources and/or areas of the vasculature (i.e., redirecting flow from well-perfused vessels to poorly perfused vessels and/or regions), so as to route additional nutrients and/or oxygen to the treatment area. In another embodiment, the angiogenic effects might induce growth of additional vascular linkages and/or interconnections between the superficial and deep plexus layers of the dermis and/or other subdermal tissues. In other embodiments, various combinations of the previously disclosed angiogenic effects might occur.

In addition to the various angiogenic effects described herein, in various embodiments the application of FGF-1 to the damaged skin structures will desirably induce growth and/or repair of cells of the various skin layers, including within one or more of the dermal fibroblasts, the vascular endothelial cells and/or the epidermal keratinocytes. For example, application of FGF-1 can markedly increase the proliferation of fibroblasts that give rise to granulation tissue, which tills up a wound space/cavity early in the wound healing process. Moreover, FGF1 can activate and/or signal a cascade of cell proliferation, such as by initiating the biological signals of FGF2 and FGF7, which in turn signal additional healing responses.

### Anatomical Imaging and Structural/Functional Analysis

Depending upon the specific tissue structure(s) concerned, the diagnosis and/or treatment methods and systems described herein may include the selection and analysis of a plurality of relevant tissue structures. For example, where the diagnosis and/or treatment of a skin condition of a patient's extremity is of interest, the methods and systems described herein can include the imaging and analysis of some portion of relevant tissues and/or the entirety of the extremity of interest. Depending upon the physician's preference and/or the relevant clinical situation, diagnosis of hypoperfusion of some portion of the patient's vascular system might indicate a need for further treatment, as described herein.

In various embodiments, the various concepts described herein can optionally include the use of image data obtained of a patient's anatomy, which can include non-invasive and/or limited-invasive (i.e., contrast enhanced and/or minimally-invasive) sources of image data of the patient. The various embodiments and concepts disclosed herein also contemplate the use of technologically improved software and/or imaging hardware and systems that can provide high-quality images without the use of contrast injections and/or other exogenous agents, including those developed in the future. In various embodiments, the efficient detection, analysis and diagnosis of ischemic skin conditions, vascular blockages and/or occlusions, diffusive insufficiencies and/or other tissue-related pathologies will typically be dependent upon the quality and resolution of image data acquired of the patient's anatomy. Where the diagnosis is focused on nutrition to an extremity and/or localized skin region, the relevant patient image data will desirably include anatomical image data of the localized skin region and an area surrounding the region of interest, as well as the extremity, the skin region and any surrounding anatomy, as desired.

A unique challenge posed by various embodiments described herein can relate to unique anatomical features of the particular anatomy of interest. Unlike typical anatomical imaging studies, various regions of interest particularly relevant to the present invention might include image data of vasculature and other anatomical structures located inside and/or outside of the patient's bones. Unlike the imaging of soft tissues and the outer surfaces of skeletal structures, the differentiation of vasculature within skeletal structures can be particularly challenging. Similar issues can be encountered with imaging of fluid and blood flows within and/or adjacent to bones. Moreover, particular locations within a given bony structure may be difficult to image, owing at least in part to the density and orientation of relevant and/or adjacent structures.

In an initial step, anatomical image data is obtained of an individual patient's anatomy. This image data can be derive from a wide variety of sources, including MRA (magnetic resonance angiography), MRI (magnetic resonance imaging), x-ray imaging, cone beam CT, digital tomosynthesis, and ultrasound, CT scans or PET or SPECT scans. Desirably, image data is obtained that includes the patient's biological structure(s) of interest, which in one exemplary embodiment includes anatomical structures of a patient's lower extremity. For example, pixel or voxel data from one or more radiographic or tomographic images of the patient's anatomy can be obtained using magnetic resonance angiography. Other imaging modalities known in the art such as MRI, ultrasound, laser imaging, PET, SPECT, radiography including digital radiography, digital tomosynthesis or cone beam CT can be used. Contrast enhanced imaging can be employed, if desired.

Desirably, one or more of the pixels or voxels of the image data are converted into one or a set of values. For example, a single pixel/voxel or a group of pixel/voxels can be converted to coordinate values, such as a point in a 2-D or 3-D coordinate system. The set of values could also include values corresponding to the pixel/voxel intensity or relative grayscale color. Moreover, the set of values could include information about neighboring pixels or voxels, such as information that corresponds to a relative intensity or grayscale color and or information corresponding to a relative position.

The image data can be segmented, partitioned or otherwise altered into multiple segments or superpixels. The goal of segmentation is to simplify and change the representation of an image into something that is more meaningful and easy to identify. Image segmentation can be used to locate features and boundaries, such as data corresponding to a particular biological feature of interest. For example, the image data can be used to identify edges of structural features of the relevant anatomy, such as surface outlines of a bony protrusion, a tissue margin and/or a joint surface. In various imaging systems, a distinctive transition in color intensity or grayscale at a structure's surface can be used to identify pixels, voxels, corresponding data points, a continuous line, and/or surface data representing the surface of the biological structure. These steps can be performed automatically (for example, by a computer program operator function) or manually (for example, by a clinician or technician), or by various combinations of the two.

If desired, segmented data can be combined, such as in a single image including selected segmented and/or identified reference points (e.g., derived from pixels or voxels) and/or other data that can be combined to create a line representing a surface outline of a biological structure. In various embodiments, segmented and/or selected data from multiple 2D image slices can be combined to create a 3D representation of the biological structure. Depending upon the in-plane resolution and slice thickness (which can together define a voxel size, if desired), the field of view, the matrix size and the slice gap, the images can be combined to form a 3D data set, from which the 3D representation of the biological structure can be obtained. In various embodiments, a computer program could be used to load and view 2D images or 3D images could view multiple 2D images as one or more views of 3D image stacks. A series of image slices along one axis and a series of image slices along a second, non-parallel axis could be viewed as separate stacks of 2D images. Stacks of images could result from separate image scans (which can include the use of a single imaging modality along multiple reference planes as well as the sequential imaging of anatomy of interest using different imaging modalities along the same or different planes for each modality) or could be differing views or viewpoints of the same scan. In addition, any two or more images could be combined to provide a 3D image or image approximation.

In various embodiments, the 3D structure of an anatomical feature can be derived directly using a 3D segmentation technique, for example an active surface or active shape model algorithm or other model based or surface fitting algorithm. Alternatively, a 3D representation of the biological structure could be generated or manipulated (i.e., corrected or smoothed) by employing a 3D polygon surface, a subdivision surface or a parametric surface such as a non-uniform rational B-spline surface. Various methods are available for creating a parametric surface, which can include converting the 3D representation directly into a parametric surface by connecting data points to create a surface of polygons and applying rules for polygon curvatures, surface curvatures, and other features.

In one alternative embodiment, a template model could be applied to approximate and identify a biological feature or could be applied directly to an image data array. For example, an extremity template could be applied to an image data file and/or subsequently segmented image data. In applying a template model, the operator, user or the software itself could select one or more initial best fit template models. Template models of relevant anatomical structural features can be obtained from a library of models or other publicly available sources.

Obtained anatomical image data can include points, surfaces, landmarks and/or other features, which can collectively be referred to as “reference points.” In certain embodiments, the reference points can be selected and/or identified by an automated program or manually by an operator and used to identify an anatomical feature and/or region of interest. For example, reference points from an anatomical image of an extremity could be used to identify particular anatomical features of the extremity, such as the various bones, joints and relevant hard and/or soft tissue structures, which in turn can be used to identify one or more specific regions of interest of the image data for further analysis. If desired, reference points can be grouped to form reference structures and/or surfaces, including triangles, polygons, or more complex surfaces such as parametric or subdivision surfaces.

Once the appropriate anatomy is identified, one or more regions of interest in the image data will desirably be identified. For example, if an extremity structure can be identified from the segmented data, the relative location of a relevant vascular and/or microvascular circulation within the extremity can be identified and assigned or “bounded” as one or more regions of interest (ROI) of the image data. This ROI can be analyzed in a variety of ways, and the analysis results can be compared to a defined value and/or standard (and/or can be displayed and/or assessed using a value “map” of RI(s) in 2D or 3D space) and utilized to diagnose, assess and/or quantify pathology. If desired, the analysis and diagnosis can be used as guidance for treating the patient.

Once sufficient image data has been obtained, and has been sufficiently segmented and identified as relevant, it can be analyzed in a variety of ways. The data may also be processed, enhanced, filtered and/or otherwise modified in a variety of ways to desirably enhanced the detection and identification of various values of interest, which in various embodiments may include structural and/or functional qualities of microvasculature and capillaries (i.e., structural, functional, perfusive and/or other values). While various embodiments described herein include the analysis and assessment of various skin or other tissue pathologies, it should be understood that the techniques and treatments described herein can be applied with equal utility to virtually any anatomical feature, including bones and/or other joints of a human or animal body, as well as to other tissues and organs.

Various embodiments described herein include the use of a variety of image data types, and a variety of analysis approaches to the imaged data, which can be utilized in varying ways to identify vascular/microvascular perfusion deficiencies and/or diffusion insufficiencies adjacent to a skin wound or other region of interest. Relevant image data and analysis particularly useful in various embodiments disclosed herein can include one or more of the following (each of which may be utilized alone or in any combinations thereof): (1) analysis of the structure of soft tissues, including relevant vasculature and micro-vasculature structure and composition, (2) analysis of the flow and/or flowpaths of blood and/or other nutrients and wastes, and (3) analysis of nutrients, waste metabolites and/or “markers” entering and/or exiting the tissue of interest, which could include collection and analysis of blood or other fluids exiting the targeted tissue region or non-invasive imaging assessment of the presence of such nutrient/markers in the vascular system and/or relevant tissues of the integumentary system.

As more particularly explained in various portions of this disclosure, one unusual feature of a given skin region is that it may be capable of receiving nutrition via diffusion from surrounding adjacent tissue regions in a variety of directions. This potential for multi-axial sources and related vascular flows that can deliver some level of nutrients to skin tissues can potentially complicate the analysis, assessment and treatment of vascular hypoperfusion and deficient diffusive nutrient flow. In various embodiments, modeling and/or analysis of such multi-axial source flows could be accommodated in the imaging and analysis of a given extremity and/or skin region.

In various exemplary embodiments, the relevant features of vasculature and tissue structures adjacent to a targeted skin region of interest can be desirably imaged, identified and analyzed. Because a skin region can potentially receive nutrition from a variety of source locations, a nutritional deficiency in one individual source direction might not necessarily result in significant degradation of tissue health. For example, a skin region experiencing a nutritional deficiency via a hypoperfused vascular supply might be able to obtain some or all of its needed nutrition from one or more adjacent skin regions, possibly including various combinations of cephalad, caudal, medial and/or lateral adjacent tissues. However, where sufficient lack of vascular and/or nutritional flow in the region of interest occurs, or where a significant tissue degradation demands additional nutritional support to facilitate healing of the skin, the diagnosis may mandate some form of angiogenic (or other) treatment. In various embodiments, the effects of perfusion and/or diffusion and/or other nutrition/waste pathways relative to the skin tissues may be imaged, quantified and analyzed in the various analytical and treatment regimens described herein.

In various embodiments, three-dimensional (3D) imaging data of a patient's anatomical structures immediately adjacent to the tissue region of interest can be obtained and analyzed. In at least one desirable embodiment, the 3D data will include information regarding the anatomical structure of the skin and related tissues to a depth of at least 3 to 5 mm from the skin surface (a “Region of Interest”). In addition, the 3D data will desirably be of a sufficient resolution to differentiate and identify the relevant vasculature within this Region of Interest, including the various features of the capillary beds and optionally the arterioles, venules and/or other microstructure therein. In various embodiments, the data may alternatively and/or in addition comprise analysis of the perfusion of blood and/or other nutrients and wastes and/or analysis of nutrients. In a similar manner, waste metabolites and/or “markers” entering and/or exiting the tissues might be imaged and analyzed. In addition, since the ROI (region of interest) could be placed anywhere on an extremity and/or other body portion, it could be possible to image numerous areas of potential risk and/or concern to determine whether angiogenic treatments of a plurality of “blockages” and/or other potential ischemic regions might be appropriate and/or warranted.

The typical degenerative process of a skin wound can be a slow, continuous process. However, quantitative measurements such as those described herein may delineate subtle changes that can be clinically relevant. As precursor to morphologic changes, such functional measurements may be especially valuable during the early phases of the degeneration process where no morphological change is expected or anticipated to be present in the tissues, or at least not at an easily detectable level. Ideally, any potential quantitative, functional measurement reflecting the dynamic degenerative stages can be evaluated in correlation to an established quantification method. Where such subtle changes can be identified and/or detected, they can also be treated with several of the methods described herein (as well as others that may be developed in the future), which may slow, prevent and/or reverse the onset of later stages of tissue degeneration.

In a similar manner, the healing process of a skin wound can occur in a slow, continuous process. Desirably, once treatment begins, quantitative measurements such as those described herein may delineate subtle changes that can be clinically relevant. For example, functional measurements may be especially valuable during the early phases of the healing process, where morphological changes are not easily detectable in the tissues. Where such changes can be detected, it may indicate that the treatment regime is effective and the healing process has begun. Conversely, if no morphological changes are seen, this might indicate that the treatment is ineffective, which may mandate a differing treatment and/or different/increased dosing regimen. In various embodiments, quantitative measurements such as those described herein may be used to “follow” the wound healing process at almost any phase following appropriate treatment.

A significant advantage in the employment of the imaging and assessment systems described herein is the ability to measure and assess small changes in various tissue structures over time in a highly accurate manner. This facilitates the identification and/or quantification of subtle metabolic and structural changes in one or more tissue “regions of interest.” Until the approaches described herein were developed, such subtle changes were often difficult and/or impossible to detect, which made it commensurately difficult to determine if a given non-surgical and/or surgical intervention and/or treatment would be particularly effective in treating and/or ameliorating a degenerative tissue condition. By employing the various systems and methods described herein, however, it becomes a relatively straightforward process to assess and quantify the various advantages and/or disadvantages a given clinical intervention provides to treatment of a given tissue region. Measuring the nutritional and metabolic parameters of tissues before and after treatment can offer an evidence-based approach to analyzing the outcome, which can be of significant value to the assessment of existing tissue treatment regimens as well as those to be developed in the future.

In some embodiments, specific grades of tissue degeneration can be chosen for treatment, or a relative measure between similar tissues and/or microvasculature perfusion values at various skin regions of interest of a single patient may be compared to identify one or more areas having unusual and/or atypical values, which may indicate need for treatment and/or further assessment.

In various embodiments, assessment of perfusion can be performed, followed by therapy that increases the rate of perfusion, followed by a subsequent assessment of perfusion so as to identify the ideal conditions for stimulation of perfusion on an individualized basis. In other embodiments, assessment of perfusion may be performed to identify and/or evaluate areas that may require angiogenic treatment to prevent and/or alleviate skin breakdown and subsequent chronic wounds. In such instances, image data might further be useful in guiding such treatments, such as by percutaneous administration of angiogenic factors, via an image-guided approach. If desired, angiogenic factors could be injected into a targeted anatomical area, although in other embodiments instillation (i.e., subcutaneous injection and subsequent draining or withdrawal after a desired amount of time within the anatomy) could be accomplished.

For a typical region of skin tissue, the vasculature and/or microvasculature adjacent to the region will often not be constant across the entire region, but rather can vary depending upon the relative location of the various vascular sources supplying nutrients to the region. Skin tissues closer to vascular supply sources are more likely to receive sufficient oxygen and nutrition than skin tissues further from such sources. Moreover, various factors can affect the distribution and/or integrity of the microvasculature, including age-related diminishment of skin capillaries and/or various diseases.

Various embodiments described herein include the employment of 2-dimensional and/or 3-dimensional analysis of the vascular circulation and/or microcirculation directly adjacent to one or more tissue regions of interest. This may include localized analysis and/or “weighting” of the circulation/microcirculation measurements in different areas of the body, including in one or more extremities. In addition, multi-parametric analysis can provide a method to assess multiple aspects of a pathologic process that may exist simultaneously. This technique can provide important information on the degree of perfusion and/or hypo-perfusion of the tissues and well as quantify actual and/or potential tissue degeneration.

As previously noted, an unusual feature of the integumentary system is that the skin is typically capable of receiving nutrition via diffusion from surrounding peripheral tissues in almost any direction. This peripheral vascular flow, which can typically deliver nutrients to a given skin location from “any point of the compass,” has a potential to complicate the analysis, assessment and treatment of vascular hypoperfusion and deficient diffusive nutrient flow to a specific skin region. Because the skin can potentially receive nutrients from many sources, a deficiency in one specific direction and/or region may not have a significant clinical consequence mandating immediate treatment. In order to assess such considerations, however, it is desirous to obtain image data for the surrounding vasculature and/or microvasculature adjacent to a targeted skin location.

In one exemplary embodiment for imaging a microvascular network, an initial dynamic MR Perfusion technique can utilize a more pronounced temporal resolution with less spatial resolution and demonstrate rapid flow in the vasculature with a rapid wash-out rate. For example, modification of pulse sequences for a higher spatial resolution (smaller voxel size with a sub-millimeter in-plane resolution) at a cost of lower temporal resolution (a longer sampling time for each dynamic frame) can localize enhancements around microvasculature of interest that may not be evident from the data provided by a higher temporal resolution DCE-MRI (at a cost of lower spatial resolution) In addition, this technique can display time-course data (dynamic data) that is more associated with a discontinuous (or porous) capillary network. It is believed that this type of capillary is utilized by the hematopoietic functions of various tissues to a greater extent (allowing large cells to migrate from the intravascular and extravascular compartments). However, where a modified DCMRI (dynamic contrast magnetic resonance imaging) perfusion study is utilized, a significantly greater spatial resolution (and less temporal) protocol can be achieved, and this approach demonstrates significantly greater detail at the microvasculature level. Utilizing such a modified imaging protocol, it is possible to successfully image a tissue capillary network that can provide useful image data to be analyzed in various of the embodiments described herein. Such imaging parameters can allow detection of a time-course data consistent with a function of nutrient exchange.

In various embodiments, scans can be created demonstrating significant dynamic tissue perfusion that can be quantified with resolution up to 1 mm “in plane” and showing time course data that is consistent with capillaries that are continuous (no pores).

It is believed that various imaging and analysis approaches to the imaged data can be utilized in varying ways to identify vascular deficiencies and/or diffusion insufficiencies adjacent to a tissue region of interest. In various embodiments, image data can be acquired that reflects perfusion of blood in and/or proximate to various tissue layers. Where proper imaging modalities are used, and combinations of such data obtained from differing imaging modalities combined in a desired manner, image data can be acquired that reflects the flow and/or flowpaths of blood and/or other nutrients in various tissue regions In various alternative embodiments, image data can be acquired that reflects the structural composition of the vasculature and/or microvasculature, including reconstruction of the various circulatory and microcirculatory paths proximate a tissue region of interest. Another approach could include imaging and/or analysis of waste metabolites or “markers” exiting the tissues of interest, which may include collection and analysis of blood or other fluids exiting a wound area or non-invasive imaging assessment of the presence of such waste “markers” in the vascular system (i.e., taken from the local region and/or downstream regions, if desired) and/or relevant tissues.

In various embodiments described herein, anatomical image data from a patient can be obtained and the image data for one or more tissue regions of interest can be analyzed for the presence and/or likelihood of ischemia. For example, the image data of a microvascular network proximate to a skin wound or ulcer can be selected and analyzed using various techniques described herein, and the resulting analysis queried for the presence of hypoperfusion.

Numerous methods are known in the art that could potentially be used to identify areas of hypoperfusion. These methods can include MR-based techniques such as diffusion-weighted imaging, T2 and T1-weighted anatomical magnetic resonance imaging (MRI), diffusion tensor imaging (DTI), magnetic resonance spectroscopy (MRS), T1ρ weighted MRI, dynamic contrast-enhances MRI (DCE-MRI), T2 relaxometry MRI, CT-scan (computed tomography scan), and provocative discography. Diffusion-weighted imaging can provide quantitative analysis of tissue degeneration and early changes over time as previously described. T1ρ MRI can be used to measure proteoglycan content. Any of these techniques may be used alone or in combination to diagnose dermal and/or sub-dermal ischemia as described herein.

In one particular embodiment, the area of hypoperfusion could be identified using technetium-99m Sestamibi in conjunction with single photon emission computed tomography (SPECT) imaging. This radiolabelled lipophilic cation can be injected intravenously at concentrations ranging from 200-1790 MBq, more preferably 500-1000 MBq, and even more preferable at approximately 750 MBq. Imaging can be performed with a gamma camera and absorption/perfusion quantified using various software packages known to one skilled in the art. In some embodiments, to attain appropriate images, the camera may be rotated to a plurality of angles, up to and including rotation of 360 degrees.

In other embodiments, various means of detecting hypoperfusion could be employed, for example, PET-CT (positron emission tomography—computed tomography), DCE-MRI, and, for example, fluorescent peptide-based methodologies.

Perfusion and/or Diffusion Imaging

In various embodiments of the invention, diffusion studies (Diffusion Weighted images or DWI) can be performed for analyzing the diffusion characteristics of the integumentary system and potentially correlating it to vascular hypoperfusion, microvascular hypoperfusion and/or arterial or venous degeneration, occlusion, blockage or stenosis. The use of Diffusion Weighted Images (DWI) can potentially help to analyze the diffusion characteristics of the microvasculature and related integumentary system and correlating it with skin degeneration and/or healing abnormalities. Solute transfer into the upper layers of the skin can be dependent upon the concentration of the solute at the microvascular level (which can be correlated with vascular perfusion) and the diffusion characteristics of the intervening anatomical layers. Abnormalities in diffusion contribute to skin degeneration and healing abnormalities. Analyzing diffusion properties among various patient populations (as well as normal controls) may lead to data that can contribute to an ischemic condition disease diagnosis.

In various other embodiments, perfusion studies could be performed using non-invasive and/or minimally-invasive imaging methods such as Dynamic Contrast Enhanced MR Imaging for analysis of perfusion of the systemic/extremity vasculature and/or localized microvasculature of soft and/or hard tissues. For example, one method could include using a 1.5 Tesla scanner to evaluate a potential for ischemia-related cell damage. However, higher powered imaging equipment, such as 3 Tesla or higher scanners, may significantly improve the accuracy and resolution of image data, which can be particularly useful in imaging and assessing the microcirculation proximate an area of interest. If desired, imaging parameters for a 3 Tesla scanner could be utilized to facilitate the acquisition of such useful image data. Other systems could be used, if desired, including those that employ the use of high-field magnets due to their higher SNR (signal to noise) and CNR (contrast to noise) ratios in comparison to lower strength magnets. Such systems could potentially allow a lower dose of contrast material to be delivered to the patient yet allow generation of an equivalent image quality to those of lower-field magnets with a higher dose of contrast. Such a system may also permit the use of serial (multiple) bolus contrast injection for multiple scanning sequences of the patient, potentially using different scanning techniques and/or modalities. The use of higher strength systems, including those with 7-10 Tesla magnets, may improve the resolution and accuracy of scanning, including the potential to directly image the microvasculature and/or vascular buds. If different imaging techniques are to be employed, it may be desirous to complete any non-contrast imaging initially, and then subsequently perform contrast-assisted imaging, to reduce the potential for imaging errors and/or artifacts caused by the contrast and/or its remnants during the non-contrast imaging techniques.

For imaging protocols in one exemplary embodiment, the following could be used in conjunction with a Philips Achieva 3T system: 330 mm×300 mm FOV and a 6-element SENSE torso RF coil. The imaging session could be started with the perfusion scan following the standard calibration scans. A 3D FFE sequence with TR/TE=3.5 ms/1.5 ms, SENSE factor: 2.5(AP), 2(RL), flip angle=30°, with dynamic scan time of 2.9 s can be used and 7 slices in sagittal orientation with 6 mm thickness and 1.9 mm×1.9 mm pixel size could be acquired. A total of 114 volumes can be collected, 2 of them before contrast injection. After the dynamic scans, T1 weighted anatomical images in sagittal plane can be collected using a TSE sequence with 0.5×0.5×3 mm3 voxel size. 14 slices cover the same volume as dynamic scans. TR/TE=900 ms/10 ms, flip angle=90°. This can be followed by a T2 weighted scan having identical geometry to T1 scans and TR/TE=2940 ms/120 ms, flip angle=90°. Finally, contrast-enhanced angiography scans can be collected. Contrast bolus arrival can be observed real-time using a single, 50 mm thick coronal slice using FFE sequence in dynamic mode, collecting images every 0.5 s. Once the contrast arrives in the relevant peripheral vessel, actual 3D angiography scans can be started by the operator immediately. TR/TE=5.1 ms/1.78 ms, voxel size=0.8*0.8*1.5 mm3, with SENSE factor=4 can be used to acquire 50 coronal slices. Peripheral/segmental vessels on MRA can be graded as occluded, stenotic or open, if desired. ROI-averaged time course data (from regional tissues and/or dermal microvasculature proximate to the skin wound) can be converted into a fractional enhancement time course and analyzed using a compartmental model (Larsson, et. al. MRM 35:716-726, 1996; Workie, et. al. MRI, 1201-1210, 2004). The model fitting can result in 6 parameters: Ktrans′ (apparent volume transfer constant), kep (rate constant), Vp′ (apparent fractional plasma volume), E (extraction fraction), tlag (arrival time of tracer in the ROI) and baseline.

In one alternative exemplary embodiment, a high spatial resolution version of DCE-MRI could include a 3D gradient echo-based sequence with TR/TE=3.4/1.2 (ms), flip-angle=30 (degree), reconstructed voxel-size=0.8×0.8×3 (mm), temporal-resolution (or dynamic scan time)=36.4 (sec) w/22 dynamic frames (volumes). The entire bolus of contrast could be utilized for the DCE-MRI, which may be preferable for this embodiment, or the contrast can be given in two boluses, one for DCE-MRI and one for MRA. Other non-contrast scans (i.e., T1 and T2w) could employ the same or similar acquisition parameters as described above, with non-contrast imaging desirably preceding contrast-assisted imaging where possible.

In various embodiments, perfusion measurement and assessment via DCE-MRI or other imaging modalities could be performed at the capillary level, especially in terms of ‘high spatial resolution’ type DCE-MRI. Such scans could potentially differentiate where contrast material were to “leak out” and accumulate in extravascular, extracellular-matrix (ECM) space, and could also measure where and/or if the contrast material eventually “cleared out” of the ECM, given a sufficient scan duration. This could significantly improve the ability to image and resolve the actual blood and/or nutrient flow as compared to imaging of the exchange between the ‘vascular’ space (capillary) of interest and the ECM space (which may be of lesser interest, depending upon the surgeon's preference). For example, if the imaged contrast-material were of the intravascular type (i.e., it does not easily leak out from ‘normal’ capillaries), the level of detectable signal ‘enhancement’ that could be measured during DCE-MRI scanning might be very low because of the relatively small percentage that might be considered as ‘vascular space’ in a typical imaging voxel-size for most biological tissue.

Similar differentiation of such extravascular and/or extracellular presence of contrast (i.e., Omniscan: Gd-DTPA-BMA) could be possible with contrast material used in other imaging modalities, including routine imaging modalities such as CE-MRI. If desired, the assessment of blood supply or flow into such capillary networks could also be evaluated ‘up-stream’ (i.e., in larger arteries) and/or “downstream” as part of the imaging and assessment process herein.

In various embodiments, the use of combinations of CE-MRA and DCE-MRI in the same MRI or in a sequential scanning session could be performed. While CE-MRA can be combined w/CE-MRI, CE-MRA may not provide a desired level of ‘quantitative’ information to the surgeon as compared to an equivalent DCE-MRI imaging session. In such situations, the use of higher strength magnet systems could desirably allow the injection of reduced doses of contrast for such serial imaging, thereby allowing for the collection of greater amounts and/or resolutions of data (which can be combined post-imaging, if desired) than that of a single imaging modality alone.

In various alternative embodiments, the use of intravascular contrast material might be preferred, as this material may not lend itself to diffusion from the vasculature, but such use could also be limited in its imaging of diffusive patterns from the capillary network. In contrast, the use of easily diffusing contrast, in combination with the ability to differentiate leaking contrast versus intravascular contrast, could potentially facilitate direct imaging of flow patterns and vasculature structure, while ignoring or discounting such contrast potentially in the (ECM) space.

### MR Spectroscopy and Other Studies

A loss of perfusion in the dermal and/or sub-dermal levels can result in less oxygen available for diffusion across into the skin. Since simple diffusion appears to be the primary mechanism for solute transport to the skin and not a pumping action, the oxygen concentration in the various dermal and/or sub-dermal levels can be critical. Loss of oxygen (hypoxia) results in a shutdown in matrix production and resulting poor matrix repair and maintenance. High field strength spectroscopy (which may desirably be of at least 3 Tesla strength, although lesser or greater strengths may be used with varying levels of utility) may be extremely important in the delineation of metabolic abnormalities associated with ischemia within the skin. It has been demonstrated that lactate levels and/or other metabolic waste markers can be elevated in tissues dependent upon anaerobic metabolism. Therefore, lactate could be used as a biochemical marker signifying a skin region that is “stressed” and at risk. In addition, low pH (associated with high lactate) has been demonstrated to be a biochemical mediator of pain in various tissues. Other useful markers that may correlate with ischemia/hypoxia and the painful, degenerative tissues include, but are not limited to, determination of 31P levels as an indicator of energy level and water content.

In one exemplary embodiment, proteoglycan quantification could be measured in vivo using a Mill imaging technique called T1rho (T1ρ) sequence. Just as ADC value (ADC-mapping) can be a quantitative outcome of diffusion-weighted imaging (DWI), T1ρ relaxation time (T1ρ mapping) can be an outcome of T1ρ weighted imaging wherein the relaxation time is shown to be directly correlated to PG (proteoglycans) content. Relevant data obtained could be used by a clinician to identify the hallmarks of tissue degeneration, including the loss of proteoglycans, water, collagen and/or other changes in the tissue matrix, and recommend further analysis, imaging and/or treatment including the various techniques described herein.

### Structural Imaging and Modeling

In various embodiments, non-invasive imaging and data collection can be utilized to obtain a two or three dimensional model of the anatomy proximate to the skin wound or ulcer, which can include underlying hard tissues (i.e., bone) as well as related soft and/or connective tissues. In various embodiments, it may be advantageous to image and model some portion of an extremity of the patient, especially where one or more skin wounds or ulcers requiring treatment have occurred on a load-bearing extremity such as the bottom of the foot. In such a case, it may be desirable to image the entire lower surface of the foot as described herein to obtain and/or derive a three-dimensional model of the underlying bony support structure and/or all related soft tissues of the foot. Once such data is obtained, it could be utilized for a variety of assessment and/or treatment functions, including as a guide to model a prosthesis for protection and/or “offloading” of one or more of the skin wounds and/or ulcers.

### Combination Imaging Strategies

In various embodiments, combinations of imaging strategies and/or methodologies can be employed to collect image data. In various embodiments, the various image data types obtained can be used for generation of algorithms to include/exclude patients and identify “at risk” tissues, including those suffering from vascular or diffusive deficiencies and/or potential structural deficits. Combining imaging studies may provide important insight into the description of heretofore unknown vascular diseases of various tissues. In one embodiment, the clinician treating patients may recommend longitudinal DCE-MRI for analysis of tissue perfusion along with T1ρ and/or ADC. These studies can show a correlation of accelerated detrimental changes within the skin tissues that, coupled with an association with hypoperfusion and/or ischemia may satisfy one or more inclusion criteria for treatment of the hypoperfused tissue region with angiogenesis. This static image combination could provide important clinical information that leads to medically necessary treatment protocols. In addition, combinations of image techniques might be utilized—i.e., multiple different imaging modalities within a short time period and/or multiple imaging modalities over time using complimentary, serial modalities for analysis. A clinical treatment plan could also be developed based upon the results of the multiple/serial imaging acquisitions.

In various embodiments, data could be collected from control and/or experimental subjects to ascertain an “ischemic index” of the dermal and/or sub-dermal microvasculature, which could desirably be applied to future assessments of ischemic/hypoxic tissue disease. The data can be correlated with the degree of skin/wound degeneration and potential areas of arterial and/or venous stenosis. Since perfusion analysis can potentially measure the amount of blood supply coursing through the extremity and microvasculature thereof, and therefore can be relevant to the amount of nutrition available for the skin, this value can be important in developing treatment schemes based on improving the blood supply to the skin.

If desired, one embodiment of modeling and analysis of the vasculature and/or microvasculature could include the step of structural modeling of the vessel anatomy and/or perfusive blood flow in the imaged extremity and/or anatomy, which can include simulation modeling of anticipated treatment(s) and/or outcomes based on a variety of treatment regimes, including the use of angiogenic treatments such as described herein. For example, the perfusion data from an imaged region might show a region of vasculature and/or microvasculature underlying a skin region of interest that is sparsely populated with vessels and/or involves lower-than-normal flowrates. It may be desirous to modify the model of the region to incorporate vasculature and/or capillaries that are more densely distributed, and/or vessels growing more proximate and/or closer to the area of skin damage, to determine whether an angiogenic treatment might be desired and/or appropriate to the skin region. In various embodiments, the modeling of capillaries, especially those in a highly structured tissue, could be approximated using an array of cylinders with nearly uniform spacing. Desirably, the model could be utilized to identify areas where angiogenic treatment could be particularly advantageous, as well as identify where drug delivery might be improved by reducing the distance to the nearest vessel and/or by ensuring that blood flow is sufficiently strong and/or uniform in the vascular/micro-vascular network so that each vessel is well-perfused.

### Treatment

Once an area of deficient nutrition, vascular perfusion and/or other anatomy of interest has been identified and analyzed, it may be desirous to treat the area (or other relevant anatomical structures) in an attempt to slow, halt and/or reverse the progression of diseases that may be present and/or develop in the future. In various embodiments, the treatments described herein may have particular utility in preventing and/or reducing skin breakdown in various patients, including in “high-risk” groups such as diabetics.

As used herein, the terms “treating,” “treatment,” “therapeutic,” or “therapy” do not necessarily mean total cure or abolition of the disease or condition. Any alleviation of any undesired signs or symptoms of a disease or condition, to any extent, can be considered treatment and/or therapy. It is entirely possible that “treatment” consists of a temporary improvement of the microvasculature and/or vasculature supporting the skin region of interest, with additional repeated treatments required over time to continue the regenerative process. In addition, asymptomatic hypoperfusion may be the focus of treatment utilizing angiogenesis. Furthermore, treatment may include acts that may worsen the patient's overall feeling of well-being or appearance. Various embodiments described herein include desirably restoring perfusion to the anatomy adjacent a skin region (as described herein), which may ultimately provide sufficient diffusive nutrient and waste flow to maintain a minimum or acceptable nutrition level and reverse, reduce and/or slow the degradative cascade of skin and/or various tissues.

Once an area of hypoperfusion or other deficit is identified as described herein, the patient may be diagnosed with hypoxic and/or ischemic tissue disease, and various embodiments include the induction of neovascularization so as to enhance localized perfusion to the area of need. In the case of a diagnosis of ischemic vasculature and/or microvasculature relevant to tissues of interest, various embodiments include the induction of neovascularization so as to enhance localized perfusion to the area of need. If desired, quantitative measurements of diffusion weighted imaging and Apparent Diffusion Coefficient or ADC can be utilized to identify “at risk” tissues (which could also include determining the degree of such hypoperfusion and/or utilizing such information to verify the identity of an “at risk” tissue region). Alternatively, or in addition to such ADC measurement and assessment, tissue integrity imaging using either Ultra-short TE (UTE) imaging, assessment of proteoglycan content of various tissues using T1ρ magnetic resonance imaging quantification, measurement of lactate removal by a “metabolite imaging” technique such as Magnetic Resonance Spectroscopy (or 1H-MRS) or phosphorus scanning such as 31P-MRS for pH or bioenergenic metabolism of the tissues, or similar assessment methodologies could be employed. In other embodiments, various combinations of the above-reference data could be combined with tissue vascularity and any information regarding the change in the symptoms and other clinical factors of the skin or related anatomy to define the medical necessity for angiogenic treatment. The totality of these imaging modalities can be summed up by the process of imaging the entire nutrient delivery pathway to the skin region(s) of interest. At each level, nutrient delivery has the potential to be halted and the tissue integrity and bioenergetics affected. Measuring the level of occlusion and/or blockage and its resultant effect on the skin can potentially be accomplished using any combination of one or more imaging modalities, where tissue perfusion can be measured with DCE-MRI, tissue integrity and diffusion characteristics analyzed with T1ρ and ADC, tissue integrity quantified with ultrashort time to echo MRI (or some other integrity scanning modality) and cellular metabolism measured with some form of molecular imaging such as lactate or sodium.

In various embodiments, 2D and/or 3D imaging studies could be employed to define the specific and/or localized areas of the tissues and/or vasculature/micro-vasculature that could be best treated with angiogenesis. If perfusion analysis of various skin regions in an extremity or other patient anatomy appeared relatively normal relative to a desired imaging quantifier and/or assessment, and other skin regions appeared “at risk”, one potential treatment approach could be to provide an angiogenic treatment (i.e., injection and/or topical application) within and/or proximate to the “at risk” area. In alternative embodiments, it may be desirous to treat the “normal” area in an attempt to improve perfusion and/or prevent degradation in that level/area. Desirably, a combination of such treatments will restore and/or regenerate the normal capillaries of one or both areas (or at least improve such vascularity in one or more areas) and produce resulting improvements in perfusion and/or nutrient/waste delivery/removal.

In various embodiments, an assessment can be performed on a patient to identify “at risk” skin regions, and then a treatment plan can be created so as to avoid wounding and/or damaging those at risk areas. For example, if an assessment identified a left lower extremity of a surgical patient as at high risk of pressure sore formation during recovery, the treatment plan could include an instruction for a caregiver to move the patient's left leg at a more frequent interval than typical for a similar patient (or follow some other post-surgical recovery protocol). Similarly, the patient might be fitted for a compression sleeve or other pressure-relieving device on their left leg. If desired, one treatment regimen utilizing angiogenic factors could include instillation of an angiogenic factor (desirably for prevention of skin breakdown due to hypoperfusion and/or ischemia) in various situations.

In various embodiments, one anatomical location providing vascular support to a skin region of interest could show diminished perfusion, while a secondary region providing vascular support to the same skin region of interest could show normal perfusion. As skin regions can often obtain nutritional support from multiple source regions, it is possible that one region could be treated first and imaging measured for improvement before the other region might be treated.

In various embodiments, more than one skin region may be identified as “at risk” and in need of treatment. In this situation, imaging data may provide insight as to which skin region and/or supporting vessel network should be accessed for angiogenic treatment relevant to other selections, which in some situations may be a skin region most likely to be stressed in the future. Such a stress region could include the soles of one or both feet (i.e., for ambulation), a region likely to suffer from pressure sores during surgical recovery from a future scheduled operation, or a region likely to become injured after a surgical intervention that can affect vascular flow (i.e., taking a radial artery or saphenous vein graft for use in a coronary bypass operation). In such cases, a single angiogenic treatment may be used for the selected skin region and/or support vessel network, or multiple angiogenic treatments may be provided to multiple areas.

In various embodiments, an imaging study of a patient's extremities or other portions of the integumentary system (or portions thereof) may be performed, and analysis of the various vascular networks supporting tissues contained therein can be performed. Such studies can identify “at risk” tissues, vasculature and/or microvasculature, which may be diagnosed for treatment and/or further study at a later date. Where “at risk” tissues, vasculature and/or microvasculature may be identified, further studies may be performed, if desired.

In one exemplary embodiment of the invention, a patient can be diagnosed with hypoxic and/or ischemic tissue disease and treated by increasing localized perfusion through the use of angiogenesis induction. The process of new blood vessel formation (angiogenesis) can occur naturally, or be induced through various means, including but not limited to vasculogenesis, arteriogenesis, and angiogenesis. For the purpose of this invention, all three will be referred to as “angiogenesis”. Technically speaking, angiogenesis is associated with de novo capillary and arterial formation from pre-capillary arteries and arterioles and from post-capillary venules, is ischemia- and hypoxia-driven, and is associated with a 2-3 fold increase in blood flow. Angiogenesis can also include growth of or from existing capillaries.

Arteriogenesis is technically considered remodeling of pre-existing vascular channels (collaterals) or de novo artery formation, it can be stimulated by local changes in perfusion (shear stress), as well as cellular influx and proliferation, and associated with a 20-30 fold increase in blood flow. Vasculogenesis is technically considered on the one hand to encompass embryonic vascular development, and on the other hand to include de novo formation or remodeling of pre-existing vascular channels initiated by circulating vascular precursor cells; furthermore; it is considered to be ischemia and injury initiated. The term “angiogenesis” is meant to encompass all three technical terms.

Angiogenesis is known to occur physiologically during zygote implantation, embryogenesis, post-embryonic growth, and during tissue repair and remodeling. Pathologically, uncontrolled angiogenesis is associated with a variety of diseases such as macular degeneration, diabetic retinopathy, inflammation, including arthritis and psoriasis, and cancer. One common aspect of adult angiogenesis is tissue hypoxia. In situations of tissue expansion, cells are typically dependent on the microvasculature for nutrients and oxygen supply, as well as removal of metabolic waste products. Accordingly, during tissue growth, cells begin to “sense” a lack of oxygen. This triggers a cascade of events that culminates in angiogenesis. During pathological conditions, such as the conditions associated with hypoxic and/or ischemic tissue conditions, the lack of oxygen is induced through hypoperfusion. Said hypoperfusion may occur due to, for example, atherosclerosis. In some pathological conditions, the normal angiogenic response to hypoxia is absent or substantially diminished.

Although numerous methods of physiological stimulation of angiogenesis under hypoxia are known and thereby useful for the practice of the current invention, one of the most well characterized pathways involves activation of the Hypoxia Inducible Factor-1 (HIF-1), transcription factor. This protein is only functionally active as a heterodimer consisting of HIF-1α and HIF-1β, which are both basic helix-loop-helix proteins. While the latter is known to be relatively stable, the former has a half-life of less than 5 minutes under physiological conditions due to rapid proteasomal degradation by the oxygen sensitive von Hippel-Lindau (VHL) E3-ubiquitin ligase system. When cells experience hypoxia, HIF-1α half-life is increased since the degradation by VHL E3-ubiquitin ligase is dependent on proline hydroxylation, which requires molecular oxygen. Therefore, this protein modification plays a key role in mammalian oxygen sensing. Activation of this transcription factor leads to gene expression of numerous angiogenesis related genes such as VEGFs, FGF-2 response genes, notch signaling, and up regulation of stromal derived factor (SDF-1), which chemoattracts endothelial precursors during angiogenesis. There are numerous variations by which angiogenesis can occur; however, the basic steps involve remodeling of the extracellular matrix through matrix metalloproteases (MMPs), chemoattraction of either precursor endothelial cells or existing endothelial cells from an adjacent vessel, proliferation of the endothelial cells, tube formation and stabilization. Various embodiments described herein can include the transfection of genes encoding HIF-1 into areas of lumbar hypoperfusion in order to induce normalization of perfusion, or in some cases hyperperfusion in order to ameliorate or significantly treat hypoxic and/or ischemic tissue disease. Embodiments described herein relate to utilization of molecules that either induce the expression of HIF-1, or conversely delay the degradation of HIF-1 or components thereof including but not limited to FGFs.

In various embodiments, skin wounds, ulcers and/or other conditions can be treated by application and/or administration of a medical device that generates a periodic or continuous release of a composition which includes an angiogenic factor onto tissue, into tissue and/or into blood and/or fluid circulation so as to promote neoangiogenesis, and specifically, collateralization in area(s) proximal to the skin condition. In some embodiments, the composition might further include stem cells and/or other biological treatments, which might be used in conjunction with angiogenic factors prior to, during and/or subsequent to the employment of tissue grafts to repair or replace native tissues. If desired, such compositions could be used to prepare a patient's anatomical site for an intended tissue graft or surgical procedure, could be used to prepare the tissue graft for implantation, and/or could be used to treat the patient and/or tissue graft site after implantation.

If desired, the collection and analysis of imaging data and subsequent angiogenic treatments could be applied to virtually any anatomical area having one or more deficiencies and/or conditions that result in a large soft tissue defect (i.e., due to trauma, tumor or some other disease) that may require a combined surgical and angiogenic approach. If desired, the imaging data could be utilized to plan treatment of the soft tissue defect, including a proper skin closure procedure using reconstructive surgical techniques along with angiogenic treatment. The angiogenic factors could be provided alone or in combination with a scaffold with or without stem cells.

In a similar manner, the collection and analysis of imaging data and subsequent angiogenic treatments could be applied to virtually any anatomical area having one or more deficiencies and/or conditions that result in a large hard tissue defect (i.e., due to trauma, tumor or some other disease) that may require a combined surgical and angiogenic approach. For instance, an open tibia fracture with a poorly vascularized wound could be treated with various approaches described herein, including utilizing imaging data to plan a proper skin closure procedure using reconstructive surgical techniques along with angiogenic treatment. The angiogenic factors could be provided alone or in combination with a scaffold with or without stem cells.

In various embodiments, a medical device may include a reservoir, a slow release pump and/or some other supply device, which could include external devices as well as implantable indwelling or osmotic pumps or localized delivery systems. In various embodiments, the device may incorporate a polymer capable of slow release of materials incorporated therein.

In various embodiments, the composition delivered by the medical device contains not only a therapeutically sufficient concentration of a growth factor that stimulates angiogenesis, but also a chemotactic agent. Some growth factors, such as fibroblast growth factor 1 (FGF-1), are themselves chemotactic. The chemotactic agent recruits cells capable of causing or promoting angiogenesis. In some embodiments, a chemotactic agent such as stromal cell-derived factor 1 (SDF-1) could be included in the composition with the growth factor. In various embodiments, the composition delivered by the medical device may contain an anti-inflammatory agent at a concentration sufficient for inhibiting possible inflammatory reactions associated with neoangiogenesis, while at the same time not inhibiting collateral blood vessel formation. If desired, the various agents described herein could be combined with various scaffolds and scaffolding structures, as well as stem cells, which can include embryonic stem cells and/or adult stem cells, as desired.

In various embodiments, the treatment of patients could include various combination of active and passive treatment phases, wherein active treatment phases desirably induced a positive effect on healing of the patient's ulcer, which might even include improved healing effects in one of both of the active and/or passive phases. In many patients, a measureable extremity blood pressure level sufficient to provide a minimum level of nutrients and oxygen to the extremity is highly desirable, yet may not be absolutely necessary to realize some of the benefits of the various therapies described herein.

For example, in a leg, a Systolic toe blood pressure of at least 30 mm HG (≧30 mm Hg) may be preferred for treatment of a skin ulcer on that extremity. Moreover, depending upon the co-morbidities affecting a given patient, it may be preferred that only a single skin ulcer for each extremity be treated using an angiogenic formulation containing FGF-1. In such a case, the opportunity for angiogenic growth and tissue repair/regeneration might be maximized for the single ulcer, whereas multiple ulcers may reduce the effectiveness of the treatment. In other patients, multiple ulcers on a single and/or multiple limbs might be treated, as desired.

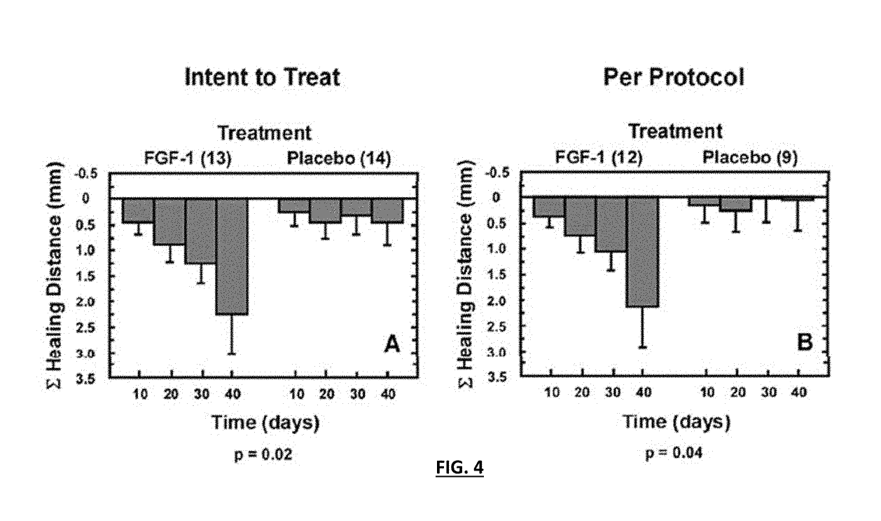

In another example, the topical application of an angiogenic compound, including FGF-1, to a skin ulcer of a patient suffering from chronic diabetic ulcers can significantly increase the rate of healing of the ulcer during the active treatment phase (as compare to a placebo or non-treatment group), but can potentially also induce significantly improved skin healing effects during a follow-on “non-treatment” phase (i.e., passive treatment phase) after cessation of the active treatment. One exemplary treatment regime for a series of patients suffering from diabetic chronic ulcers could comprise topical application of an angiogenic compound, including FGF-1, to the patients'skin ulcers at a frequency of three times a week, for a period of three weeks. The angiogenic compound can include dosing of 3 μg/cm² of FGF-1 for each patient. In one exemplary protocol involving human subjects, wounds treated with FGF-1 in this manner healed approximately 3 to 4 times faster than those treated with a corresponding placebo vehicle. Specifically, the wounds treated with FGF-1 healed by ingrowth from the original wound edge at an average rate of approximately 0.56 mm (over each period of 10 days) while the placebo group wounds only healed at a rate of approximately 0.125 mm (over each period of 10 days)—See FIG. 4. Moreover, while the active phase of ulcer treatment spanned only 3 weeks, the accelerated wound healing in the FGF-1 treated group continued at the accelerated rate for another 3 weeks (without additional application of the angiogenic compound), and at 6 weeks the rate of healing of the FGF-1 treated group reverted back to that of the placebo patients.

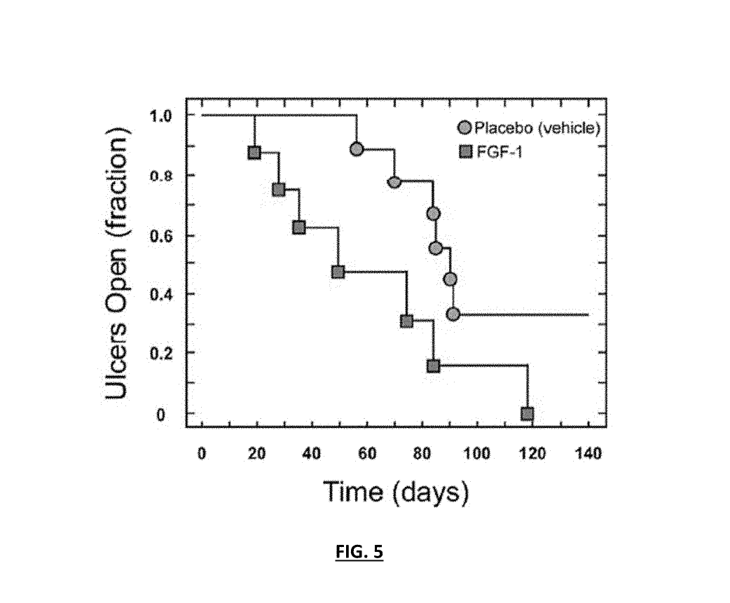

In another exemplary protocol involving human subjects suffering from chronic diabetic ulcers, wounds were treated with a topical application of an FGF-1 composition (i.e., the previously described 3 μg/cm² of FGF-1), which was applied to the ulcer and surrounding healthy tissue three times a week over a period of 20 weeks, and this treatment demonstrated superior wound healing to that of a placebo control. Under this protocol, the healing rates in the FGF-1-treated group were significantly greater (an average of 3 to 4 times faster) than in the vehicle placebo-treated group, with all the ulcers of the patients treated with FGF-1 closed and completely healed by the 17^{th} week, while ⅓ of the placebo group remained open and unhealed. Moreover, the FGF-1 treated ulcers healed approximately 40 days sooner than the ulcers of equivalent placebo patients. As best seen in FIG. 5, the ulcers of more than half of the FGF-1 treated patient group (i.e., 57%) had completely healed by day 50, whereas none of the placebo group ulcers had closed at that time.

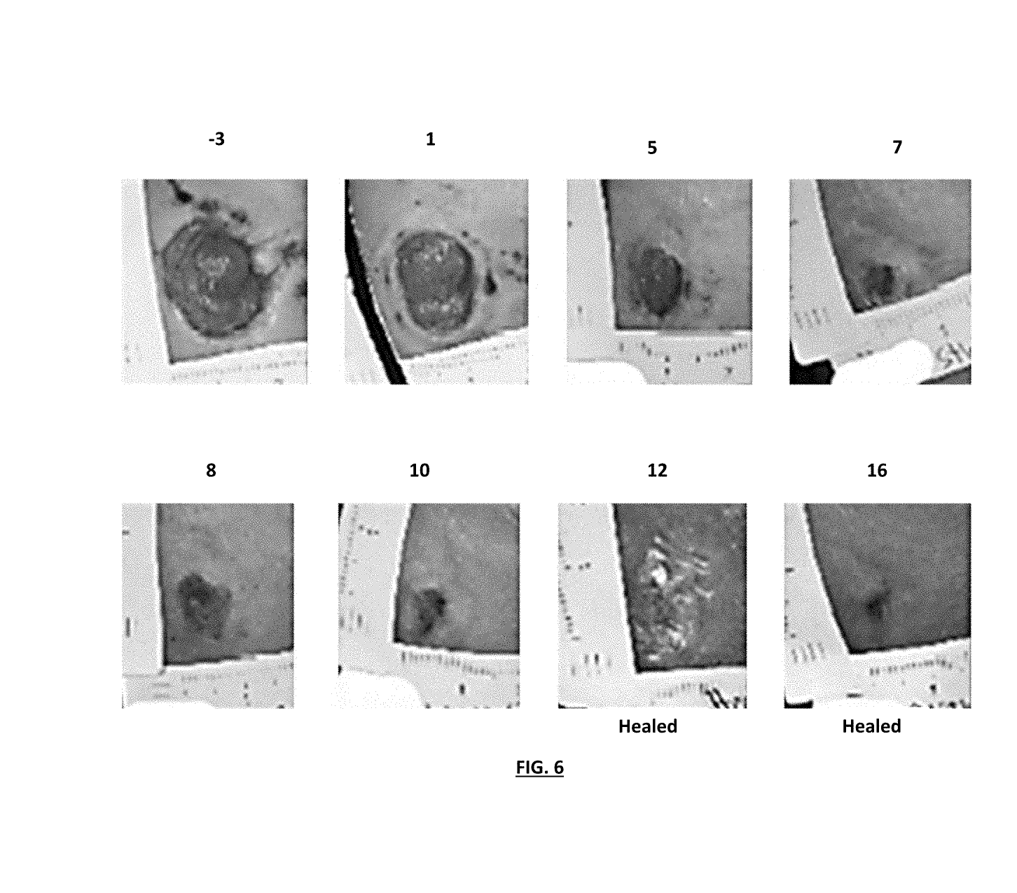

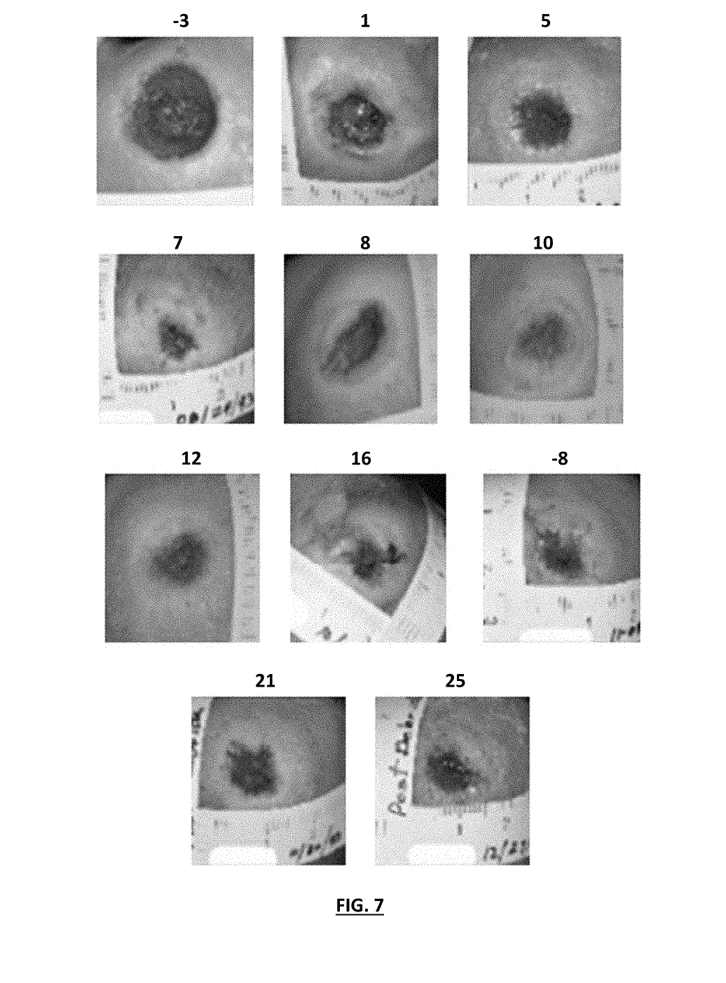

FIGS. 6 and 7 depict pictorial representations of a pair of equivalent skin ulcers of patients treated with a composition comprising FGF-1 (FIG. 6) and corresponding placebo doses (FIG. 7). In these Figures, the initial view labelled “-3” denotes the external visual condition of each ulcer at the beginning of a 3 week pre-treatment period, during which time the lack of appreciable closure served to identify a chronic non-healing diabetic ulcer in each patient. The label “1” denotes the initiation of treatment at week one. The FGF-1 treated ulcer was completely healed by day 74 of the treatment (shown pictorially in view “12” of FIG. 6). In contrast, the placebo-treated ulcer was unhealed at 12 weeks, remained an open wound at the end of 20 weeks of treatment, and was still not fully healed even at the end of an approximately one month follow-up observation period at the end of the study.

In another exemplary embodiment, an angiogenic composition comprising FGF-1 in a concentration of 10 mg/cm², which can be incorporated into a fibrin matrix, can be applied topically to a skin wound and/or surrounding external tissues, which desirably significantly accelerates the healing process of the skin wound and leads to significant improvement in healing, with complete epithelialization and minimal contraction, as compared to a natural, healthy healing response.

In various alternative embodiments, an angiogenic compound including FGF-1 might be injected and/or otherwise introduced beneath the external surface of the wound, such by injection via a hypodermic needle into a subsurface structure of the center of the wound, the wound margin and/or into underlying and/or adjacent healthy tissues. If desired, concurrent and/or alternating surface and subsurface treatments (including as previously described) could be undertaken.

In various treatments, the size, shape and/or condition of the skin ulcer might predispose the wound to a particular treatment or combination or treatments. For example, for a skin ulcer presenting less than an approximately 6 cm² external surface, a topical compound might be more appropriate for treatment. However, where the ulcer may be greater than approximately 6 cm², or where the skin ulcer includes damage to underlying bone, tendon or cartilage, it may be desirous to combine a surface treatment of the ulcer with one or more injections of a compound comprising FGF-1 into the ulcer, into the tissue region proximate to the margin between the ulcer and surrounding healthier tissues, and/or into healthier tissues surrounding the ulcer.

In a similar fashion, chronic wounds or ulcers, such as diabetic foot ulcers, or other wounds known to be of ischemic origin, could be treated in various combination approaches. For example, if cells, scaffolds, signaling proteins such as various growth factors, genes or any other tissue or synthetic transplantation were contemplated to be utilized in an area of ulcer on the diabetic foot or other area of anatomy that is suffering with a chronic ischemic wound, then proper pre-treatment ischemic analysis using various imaging modalities discussed herein might be utilized. If areas of ischemia were identified that required pre-treatment with angiogenic factors (prior to the previously mentioned transplantation or coverage procedure), then the proper dosage and administration of said angiogenic factors could be provided as a combination treatment.

In various embodiments, it may be desirous to treat an identified deficiency before significant tissue degeneration and/or damage has occurred, even where other adjacent tissues and/or vasculature appear to be providing normal nutrition and waste removal. For example, where a patient is initially diagnosed with diabetes, PAD or some other disease affecting the vasculature, where a patient will be undergoing surgery requiring a significant recovery period, or where long term bed rest is anticipated or becomes necessary, it could be useful to identify skin and related tissue locations or regions likely to suffer from the various vascular insufficiencies described herein. In many situations, the specific characteristics of the imaging data may demonstrate which vessels and/or tissue architecture may be susceptible to treatment versus other imaging data that shows capillaries and/or other structures that may be at a stage where treatment may not be as successful. In addition, coupling imaging data with tissue integrity data may provide insight as to how well the vessels would be predicted to grow into the target tissue are (i.e., the skin region of interest) and mature into functional capillaries capable of providing nutrient exchange and waste removal.

Another embodiment may provide similar treatment for tissue regions that are already degenerative with components of this degeneration that may be due to hypoxia or ischemia and the resultant decrease in the necessary nutrients for matrix repair. For the relevant tissue(s) to “heal,” the necessary pathway for the nutrients required for aerobic energy metabolism could be restored. This might entail topical application of FGF-1 (either alone or in conjunction with other substances) and/or delivery of FGF-1 directly adjacent or into a hypoperfused vessel or tissue region. This treatment may be preoperatively planned with the proper imaging for mapping of the area to be treated. In addition, the FGF-1 (and/or other angiogenic factors or other necessary constituents) can be applied topically, injected, implanted and/or laid adjacent to various tissue regions using various delivery schemes, depending upon the pharmacologic properties of the various angiogenic factors and the consistency and fluid dynamics of their formulations. The treated tissue's healing environment may or may not be further enhanced with implants, dressings, prosthesis and/or other devices to protect and/or “unload” the tissue and/or vessel matrix if it is desired by the treating physician that a more optimal biomechanical environment could be achieved with this approach. The postoperative healing environment could be assessed with serial imaging studies and treatment could be modified if necessary. This modification could potentially alter the biomechanical properties of the tissue region, if desired. In addition, further treatment with the angiogenic factor could be performed depending upon the clinical and imaging information in the postoperative period.

In various embodiments, it may be desirous to identify a vascular condition that may reduce and/or negate the effectiveness of a given course of anticipated treatment in a given skin region. Various types of image data could be employed to perform such analysis, such as plain x-rays that could show severe hypoperfusion of major vessels, which might be a contraindication for localized angiogenic treatments of the vasculature and/or microvasculature adjacent and/or proximate to the skin wound. Image data may be used to detect a calcified and/or blocked vessel that could cause a vascular deficiency that eventually inhibits diffusive transfer in a given area of microcirculation. Where this obstruction (i.e., partial and/or complete) is located remotely from the given area of microcirculation, angiogenic treatments directly to the area of microcirculation may be relatively ineffective to significantly improve the patient's condition. Where multiple potential treatment areas may exist in a given vascular network supply to an area of interest, it may be advantageous to treat all of the multiple areas simultaneously and/or treat each area in a serial or “step-wise” fashion to desirably restore perfusion to the skin area of interest.

### Topical Application and Reduced Absorption

In various embodiments, an added benefit of topical therapy as a primary treatment modality can be a reduced opportunity for the FGF-1 to become absorbed into a patient's blood stream, as well as a significantly reduced opportunity for the FGF-1 to induce systemic and/or localized effects outside of the targeted skin region. For example, little or no absorption of topically-applied FGF-1 has been confirmed in animal studies, where no detectable FGF-1 was found in the bloodstream of animals after topical application. Moreover, in one exemplary dosing regimen involving human subjects, patients suffering from venous stasis ulcers and/or diabetic ulcers were treated with two topical doses of a compound containing approximately 0.3 or 3.0 μg/cm² in combination with heparin. Results from these individuals showed that topically-applied FGF-1 compounds were well tolerated and showed no drug-related adverse effects when applied to the wounds. In addition, pharmacokinetic analyses of serum samples from these subjects demonstrated that FGF-1 was at undetectable levels in the circulation after topical application (with a detection limit of the ELISA assay determined to be 30 pg of FGF-1-141).

In another exemplary dosing regimen involving human subjects, FGF-1 or a corresponding vehicle placebo was applied to normal skin, either as a single dose or as three doses applied over a period of five days. A second dosing regime was done using the same protocol, except treatment was applied to abraded skin. Additional dosing regimen were accomplished using the same protocol, but by applying multiple doses of FGF-1 or vehicle placebo into dermal punch biopsies, blister wounds and split-thickness wounds. In all of these regimes, no drug-related adverse events from the FGF-1 were observed. It should be noted that various combinations of one or more of these treatment approaches is contemplated in this invention.

One attraction of protein therapy can be that relatively small amounts of a very potent therapeutic agent can be topically applied and/or even injected into the ischemic area of interest to pharmacologically initiate the process of blood vessel growth and collateral artery formation. In addition, from pharmacokinetic data collected from human heart studies, it appears that once FGF-1 exits a tissue structure it can be largely cleared from circulation in less than 3 hours. This diminishes the risk of FGF-1 stimulating unwanted angiogenesis in other tissues of the bodies where it could potentially promote inappropriate angiogenesis and other adverse physiologic responses.

If desired, various delivery vehicles for FGF-1 could be employed in a topical formulation, such as those useful for transdermal delivery of materials to underlying skin layers, subcutaneous tissues and/or a patient's vascular system. For example, microcapsules and/or nanocapsules could be employed in a topical formulation that contain FGF-1, with the microcapsules and/or nanocapsules capable of transiting through the surface skin layers and delivering their FGF-1 payload into one or more subsurface environments. For example, see U.S. Pat. No. 5,993,831, the disclosure of which is incorporated herein by reference. If desired, the microcapsules and/or nanocapsules could be degradable and/or biodegradable, with the FGF-1 payload within such capsules optionally including a solid, semi-solid, liquid and/or biodegradable carrier that facilitates immediate exposure and treatment of subsurface tissues and/or that allows for timed-release of the FGF-1 payload to surrounding tissues.

Prosthesis to Protect and/or Offloading Damaged/Ischemic Tissue

In various embodiment, the diagnosis and treatments described herein can have particularly utility in combination with devices and/or instrumentation and/or procedures that “offload,” isolate, protect, limit the mobility of and/or otherwise provide temporary and/or permanent reduction in the localized loading of one or more ischemic tissue regions. A wide variety of such systems and/or procedures could be utilized in conjunction with the various treatments disclosed herein, which in various embodiments include offloading devices that concurrently include a dual capability of accepting an insert or replaceable “reservoir” of material for treating an external surface of the skin wound in a desired manner.

As previously described, it is believed that vascular insufficiencies leading to oxygen and nutritional insufficiencies in skin and related tissues are a significant contributor to the degradation, chronic non-healing and/or eventual “failure” of the relevant skin tissue structures. It is further known that continued direct pressure loading of a skin tissue wound can significantly reduce and/or obviate the wound's ability to heal, as well as incur intense pain to the patient. Such pressure loading can also further degrade the tissue structures. It is believed that the subsequent angiogenic treatment of the skin wound and/or underlying vascular insufficiency after such diagnosis could be facilitated by the use of one or more “wound offloading” systems, such that the skin wound is not subject to direct pressure for an amount of time sufficient for the skin wound to heal. This offloading, in conjunction with the increase in diffusive nutrition/waste removal resulting from the angiogenic treatment, has a significant opportunity to reduce, halt and/or reverse the effects of the earlier degradation.

The combination of angiogenic therapy with wound offloading devices desirably pharmacologically improves the nutrient exchange and waste removal of the skin tissues while unloading the tissues and supporting vasculature and/or microvasculature mechanically. This desirably optimizes the clinical approach, because the vasculature supplying the damaged skin can still be further damaged and/or compressed by pressure loading, while the skin tissue and/or healing wound itself can be further damaged by direct loading. Lessening the spot strain on vulnerable skin tissues can optimize the environment for healing, and the combined efforts to reduce loading and improve local blood flow by administration of FGF-1 or similar angiogenic compounds into and/or around the skin wound can stabilize and/or increase the effectiveness of the microvasculature as a nutrient exchange tissue.

In the case of wounds to load-bearing surfaces of the feet, many physicians feel that footwear as a means of healing open wounds is rarely desirable, but cost pressures promote treatment of such wounds in an outpatient setting. At least one study estimates that six weeks of treatment in an outpatient setting using a total contact cast (TCC) costs the same as a single day of inpatient treatment. Currently, TCC represents the gold standard for the treatment of forefoot and midfoot (Wagner grade 1-2) diabetic and neuropathic ulcerations; however, reduction of heel pressures with this device remains controversial. This type of specialized casting desirably protects the foot from trauma, immobilizing skin edges and reducing edema. It also seeks to decrease pressure over the ulcer by redistributing the weight bearing load over a greater plantar surface area. Molding the bottom of the cast to the bottom of the foot desirably causes the entire sole to participate in the force distribution, resulting in lower pressures, with an objective of reducing the peak pressure on the damaged region(s) of the foot.

While the TCC device is accepted as an effective, low-risk, and inexpensive treatment for plantar diabetic foot ulcers, it also has several disadvantages, including joint stiffness, muscle atrophy, the possibility of new ulcerations and skin breakdown, labor-intensive application, and possible laceration of the patient during cast removal. The cast cannot be removed by the patient, and thus it severely limits the patient's movement for the duration of casting and does not allow patients, family members, or health care providers to assess the foot or wound on a daily basis. Many treatment centers may not have a skilled health care professional or cast technician available with adequate training or experience in TCC, and improper cast application can irritate the skin, potentially leading to frank ulceration. In many cases, TCC makes sleeping and bathing difficult for patients, and certain casting designs may exacerbate postural instability. Total contact casting is also generally contraindicated in cases involving concomitant soft tissue infection, osteomyelitis, and/or ischemia, and may not be appropriate in the treatment of heel ulcers, due to the excessive pressure transmitted to the posterior foot. Contact dermatitis and fungal infection can often occur, which must be treated with appropriate topical medications and temporary removal and/or replacement of casting (if necessary). Although TCC is an ambulatory procedure, the patient is required to limit ambulation to one-third of normal. This often requires counseling and close follow-up while the cast is in place. Vascularity must be carefully evaluated before cast application, and it has been discovered that TCC causes postural instability in the ambulating patient as compared to a tennis shoe or removable cast walker; therefore, the well-being and safety of the patient must be strongly considered before recommending the device.

A wide variety of other prosthesic devices are available for use with wounds to load-bearing surfaces of the feet, and each device has attendant advantages and disadvantages, many similar to those described for TCC. Such additional devices can include various non-weight bearing devices (i.e., crutches, wheelchairs, walkers), standard below-knee casts, the Charcot Restraint Orthotic Walker (CROW), prefabricated walkers, the Integrated Prosthetic and Orthotic System (IPOS), the Orthowedge, the healing sandal, the Reverse IPOS heel relief shoe, the L'Nard splint/multiboot, the Ankle Foot Orthoses (AFO), the Patella tendon bearing brace (PTB), the prefabricated pneumatic walking brace (PPWB), the MABAL shoe/Scotch boot, and felt and foam total contact padding.

In various embodiments, the imaging and analysis of a skin wound and related anatomy can desirably be utilized to design and manufacture a prosthesis that can be worn by the patient to protect the skin wound while allowing a desired level of ambulation and concurrently treating the wound in a desired manner. In various embodiments related to skin wounds of the lower extremity and/or “load bearing” surfaces, the imaging and analysis of a skin wound and related anatomy can be utilized to design and manufacture a prosthesis that desirably “offloads” the skin wound for the specific patient, while concurrently treating the wound in a desired manner.

Various embodiments described herein include the use of computer aided design and/or computer aided modeling (CAD-CAM) systems to model, design and build a prosthesis for use in treating a skin wound. Desirably, prosthesis can be constructed using a rapid prototyping (“RPT”) process, Direct Digital Manufacturing (“DDM”), 3D Printing (i.e., Additive Manufacturing) or other process suitable for manufacturing unique individual units or other devices that would be manufactured either as a one-off or low volume item. Rapid prototyping is the automatic construction of physical objects using solid freeform fabrication. The first techniques for rapid prototyping became available in the late 1980s and were used to produce models and prototype parts. Today, they are used for a much wider range of applications and are even used to manufacture production quality parts in relatively small numbers. Some sculptors use the technology to produce complex shapes for fine arts exhibitions.

In various embodiments, a model of the patient's anatomy can be obtained from image data, which can include anatomical information of the patient's soft and bony structures of the affected extremity. The anatomical model can then be utilized to derive and/or create a prosthesis appropriate for the patient's anatomy, which could include the design of a unique prosthesis for the patient as well as the use of a pre-designed prosthesis, which may require manipulating and/or “fitting” of the pre-designed prosthesis to the specific patient anatomy. Desirably, the model will accommodate the underlying patient anatomy, and may also accommodate projecting and/or “pointy” subsurface bony features to desirably avoid further ulceration and/or skin damage while the prosthesis is being worn by the patient.

To accommodate the skin wound(s), one or more openings or depressions can be modeled in the prosthesis which desirably “offloads” the skin wound(s). Desirably, each opening will accommodate the entirety of a wound, as well as an offset or “margin region” surrounding the skin wound, which desirably ensures offloading of the wound and the minimization of any “edge effects” which may negatively affect the healing of the skin wound. Depending upon the location of the skin wound and the load bearing nature of the tissues, the shape and/or depth of the offset may vary, with virtually any shape opening being contemplated, including openings of circular, oval, symmetrical and/or non-symmetrical or any other geometric shape. If desired, the prosthesis body could be formed from a relatively rigid material such as plastic or metal, with a support and/or cushioning material such as closed-cell foam, silicone or rubber included on a skin-facing surface of the prosthesis. In such embodiments, the opening could be formed in the support and/or cushioning material, rather than in the prosthesis body, if desired.

Once the virtual 3D model (i.e., from the computer aided design (CAD) or animation modeling software) of the prosthesis has been created, it will desirably be transformed by a rapid prototyping machine into thin, virtual, horizontal cross-sections, with the machine creating each cross-section in physical space, one after the next until the model is finished. The virtual model and the physical model will desirably correspond almost identically, but may vary depending on the resolution used in the RPT process. With additive fabrication, the machine reads in data from a CAD drawing and lays down successive layers of liquid, powder, or sheet material, and in this way builds up the model from a series of cross sections. These layers, which correspond to the virtual cross section from the CAD model, are joined together or fused automatically to create the final shape. The primary advantage to additive fabrication is its ability to create almost any shape or geometric feature. A large number of competing technologies are available in the marketplace. As all are additive technologies, their main differences are found in the way layers are built to create parts. Some melt or soften material to produce layers, while others use layers of liquid materials that are cured. In the case of lamination systems, thin layers are cut to shape and joined together. Among the various RPT technologies are selective laser sintering (SLS), direct metal laser sintering (DMLS), fused deposition modeling (FDM), selective laser melting (SLM), stereolithography (SLA), laminated object manufacturing (LOM), electron beam melting (EBM), Laser Engineered Net Shaping (LENS), laser cladding, and 3D printing (3DP).

In various embodiments, the layering of the prosthesis may be particularized to optimize the strength and/or durability of the prosthesis. If desired, individual layers can be cross-weaved top maximize construct strength and/or reduce the potential for weakness or fracture along one or more intra-layer boundaries. In other embodiments, the layering may be particularized such that anticipated stresses loading intra-layer weaknesses can be minimized. For example, a prosthesis for a foot could be manufactured by layering the material from the medial side to the lateral side of the prosthesis, creating layer lines extending along an anterior to posterior axis that should be highly resistant to forces induced on the prosthesis by the patient's gait propulsion and “push off” of their foot.

Once the prosthesis has been created by the manufacturing machinery, it could be utilized immediately and/or might require additional post-processing steps such as the addition of one or more layers of support and/or cushioning material (as previously described). Desirably, the finished prosthesis can then be sent to the physician and/or patient for fitting and use during the treatment regimen.

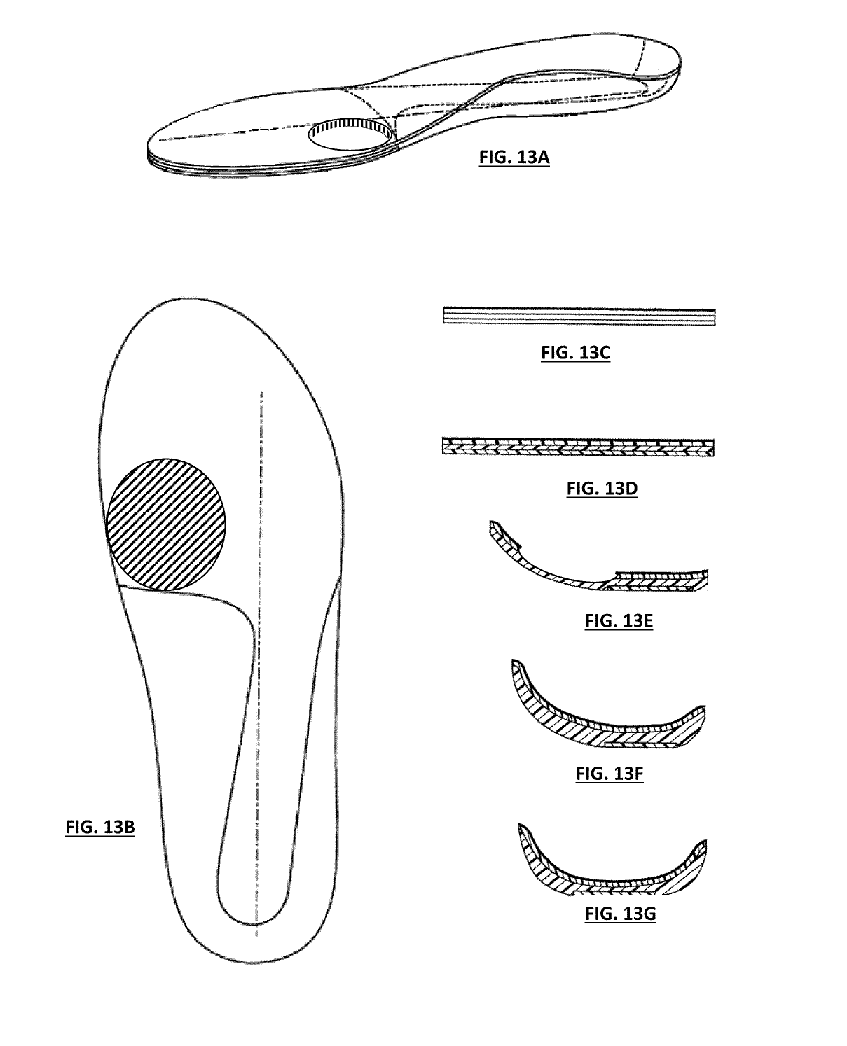

FIGS. 13A through 13G depict various views of one exemplary embodiment of a tissue offloading prosthesis, such as a customized sole support or orthotic that can be useful for treating foot ulcers. The prosthesis incorporates various features that will desirably facilitate use of the prosthesis during treatment of the foot ulcer using angiogenic factors. The prosthesis is desirably customizable to the shape and support requirements of the patient's foot, and in various embodiments the patient's foot can be imaged and/or measured, with the image data in various embodiments depicting both the contours and shape of the outer surfaces and sole of the foot, as well as image data reflecting the underlying soft tissues and/or hard tissues (i.e., bone) of the foot. Desirably, the image data can be used to model the foot, and potentially identify any hard or soft tissues that may be contributing directly to the foot ulcer or other skin wounds as well as tissues that may be indirectly contributing to vascular deficiencies by constricting and/or blocking vascular and/or microvascular flow with within the foot. Various embodiments can include obtaining perfusion data of the blood flow within the foot and/or extremity, and in some embodiments such data could be obtained from the patient's foot while in a weight-bearing condition (i.e., standing MRI, etc.), if possible. If desired, additional patient anatomy may be imaged, such as the patient's opposing extremity and/or connecting anatomy, to identify other anatomical abnormalities that might be addressed by proper modeling and design of the prosthesis (i.e., increasing the thickness of the prosthesis to address a gait abnormality).

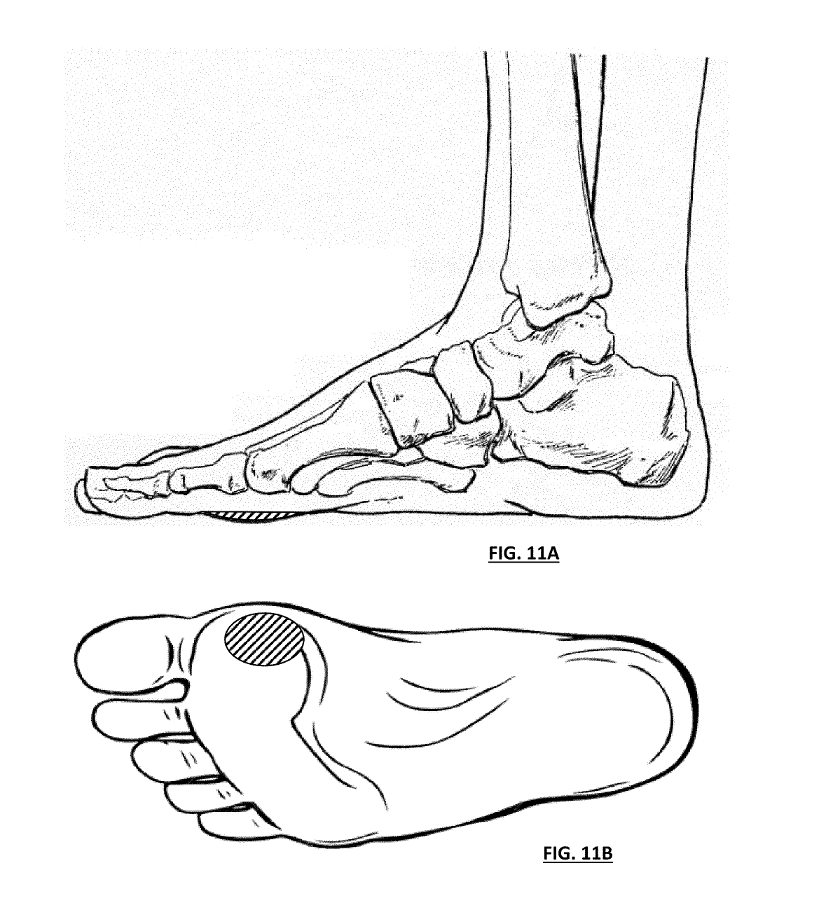

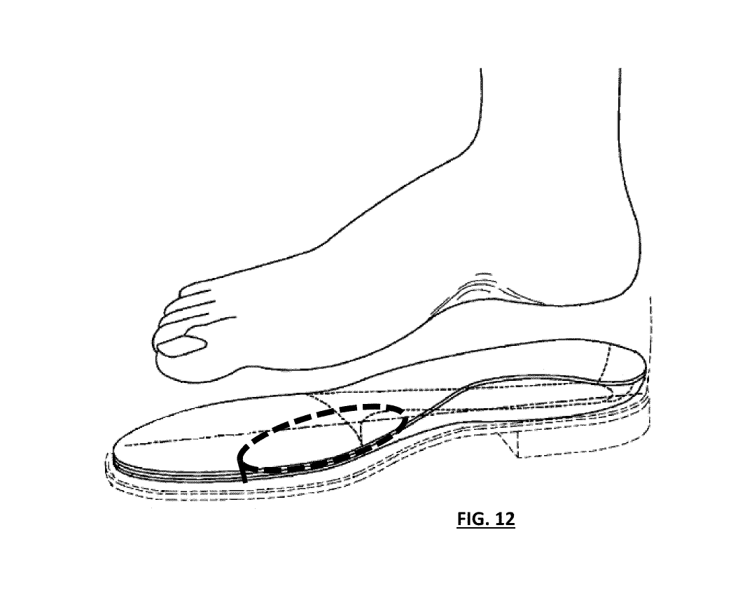

Once a model of the patient's foot anatomy has been obtained, an appropriate prosthesis can be designed that provides optimal support to the patient's foot while “offloading” the relevant sore(s) in a desired manner. In the embodiment of FIGS. 11A and 11B, a skin ulcer has formed on the medial pad of the patient's right foot, necessitating treatment with angiogenic factors. If desired, the patient's foot can be imaged, and a prosthesis model can be designed to incorporate a depression in the prosthesis proximate the skin ulcer (See FIG. 12). In this embodiment, the depression has been modeled and ultimately formed larger than the skin ulcer so as to offload some portion of the healthy tissue margin proximate to the ulcer, although in other embodiments the depression may be the same size and/or smaller than the ulcer, depending upon a variety of factors including ulcer size and support desired in various regions of the foot. If desired, the edges of the depression may be tapered, relieved and/or rounded to desirably alleviate any potential edge effects on the surrounding tissue and vasculature.

As best seen in FIG. 13E, which is a side view of the prosthesis and depression of FIG. 13B, the prosthesis body includes an underlying base material, with a surface padding formed from a firm yet pliable material such as closed cell foam, rubber, silicone or the like. In this embodiment, the padding material is absent from the depression, as well as some upper portion of the base material (although the base material desirably remains solid underneath the depression). Desirably, the depression will be formed such that, when the prosthesis is in contact with the patient's foot, the skin ulcer will be positioned within the depression.

In various additional embodiments, an insert or other device could be provided that is sized and/or configured to fit within one or more of the openings or depressions in the prosthesis, the insert desirably containing an angiogenic compound as described herein (optionally with other constituents, as described herein). In various embodiments, an insert could include a delivery or “deployment” feature which facilitates dispensing and/or application of the angiogenic compound and/or other constituents to the wound surface in a desired manner, such as through a permeable skin-facing wall of the insert. In various embodiments, the various body movements of the patient could desirably impel such delivery by simple compressive pressure on the insert, or a deployment device, pump or other arrangement could be provided to deliver the angiogenic compound as desired. In various alternative embodiments, the compressive pressure could be applied to peripheral portions of the insert by the healthy tissues at the margin of the ulcer, while the ulcer itself desirably experiences little or no substantial contact with the insert surface.

In various embodiments, the interior surface of the opening will desirably be recessed from the surrounding surface of the prosthesis, and if desired one or more edges of the opening may be tapered, curbed and/or otherwise transitioned to reduce and/or eliminate edges effects on the underlying tissues and/or vasculature. In other embodiments, the edges may be sharp or abrupt.

In various embodiments, the prosthesis may include a variety of surface features to accommodate the underlying patient anatomy, if desired. For example, the sole of the foot is one of the most vascularized regions in the human body. The subcutaneous tissue in the sole has adapted to deal with the high local compressive forces on the heel and the balls (distal end of metatarsals) of the big and little toes by developing a system of pressure chambers. Each chamber is composed of internal fibrofatty tissue covered by external collagen connective tissue. The septa (internal walls) in these chambers are permeated by numerous blood vessels. In various embodiments, it may be desirous to provide a “relief surface” in the skin-facing side of the prosthesis to accommodate a unique anatomical feature, such as a nerve complex or blood vessel, to desirably remove pressure on the specific structure. Similarly, it may be desirous to provide a relief for underlying bony protrusions or other anatomical features, which may include various anatomical features identified during an imaging scan as described herein

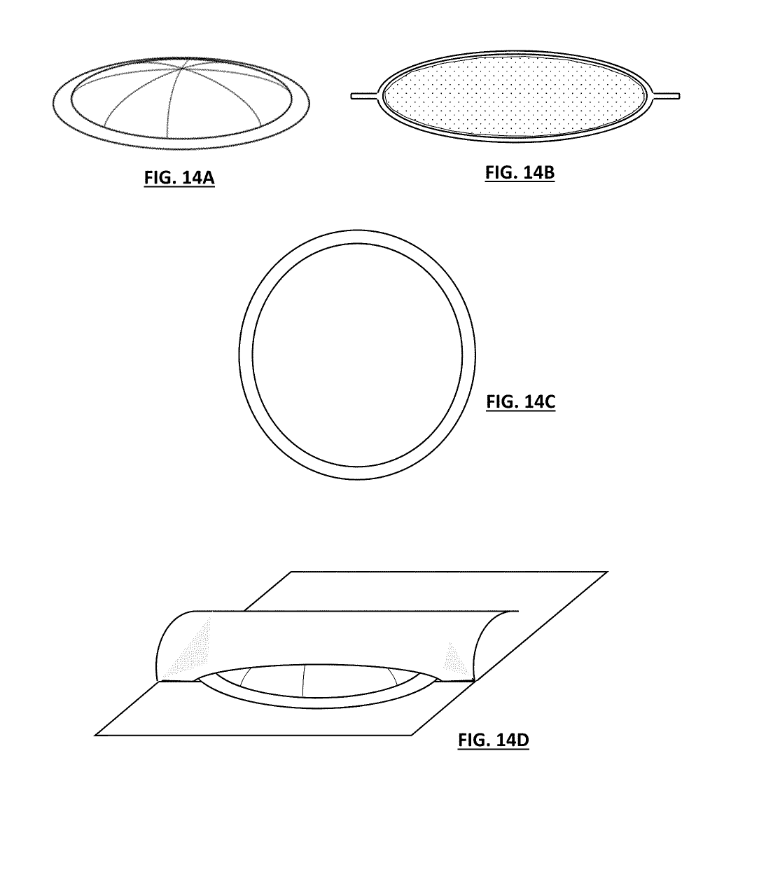

FIGS. 14A through 14C depicts various views of one embodiment of an insert or pad that can serve as a “reservoir” of FGF-1 and/or other medicaments for topical treatment of the skin ulcer. In this embodiment, the insert can include a central portion for containing the various medicaments (optionally including the FGF-1), with at least one outer skin-facing surface comprising a membrane that is permeable to the various medicaments. Desirably, a medicament contained within the central portion can pass through the permeable membrane and onto the surface of the insert, which can then transfer the medicament to the surface of the skin ulcer and/or surrounding tissue via direct contact. In various embodiments, a flexible porous, spongy or other medicament retaining material can be positioned within the central portion, with the various medicaments contained within and/or incorporated into the material. Desirably, the porous material can comprise a material “softer” and/or more pliable than the surrounding padding material, which could include being formed from a porous material having a modulus of elasticity less than a corresponding modulus of the padding material. If desired, the insert can be sized to fit within the depression, with a skin facing surface of the insert being positioned below the level of the surrounding padding, even with the level of the surrounding padding, or above the level of the surrounding padding. If desired, an adhesive, hook and loop fasteners, or other retaining arrangement could be provided on one or both of the insert and the corresponding depression surface to retain the insert in a desired location and/or position within the depression.

In various embodiments, the prosthesis and retained insert will desirably experience pressure or stress during the patient's activities of normal daily living (i.e., walking), with various patient actions resulting in compressing, squeezing or otherwise impelling the medicament retaining material to expel some portion of the medicament through the membrane and into contact with the wound or surrounding skin surface. Desirably, such expelling action can occur on an occasional and/or continuous basis during daily activities, with the added benefit of reapplication of medicament to the ulcer and surrounding tissues on a periodic basis without requiring direct patient interaction. Desirably, the insert can be removed from the prosthesis after a sufficient period of time, with a new insert substituted into the prosthesis for further use.

As previously noted, one significant limitation in the use of FGF-1 in treating skin ulcers and/or other anatomical damage is the limited “half-life” of FGF-1, in which the efficacy of FGF-1 decreases significantly once FGF-1 reaches an elevated temperature, which can include ambient room temperature and/or “body” temperature. However, where individual inserts can be refrigerated, frozen and/or otherwise cooled prior to use, the limited half-life of FGF-1 can be ameliorated and/or be of little or no concern. For example, a prosthesis incorporating replaceable medicament inserts such as those described herein can allow a patient to remove a used insert from the prosthesis, which typically has been at an elevated temperature for a period of time during use, and replace the used insert with a new insert just recently removed from chilled storage. Desirably, the new insert will contain non-degraded FGF-1, which will begin to degrade at a typical rate once it has been inserted and applied to the patient's wound. Once this new insert has been used for a desired period of time, it can also be discarded and replaced with an even newer insert again recently removed from chilled storage, with the process repeating for the duration of patient treatment. In various embodiments, a patient undergoing outpatient treatment of a foot ulcer can desirably be provided with a number of inserts that can be refrigerated and/or frozen in the patient's home and/or room refrigerator, with the inserts removed and replaced by the patient on a periodic basis.

In various embodiments, the insert might comprise a heat-absorbing or “cooling” gel that can potentially provide a cooling sensation and/or alleviate pain on the patient's skin wound for some period of time after initial use. If desired, the gel could release medicaments such as angiogenic factors as the gel increases in temperature during use.

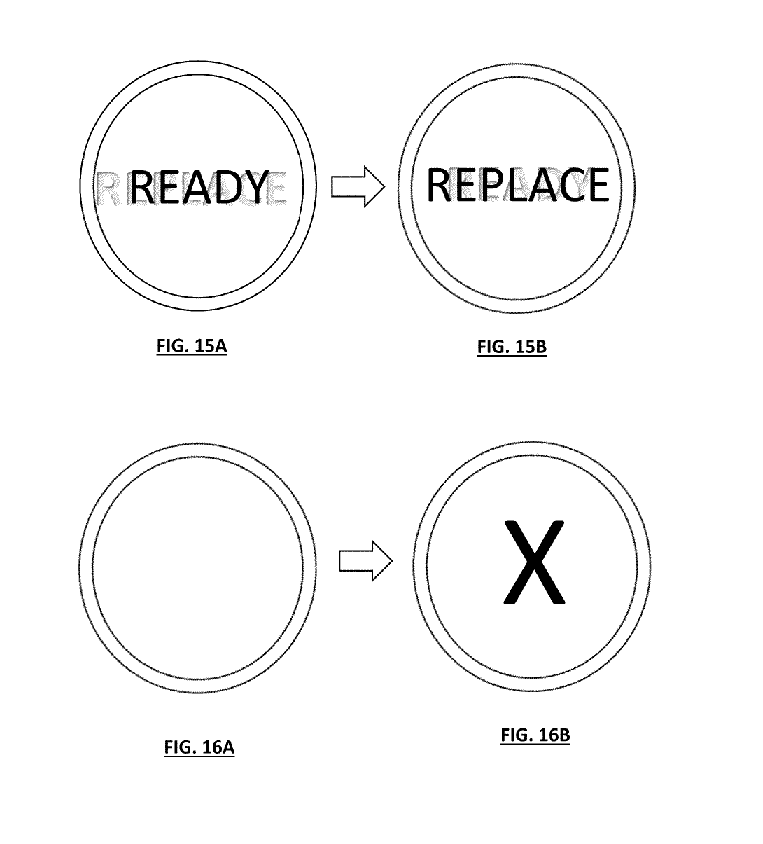

In various embodiments, an insert can incorporate an indicator or “tell-tale” that can be used to visually differentiate a used or degraded insert from an unused insert (See FIGS. 15A through 16B). For example, an insert could incorporate a thermochromic ink which provides a visual identification of when an insert has been in an uncooled state for a specified period of time. For example, a thermochromic time-temperature indicator can be incorporated into the skin-facing surface of an insert (i.e., by surface printing), with the ink particularized to change color once the insert temperature exceeds a set temperature (i.e., room temperature) for a given period of time (i.e., one or two days). Desirably the ink will provide a quick and convenient visual indication that it is time to change the insert, and the patient or caregiver can remove the insert from the prosthesis and replace it with a new insert just removed from cooled storage.

If desired, inserts could be individually packaged, such as by being enclosed in a “peel pouch” or similar packaging (see FIG. 14D). Alternatively, the inserts could be bulk packaged in a recloseable container, if desired.

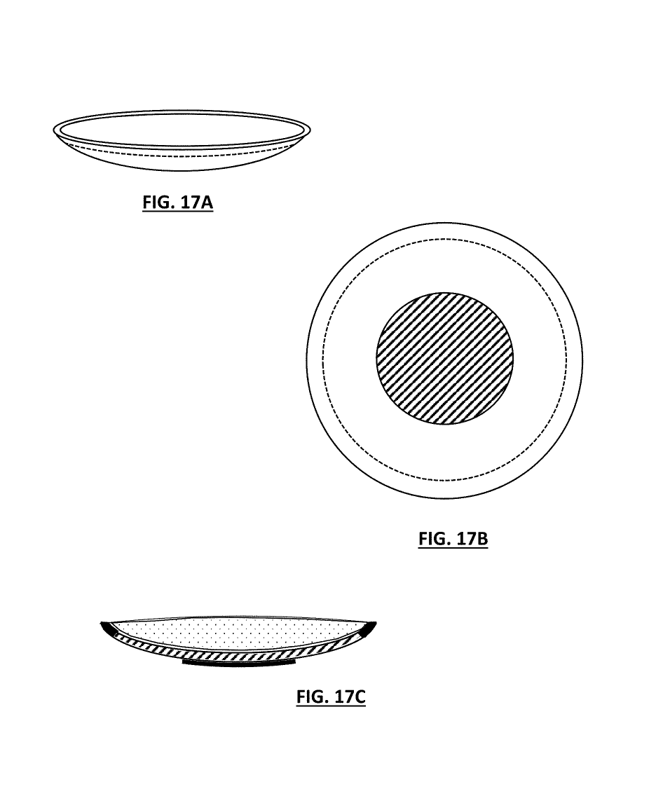

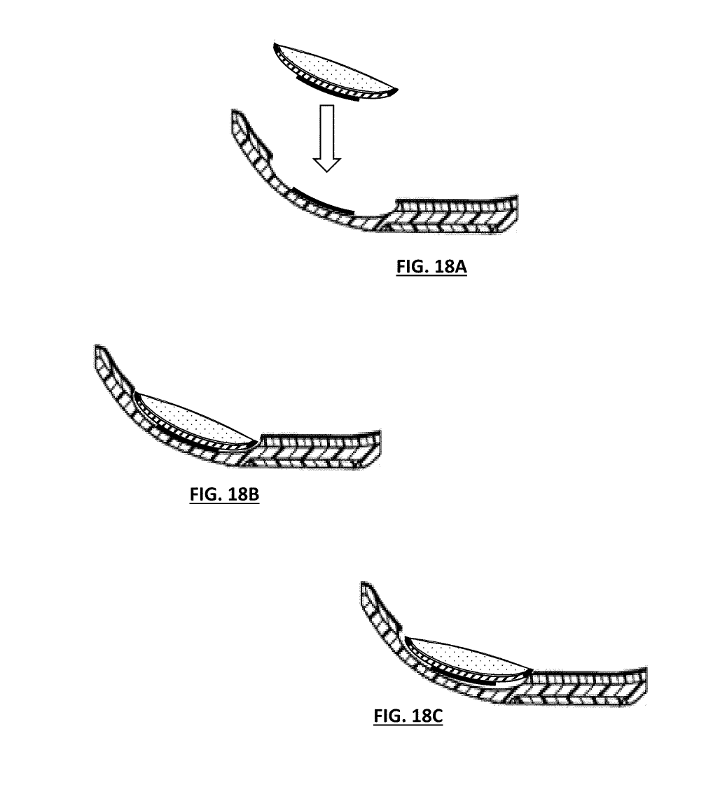

In various embodiments, the entire outer covering of the insert (and/or the entirety of the insert) might comprise a flexible material, with one or more outer surfaces of the insert comprising a medicament permeable layer. Alternatively, portions of the insert material could comprise non-permeable flexible materials, such as some or all of the “back” surface of the insert, wherein the front surface is intended to be in contact with the patient's skin (See FIGS. 17A through 17C). If desired, this back surface could alternatively comprise a hard, relatively inflexible material, if desired. In one exemplary embodiment best depicted in FIG. 17C, the peripheral edges of the insert might incorporate a flexible, relatively impermeable material, while the remainder of the back side can be relatively inflexible. Such a design could facilitate placement of the insert within a load-bearing prosthesis, such as described previously (see also FIGS. 18A and 18B), and desirably allow proper operation of the insert even some portion of the peripheral edge might extend above the padding surface (see FIG. 18C), such as where the insert may not be fully aligned within the depression.

In various embodiments, an insert could be used individually (i.e., without an associated prosthesis) by the patient or caregiver to apply medicament directly to a skin wound or ulcer. In such a case, the insert could be used in a manner similar to antiseptic wipes, with a single insert used to treat multiple wounds, if desired, and then discarded after such use. In another alternative embodiment, the insert could be used to topically apply medicament to one or more skin wounds (i.e., wiped over the skin wounds), and then the insert could be placed into a prosthesis to provide longer-term treatment for other ulcers, if desired. In this manner a single insert could be useful in treating multiple ulcers and/or could be used to treat skin areas in danger of developing ulcers, along with the primary ulcer treatment using the combination prosthesis and insert, as described previously.

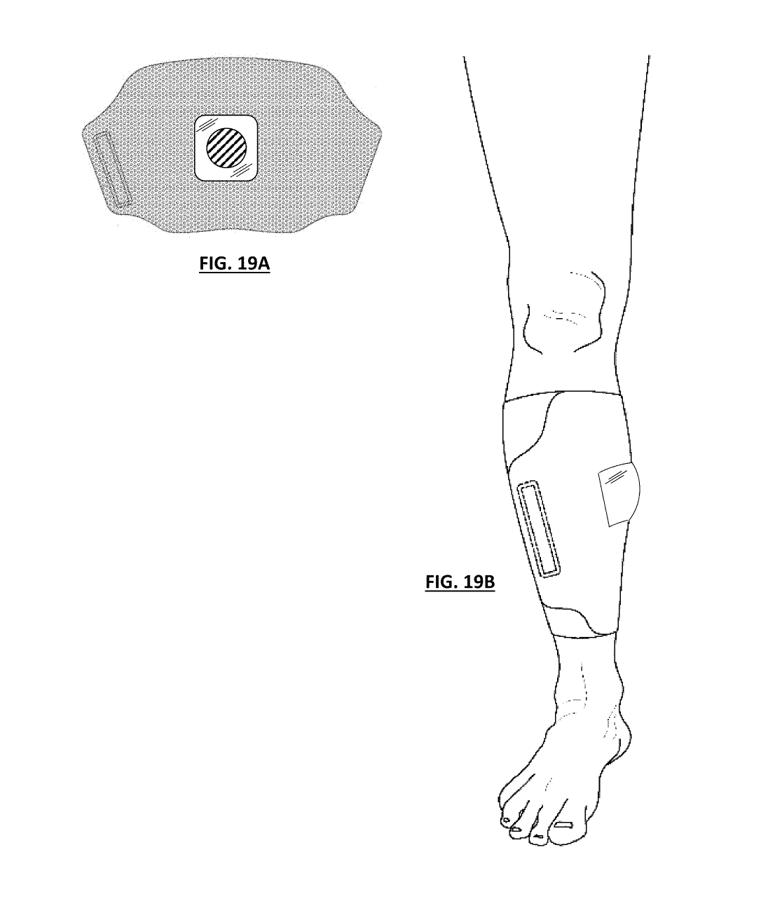

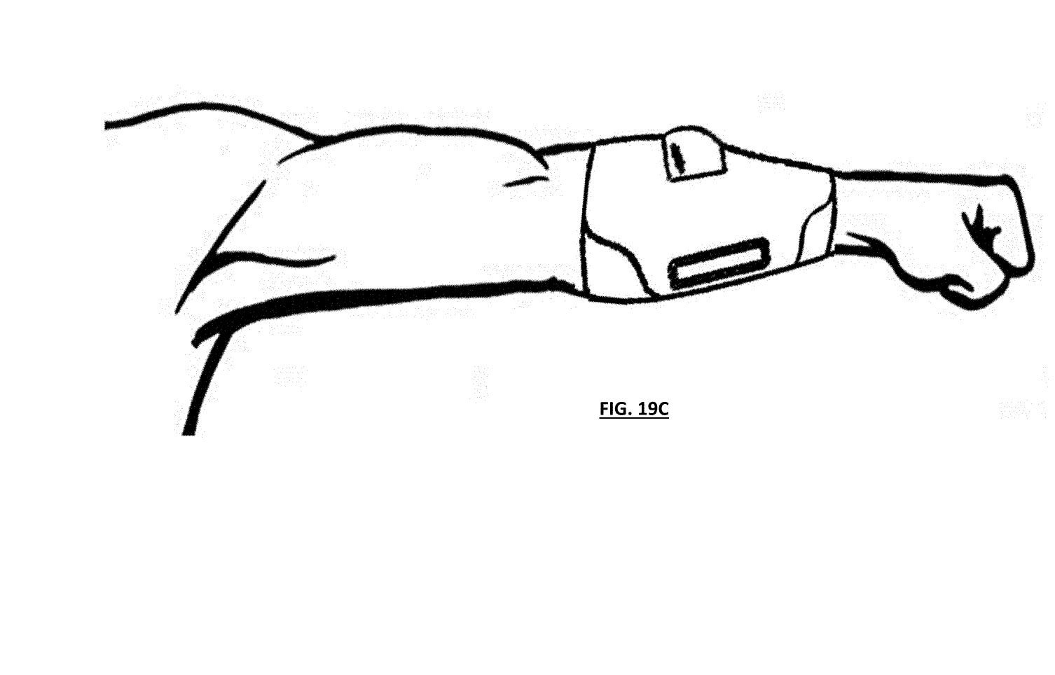

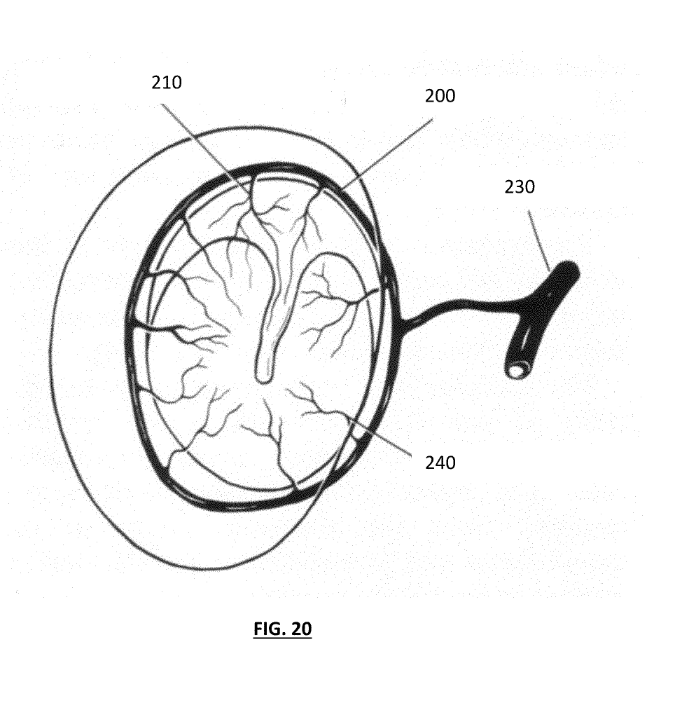

FIG. 19A depicts an alternative embodiment of a prosthesis for use in treating skin ulcers and other wounds with angiogenic medicaments. This embodiment comprises a compression-type bandage or wrap, with a pouch or pocket for accommodating an angiogenic medicament insert. Desirably, the pouch will include a clear or transparent portion, such as a central portion of the pouch and/or a pouch periphery region, which desirably allows the patient or a caregiver to view some portion of the skin surface underlying the pouch. In addition, the inner surface of the pouch will desirably allow medicament from the insert to pass through and/or around the pouch material and contact the underlying skin wound and/or adjacent tissue. Desirably, once an insert is placed into the pouch, the prosthesis can be positioned over the skin wound requiring treatment, with the clear portion allowing visual verification that the insert is properly positioned over the wound. Alternatively, the prosthesis could first be positioned over the skin wound requiring treatment, with the clear portion allowing visual verification that the skin wound is in a desired position relative to the pouch region, and then the insert can be placed into the pouch. Various embodiments for use with extremities could include a leg prosthesis (FIG. 19B) and/or an arm prosthesis (FIG. 19C).

In various embodiments, patient movement and/or patient actions can desirably result in compressing, squeezing or otherwise impelling the medicament retaining material within the insert to expel some portion of the medicament through the permeable insert membrane and into contact with the wound or surrounding skin surface. Alternatively, the patient could apply external direct pressure to the pouch for a short time on a periodic basis (i.e., by pressing their opposing hand down on the outer surface of the pouch), which would desirably re-apply the angiogenic medicament to the surface of the skin wound.

In another alternative embodiment, a prosthesis for use in treating skin could comprise an adhesive bandage or pad, with a pouch or pocket formed therein. The pad could be adhered to the skin of the patient, if desired, with an insert contained within the pouch at a location adjacent to the skin wound or ulcer. As previously described, the patient's movement and/or outside forces could impel the insert to extrude, exude and/or otherwise deliver a medicament to the surface of the skin wound and optionally to adjacent healthy skin tissue. Desirably, some portion of the pouch will be transparent, allowing the patient or a caregiver to view the skin wound or ulcer through the transparent portion to facilitate wound assessment and/or proper positioning of the insert.

In various embodiments the pouch could include a closeable opening on either or both of the back side and/or skin facing side of the prosthesis. The ability to remove and replace the insert without removing the prosthesis from the treated anatomy may be particularly useful in certain situations, such as where removal and replacement of the prosthesis would be difficult for the patient to accomplish unassisted (i.e., where the prosthesis is on an arm, or in a location not directly reachable and/or viewable by the patient). In such cases, the removal and replacement of the insert from the exterior of the prosthesis can allow the patient to easily self-administer a new dose of angiogenic factor in an outpatient setting.

### Compositions

The various treatments and compositions described herein can comprise a wide variety of materials, including scaffolding materials that incorporate collagen, PLA, and/or fibrin. Fibrin incorporation has an added benefit of bonding readily to FGF-1, consequently significantly increasing the thermal tolerance and “half-life” of FGF-1. For example, where “wild type” FGF-1 has a half-life of approximately 15 minutes at 37 degrees C., heparin bound FGF-1 has a thermal stability to approximately 60 degrees C. and a mitogenic half-life at 37 degrees C. of 24 hours. The longer half-life significantly increases the opportunity for FGF-1 to be utilized in conjunction with a therapeutic treatment. However, even a 1-day half-life could lead to a nearly complete loss of activity during long duration treatments, depending upon the dosing regimen.

In various alternative embodiments, a composition comprising human recombinant fibroblast growth factor-1 (FGF-1₁₄₁) may be provided in sterile dropper bottles and/or incorporated into inserts (as previously described), with the composition cooled and/or refrigerated just prior to use. One exemplary dosage of the composition could comprise 180 μg/ml (˜3.0 μg FGF-1 per cm² wound area), administered topically three (3) times per week for up to 20 weeks and/or until complete closure of the wound or ulcer. If desired, a continued monitoring of the dermal ulcer can continue for 12 weeks post-treatment.

Fibrin matrices can additionally function quite usefully as adhesives and/or “thickeners” in angiogenic compositions, desirably facilitating placement and/maintenance of FGF-1 at a desired location of a targeted anatomy. Fibrin can “set up” in situ (in place), filling voids and irregular shapes if desired. Another advantage is that the growth factor can be incorporated at the time of polymerization, which can serve to distribute the FGF-1 throughout the fibrin in a uniform and/or a non-uniform distribution, as desired. The ability to tie the drug delivery and degradation to cellular infiltration can be utilized to tailor the composition delivery to the individual patient's healing rate. Moreover, aside from improving the biological half-life of FGF-1, the binding of the FGF-1 receptor sites to fibronectin can protect the FGF-1 within the fibrin matrix, yet allow for sustained drug delivery from the matrix via leaching, polymer degradation and/or other means.

If desired, an angiogenic composition could comprise a graft material incorporating FGF-1 and a fibrin matrix, with the fibrin matrix, due to its own biological activity, serving as a basic scaffolding material for skin wound repair. In one exemplary embodiment, the fibrin could comprise a non-porous or porous matrix (i.e., 12% porosity and 100-200 mm pores). For a porous implant, the levels of porosity, the concentration of the growth factor, and/or the concentration of the fibrin matrix (which can affect the drug delivery rate and/or degradation rate) could be optimized for a particular size and/or shape of wound and/or anatomical location. Desirably, the graft material could induce complete epidermal regeneration with dermal filling of the full thickness defect, and minimal contraction (i.e., less than 20%). If desired, a pre-molded and/or moldable wound dressing comprising fibrin and/or other constituents could be utilized for treatment of skin ulcers. Alternatively, a moldable and/or alterable wound dressing comprising fibrin could be formed in-situ, with adhesiveness, polymerization, and/or flexural properties of the fibrin matrix being particularized for the wound topography.

In various additional embodiments, variations of FGF-1s can be used in which one or more amino acid insertions, deletions or substitutions are introduced by standard genetic engineering techniques, such as site-directed, deletion, and insertion mutagenesis. As previously described, the wild type FGF-1 three-dimensional conformation is known to be marginally stable with denaturation occurring either at or near physiologic temperature. FGF-1 binding to heparin increases the thermal inactivation temperature by approximately 20 C. Therefore, FGF-1 is typically formulated with therapeutically approved USP heparin. However, heparin is an anti-coagulant that can promote bleeding as a function of increasing concentration. In addition, some individuals have been immunologically sensitized to heparin by previous therapeutic exposure, which can lead to heparin-induced thrombocytopenia and thrombotic events. Mutations that extend the storage stability in vitro and biologic activity in vivo would allow FGF-1 to be formulated and dosed in the absence of exogenous heparin. These include mutations that decrease the rate of oxidative inactivation, such as replacement of one or more of the three cysteine residues by either serine or other compatible residues. In particular, as has been described by others, substitution of cysteine 117 by serine is known to substantially increase the half-life of human FGF-1 by decreasing the rate of oxidative inaction. Other mutations have been described that increase conformational stability by making amino acid changes in internal buried and/or external exposed amino acid residues. In the case of repeat dosing regimens, FGF-1s exhibiting greater stability and life-time might effectively decrease the frequency and number of repeated doses needed to achieve sustained exposure and greater efficacy. These stabilized mutants could allow longer duration dosing from slow release polymeric matrices and delivery systems.

In some embodiments a carrier solution or containing/metering device may be desired. Appropriate carrier solutions may be selected based on properties such as viscosity, ease of administration, ability to bind solution over a period of time, and general affinity for the agent delivered. Said solutions may be modified or additives incorporated for modification of biological properties. Starting solutions may include certain delivery polymers known to one who is skilled in the art. These could be selected from, for example: polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acryl substituted cellulose acetates, non-degradable polyurethanes, polystyrenes, polyvinyl fluoride, polyvinyl imidazole, chlorosulphonated polyolefin, and polyvinyl alcohol.

Depending upon the route of administration, non-invasive and/or minimally invasive imaging techniques may be desired in conjunction with a desired mode of treatment. Where subsurface administration is desired, such administration may be performed under fluoroscopy or by other means in order to allow for localization in proximity of the cause of hypoperfusion. Acceptable carriers, excipients, or stabilizers are also contemplated within the current invention; said carriers, excipients and stabilizers being relatively nontoxic to recipients at the dosages and concentrations employed, and may include buffers such as phosphate, citrate, and other organic acids; antioxidants including ascorbic acid, n-acetylcysteine, alpha tocopherol, and methionine; preservatives such as hexamethonium chloride; octadecyldimethylbenzyl ammonium chloride; benzalkonium chloride; phenol, benzyl alcohol, or butyl; alkyl parabens such as methyl or propyl paraben; catechol; resorcinol; cyclohexinol; 3-pentanol; and mecresol; low molecular weight polypeptides; proteins, such as gelatin, or non-specific immunoglobulins; amino acids such as glycine, glutamine, asparagine, histidine, arginine, or lysine; monosaccharides, disaccharides, and other carbohydrates including glucose, mannose, or dextrins; chelating agents such as EDTA (ethylenediaminetetraacetic acid); sugars such as sucrose, mannitol, trehalose, or sorbitol; salt-forming counter-ions such as sodium. For heparin-binding proteins, including FGFs, heparin may be incorporated into the formulation, which can bind and stabilize the protein against inactivation and degradation.

In various embodiments, treatment of hypoxic and/or ischemic tissue disease could include the use of a biocompatible, biodegradable and/or disposable implant. Said biodegradable implants can contain a biodegradable delivery system, or carrier, as well as angiogenic factors; said angiogenic factors could be capable of stimulating sufficient neovascularization to overcome local hypoxia. One preferred angiogenic factor is fibroblast growth factor 1 (FGF-1). However, other recombinant naturally derived, in vitro derived, and in vivo derived angiogenic factors may also be used. In some embodiments, the biodegradable implant which contains said angiogenic factors contains a carrier. The carrier is preferably chosen so as to remain at and/or within the implanted site for a prolonged period and slowly release the angiogenic factors contained therein to the surrounding environment. This mode of delivery allows said angiogenic factors to remain in therapeutically effective amounts within the site for a prolonged period. By providing said angiogenic factors within a carrier, the advantage of releasing said angiogenic factors directly into the target area is realized. In some embodiments, the implant's carrier is provided for topical application, while in others in may be provided in an injectable form. Injectability allows the carrier to be delivered in a minimally invasive and preferably percutaneous method. In some embodiments, the injectable carrier is a gel. In others, the injectable carrier comprises hyaluronic acid (HA).

In some embodiments, the carrier of the graft may comprise a porous matrix having an average pore size of at least 25 micrometers. Preferably, the porous matrix has an average pore size of between 25 micrometers and 110 micrometers. When the average pore size is in this range, it is believed that the porous matrix will also act as a scaffold for in-migrating cells capable of becoming cells stimulatory of angiogenesis in the targeted area. Numerous examples of organic materials that can be used to form the porous matrix are known to one of skill in the art; these include, but are not limited to, collagen, polyamino acids, or gelatin.

Said collagen source may be artificial (i.e., recombinant), or autologous, or allogenic, or xenogeneic relative to the mammal receiving the implant. Said collagen may also be in the form of an atelopeptide or telopeptide collagen. Additionally, collagens from sources associated with high levels of angiogenesis, such as placentally derived collagen, may be used. Examples of synthetic polymers that can be used to form the matrix include, but are not limited to, polylactic acids, polyglycolic acids, or combinations of polylactic/polyglycolic acids. Resorbable polymers, as well as non-resorbable polymers, may constitute the matrix material. One of skill in the art will appreciate that the terms porous or semi-porous refer to the varying density of the pores in the matrix.

Scaffold structures may be used in some embodiments for filling defects and/or anchoring or substantially causing adhesion between said implant and anatomical structures—such anatomical structures may include tissue and/or skin surfaces as well as bone, cartilage, nerve, tendon, ligament, other anatomical structures and/or various combinations thereof In some embodiments, the method of adhering said implant to said anatomical structures may be a gel. Said gel, together with said implant, could be placed inside an insert or can be applied and/or injected to the graft site, in some embodiments under arthroscopic fluid conditions. The gel can be a biological or synthetic gel formed from a bioresorbable or bioabsorbable material that has the ability to resorb in a timely fashion in the body environment.

Suitable scaffold agents are also known to one of skill in the art and may include hyaluronic acid, collagen gel, alginate gel, gelatin-resorcin-formalin adhesive, mussel-based adhesive, dihydroxyphenylalanine-based adhesive, chitosan, transglutaminase, poly(amino acid)-based adhesive, cellulose-based adhesive, polysaccharide-based adhesive, synthetic acrylate-based adhesives, platelet rich plasma (PRP) gel, platelet poor plasma (PPP) gel, clot of PRP, clot of PPP, Matrigel®, Monostearoyl Glycerol co-Succinate. (MGSA), Monostearoyl Glycerol co-Succinate/polyethylene glycol (MGSA/PEG) copolymers, laminin, elastin, proteoglycans, poly(N-isopropylacrylamide), poly(oxyalkylene), a copolymer of poly(ethylene oxide)-poly(propylene oxide), polyvinyl alcohol and combinations thereof.

In some embodiments, a pliable scaffold could be preferred so as to allow the scaffold to adjust to the dimensions of the target site of implantation. For instance, the scaffold could comprise a gel-like material or an adhesive material, as well as a foam or mesh structure. In one preferred embodiment, said scaffold could include a biodegradable, bioresorbable and/or bioabsorbable material. Said scaffold can be formed from a polymeric foam component having pores with an open cell pore structure. The pore size can vary, but in one preferred embodiment the pores could be sized to allow tissue or angiogenic ingrowth, while in other embodiments the pores could be optimized to contain the angiogenic agent and any other desired medicaments. In some embodiments, said pore size is in the range of about 40 to 900 micrometers. Said polymeric foam component can, optionally, contain a reinforcing component, such as, for example, woven, knitted, warped knitted (i.e., lace-like), non-woven, and braided structures. In some embodiments where the polymeric foam component contains a reinforcing component, the foam component can be integrated with the reinforcing component such that the pores of the foam component might penetrate the mesh of the reinforcing component and/or interlock with the reinforcing component, if desired. In some embodiments, said angiogenic growth factors could be predominantly released from a sustained delivery device by its diffusion through the sustained delivery device (preferably, through a polymer). In others, said angiogenic factors could be predominantly released from the sustained delivery device by the biodegradation of the sustained delivery device (preferably, biodegradation of a polymer). In some other embodiments, said angiogenic growth factors could be extruded through pores in one or more surfaces of the sustained delivery device by external compression of the device. In some embodiments, said implant comprises a bioresorbable material whose gradual erosion causes the gradual release of said angiogenic factors. In some embodiments, said implant comprises a bioresorbable polymer. Preferably, said bioresorbable polymer has a half-life of at least one month. Accordingly, in some embodiments, said implant comprises the co-polymer poly-DL-lactide-co-glycolide (PLG) admixed with said angiogenic factors.

In some embodiments, the implant could be comprised essentially of a hydrogel. Hydrogels can also be used to deliver said angiogenic factors in a time-release manner to the area of hypoperfusion. A “hydrogel”, as defined herein, is a substance formed when an organic polymer (natural or synthetic) is set or solidified to create a three-dimensional open-lattice structure that entraps molecules of water or other solution to form a gel. Said solidification can occur, e.g., by aggregation, coagulation, hydrophobic interactions, or cross-linking. The hydrogels described herein could rapidly solidify to keep said angiogenic factors in proximity to a skin wound and/or the blood vessel causative of hypoperfusion and/or the area associated with hypoperfusion. In some embodiments, said hydrogel could be a fine, powdery synthetic hydrogel. Suitable hydrogels would desirably exhibit an optimal combination of such properties as compatibility with the matrix polymer of choice, and biocompatibility. The hydrogel can include one or more of the following: polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, polyvinyl acetate, and sulfonated polymers.

In one alternative embodiment, a localized medical device and/or composition could be applied to a wound surface, to tissue adjacent to a wound surface and/or implanted using an attachment mechanism onto an anatomical structure that resides at a location adjacent to and/or remote from the area of hypoperfusion, such as adjacent to an external skin surface and/or within and/or proximal to a blood vessel supplying the area of hypoperfusion (i.e., for example, the peripheral vessels that feed to the microvasculature supplying the skin tissue). In various embodiments, attachment could be performed using an anchoring device; such as employing an anchoring device attaching a medical device to a soft or hard tissue proximal to an artery or vein. Said medical device could include an ability to provide time-course release of an angiogenic factor. Said medical device may include a solid or partially-solid casing with an internal gel-like fluid containing the desired angiogenic factor. Said gel-like fluid may be a cryoprecipitate, an administration matrix, or a composition of various polymers suitable for the sustained release of said angiogenesis promoting factor.

In one alternative embodiment, the medical device that adheres or attaches to the proximity of the hypoperfused area for the purpose of delivering the desired angiogenic factor could be placed near or in the proximity of the hypoperfused skin tissues. This medical device could be a reservoir for the formulation of the active delivered drug that is delivered over time to the wound and/or tissue surface. This device could be made of synthetic or biologic material and be able to be attached with adhesives, anchors or have positional stability without anchors.

### Tissue Grafts

In various embodiments, it may be desirable to utilize one or more tissue grafts to treat skin wounds, preferably in conjunction with the various systems, techniques and methods described herein, including various angiogenic treatments. For example, preparation for a tissue grafting procedure might desirably include the induction of angiogenesis and/or other treatments prior to, during and/or after the graft is implanted, with various treatment regimens being performed on the tissue graft, on the wound bed and/or on various combinations thereof. Poor circulation is well known to be a chief factor for tissue graft failure and lack of maturation. Treating the grafted area (i.e., the “wound bed”) before (or during) the tissue grafting procedure could potentially provide needed vascularity (and therefore, much needed oxygen and/or nutrients for tissue repair, adhesion and maturation). This treatment could be in the manner of introduction (i.e., topical application and/or injection) of FGF-1 alone or in a compound or vehicle such as xenograft, allograft, collagen matrix, synthetic, or other scaffolding. In one embodiment, the wound bed could be treated preoperatively to induce angiogenic growth into the relevant tissues, and then some portion of the surface of the wound bed could be resected prior to graft implantation, if desired, to expose the surfaces of the tissue graft to some portion of the newly developed vasculature. In another embodiment, the wound bed could be treated via injection, the tissue graft could be treated via injection, and/or the interface between the wound bed and the tissue graft could be treated with a topical compound.

In various embodiments, an extended, slow release dosing regimen could be employed, to desirably allow continuous delivery of a small molecule or protein, thereby avoiding the concentration peaks and troughs of intermittent oral or bolus injectable doses. This can be achieved using a pump or either an injected, topically applied and/or implanted polymeric gel or insert. If desired, biodegradable matrices could be used, including but are not limited to those containing one or more of the following: heparin, collagen, gelatin, fibrin, and alginates.

In a similar manner, the various treatments described herein can be used to prepare other tissues that are treated with tissue transplants and also have a high metabolic demand in the face of poor nutrient delivery. One example could be in the treatment of soft tissue loss in open fractures such as the tibia. It is well known that tibial non-unions have a poor blood supply and a tissue transfer, transplant, cell therapy, growth factor or other signaling molecules included in the tissue grafting could create a greater metabolic demand (both nutritionally and potentially waste-related), thus requiring greater nutrient delivery and/or waste removal. Combination therapy, including various aspects of the previously-discussed tissue grafting procedures with angiogenic treatment could be ascertained with the proper imaging studies and the type of angiogenic therapy, dose, distribution, delivery, and vehicle thoughtfully planned. This type of treatment could be useful in other similar ischemic tissue challenges, or other areas that have tissue defects in need of restoration throughout the body. This could include facial injuries or tumor or other musculoskeletal tissue defects. In various embodiments, the collection and analysis of imaging data and subsequent angiogenic treatments could be applied to virtually any anatomical area having one or more deficiencies and/or conditions that result in a large soft and/or hard tissue defect (i.e., due to trauma, tumor or some other disease) that may require a combined surgical reconstruction and angiogenic approach. For instance, an open tibia fracture with a poorly vascularized wound could be treated with various approaches described herein, including utilizing imaging data to plan a proper skin closure procedure using reconstructive surgical techniques along with angiogenic treatment. The angiogenic factors could be provided alone or in combination with a scaffold with or without stem cells.

### Stem Cells and Gene Therapies

In various embodiments, angiogenic treatments can be used in conjunction with other treatments, such as introduction and/or injection of stem cells, which may be embryonic stem cells or adult stem cells. Such angiogenic treatments could be used to prepare tissues for subsequent injection of stem cells, or angiogenic compounds could be injected concurrently with and/or after introduction of such cells. With regards to skin tissues or other tissues, growth factors, synthetic or treated allograft or xenograft tissue for scaffold (or extra-cellular matrix) and stem cells (each of which could be used separately or in varying levels of in combination with each other) could be utilized to “engineer” or otherwise modify skin tissue with the goal of regenerating living tissue. If the wound bed to be treated required that ischemia or hypoxia related causes needed to be diagnosed and treated first or in combination with the tissue engineering techniques (or if such treatment could be optimized if such approaches were employed), then the diagnosis and treatment could be for ischemic skin conditions or other pathologies such as described herein.

In addition, it may be determined that a combination of stem cells, engineered tissue, scaffold and/or growth factors (or various combinations thereof) could be enhanced by combining angiogenic factors such as FGF-1 in its native state or through an FGF-1 mutant (i.e., through protein engineering technology) or any other appropriate angiogenic factor. In this embodiment, the regenerative implant would desirably be selected and/or designed to not over-utilize the nutrients available in the wound bed. A limiting factor of regenerative therapy may be nutrient availability, oxygen supply, diffusive transport limitations and/or waste disposal constraints on any therapy that seeks to increase the local anatomical cellular population and metabolic rate. In combination therapy, nutrient delivery to the affected tissues may be desirably enhanced through increasing the population and/or density of the dermal and/or sub-dermal microvasculature.

Combination therapy could also include tissue engineered skin material that is transplanted into a wound bed made available by removing some or all previous degenerative wound material and/or healthy tissues. To provide nutrients for this transplant, angiogenic therapy, with or without concurrent skin grafting and/or tissue reconstruction, if needed, could be included. In addition, this combination therapy could be further enhanced with growth factors or other signaling molecules and embryonic or adult stem cells and various types of scaffold. The preoperative planning could desirably map the areas to be treated. Preoperative imaging, modeling and/or assessment, as described before, could be used to analyze the metabolic demands of the combination transplant and the state of the nutrient pathway that is required to support the transplant. Detailed preoperative planning, using imaging modalities already discussed (or imaging modalities not yet invented or used for this type of procedure) of the nutrient demands of the transplant and the subsequent translation of this imaging data into the proper amount, delivery, vehicle, approach, whether existing tissues should be altered and/or perforated, thinned or otherwise reconstructed to improve diffusion, what other anatomical areas might require treatment and how that information impacts the treatment plan and other yet unknown factors could all be information utilized when planning the regenerative therapy.

A similar approach could be used in connection with other joint structures and/or other tissues and organs, including structures such as the heart. One main dysfunction associated with ischemic heart disease appears to be a loss of perfusion of oxygenated blood to the heart tissue. If stem cell, gene therapy, protein therapy, tissue therapy or any combination thereof were implanted within heart tissue and/or otherwise directed towards the tissue of the heart, the metabolic demands of that transplant could be calculated with preoperative imaging and the proper angiogenic treatment delivered based upon that calculation. Alternatively, if the imaging demonstrated a range of breakdown of the delivery pathway to the transplanted tissue, cells, proteins, genes or any combination thereof, then a more non-specific dose of angiogenic therapy might be desired. The angiogenic treatment could be initiated, based on imaging data, prior to the regenerative treatment so that angiogenesis would already be present when the transplant is performed. In addition, the angiogenic treatment could be combined with the tissue/cell/signal transplant (or other regenerative embodiment), providing capillary growth and nutrient delivery to enhance healing of the transplant at the time of the procedure or subsequently after surgery. Administration of such factors could be accomplished prior to, during and/or after such surgery to the patient and/or the tissue transplant, as desired.

In various alternative embodiments, genes can be introduced from exogenous sources so as to promote angiogenesis. It is known in the art that genes may be introduced by a wide range of approaches including adenoviral, adeno-associated, retroviral, alpha-viral, lentiviral, Kunjin virus, or HSV vectors, liposomal, nano-particle mediated as well as electroporation and Sleeping Beauty transposons. Genes with angiogenic stimulatory function that may be transfected include, but are not limited to: VEGFs, FGF-1, FGF-2, FGF-4, and HGF. Additionally, transcription factors that are associated with up regulating expression of angiogenic cascades may also be transfected into cells used for treatment of lower back pain. Said genes could include: HIF-1, HIF-2, NET (norepinephrine transporter gene), and NF-kB (nuclear factor-kappa B). Antisense oligonucleotides, ribozymes or short interfering RNA (ribonucleic acid) may be transfected into cells for use for treatment of tissue disorders and/or associated pain in order to block expression of antiangiogenic proteins such as IP-10 (Interferon-gamma-inducible 10 kDa protein).

Selection of genes or techniques for introduction of said genes in vivo may be performed in vitro prior to administration so as to allow for methods of screening and selecting the combination that is most angiogenically potent. Testing may be performed by various methodologies known to one skilled in the art. In terms of assessing angiogenic potential, said methodologies include, but are not limited to:

(A) Angiogenic activity may by assessed by the ability to stimulate endothelial cell proliferation in vitro using human umbilical vein endothelial cells (HUVECs) or other endothelial cell populations. Assessment of proliferation may be performed using tritiated thymidine incorporation or by visually counting said proliferating endothelial cells. A viability dye such as MTT or other commercially available indicators may be used.

(B) Angiogenic activity may also be assessed by the ability to support cord formation in subcutaneously implanted matrices. Said matrices, which may include Matrigel® or fibrin gel, are loaded with cells that do not have intrinsic angiogenic potential, for example fibroblasts, transfecting said cells with said genes, and implanting said cells subcutaneously in an animal. Said animal may be an immunodeficient mouse such as a SCID (severe combined immunodeficiency) or nude mouse in order to negate immunological differences. Subsequent to implantation, formation of endothelial cords generated from endogenous host cells may be assessed visually by microscopy. In order to distinguish cells stimulating angiogenesis versus host cells responding to said cells stimulating angiogenesis, a species-specific marker may be used.

(C) Angiogenic activity may be assessed by the ability to accelerate angiogenesis occurring in the embryonic chicken chorioallantoic membrane assay. Cells transfected with angiogenic genes may be implanted directly, or via a matrix, into the chicken chorioallantoic membrane on embryonic day 9 and cultured for a period of approximately 2 days. Visualization of angiogenesis may be performed using in vivo microscopy.

(D) Angiogenic activity may be assessed by the ability to stimulate neovascularization in the hind limb ischemia animal model. In one embodiment, patients diagnosed with hypoxic and/or ischemic disc disease could be treated using gene therapy in a localized manner.

In one embodiment, patients diagnosed with hypoxic and/or ischemic tissue disease could be treated using gene therapy in a localized manner. Specifically, the gene for FGF-1 could be administered in a composition of nucleic acid sequences or one or more triplex DNA compounds, and a nonionic block copolymer. The gene administered could be under control of a strong promoter, for example, the CMV (cytomegalovirus) promoter. The nonionic block copolymer may be CRL-8131 as described in U.S. Pat. No. 6,933,286 (which is incorporated herein by reference in its entirety). Specifically, in such an embodiment 300 milligrams of CRL-8131 may be added to 10 ml of 0.9% NaCl and the mixture solubilized by storage at temperatures of 2-4° C. until a clear solution was formed. An appropriate amount of a FGF-1 expressing plasmid diluted in PBS (phosphate buffered saline) could be added to the mixture and micelles associating the copolymer and the compound could be formed by raising the temperature above 5° C. and allowing the suspension of micelles to equilibrate. The equilibrated suspension would be suitable for administration.

In other embodiments it may be desirable to utilize an angiogenesis-stimulating protein for administration in a therapeutically effective amount. Said protein may be selected from proteins known to stimulate angiogenesis, including but not limited to TPO (thyroid peroxidase), SCF (stem cell factor), IL-1 (interleukin 1), IL-3, IL-6, IL-7, IL-11, flt-3L (fms-like tyrosine kinase 3 ligand), G-CSF (granulocyte-colony stimulating factor), GM-CSF (granulocyte monocyte-colony stimulating factor), Epo (erythropoietin), FGF-1, FGF-2, FGF-4, FGF-5, FGF-20, IGF (insulin-like growth factor), EGF (epidermal growth factor), NGF (nerve growth factor), LIF (leukemia inhibitory factor), PDGF (platelet-derived growth factor), BMPs (bone morphogenetic protein), activin-A, VEGF (vascular endothelial growth factor), VEGF-B, VEGF-C, VEGF-D, P1GF, and HGF (hepatocyte growth factor). In some preferred embodiments, administration of the angiogenesis-stimulating protein is performed by injection directly into a tissue region. In other preferred embodiments, administration of the angiogenesis-stimulating protein can be topical, or various combinations of injected and topical. In some embodiments, the angiogenic-stimulating protein is co-administered with stem or progenitor cells.

### Peripheral Vessel Imaging, Analysis and Treatment

In many instances, a blockage or occlusion of an “upstream” peripheral vessel can significantly reduce the oxygen and/or nutrition flow to the tissues of an extremity or other anatomy supplied by the peripheral vasculature. Similarly, a blockage or occlusion of a “downstream” vessel can significantly degrade the ability of the vascular system to scavenge and/or remove fluids such as blood plasma, cells, various waste products and CO2 from the extremity and/or other vasculature, as well as inhibiting the positive flow of nutrition into relevant tissues-of-interest. Various embodiments of the invention can include imaging of anatomical structures remote from specific skin tissues of interest, with the results of such imaging utilized to detect vascular hypoperfusion, ischemia-associated tissue degradation and/or the need for subsequent treatment including some form of angiogenic stimulation. Various embodiments of the invention disclose novel diagnostic algorithms that can be utilized in the diagnosis and selection of patients for subsequent treatment utilizing pro-angiogenic approaches. Diagnostic imaging algorithms have not been widely use in the treatment of many ischemic-related diseases, since no vascular basis for many degenerative conditions have been accepted in various fields and/or specialties of medicine and surgery. In one aspect of the invention, magnetic resonance angiography (MRA), a special type of MR which creates three-dimensional reconstructions of vessels containing flowing blood, can be utilized to identify vascular abnormalities. For example, by imaging the peripheral vessels, a rating system can be developed measuring the amount of patency of the vessels. The following system is an example of such a system:

Peripheral Vessel Occlusion: Artery and Vein

0 = all extremity vessels are patent
1 = one vessel is stenotic
2 = two vessels are stenotic
3 = one vessel is occluded
4 = one vessel is occluded and one stenotic
5 = two vessels are occluded

Similar to this segmental artery grading system, microvascular perfusion in a targeted skin region could be defined with a numerical scale depending upon the hypoperfusion location in the microvasculature, the quantity of vascular perfusion and the level of potential tissue disruption, damage and/or loss of healing potential (based upon ADC and/or T1ρ.

Integumentary System Perfusion
Possible Classification System

0 = peripheral vasculature and microvasculature provide adequate
perfusion
1a = superficial microvasculature shows “downstream”
hypoperfusion (i.e. venule flow disruption)
1b = superficial microvasculature shows “upstream” hypoperfusion
(i.e. arteriole flow disruption)
1c = superficial microvasculature shows both upstream and
downstream hypoperfusion
2a = deep microvasculature shows “downstream” hypoperfusion
(i.e. venule flow disruption)
2b = deep microvasculature shows “upstream” hypoperfusion (i.e.
arteriole flow disruption)
2c = deep microvasculature shows both upstream and downstream
hypoperfusion
3a = peripheral vasculature shows “downstream” hypoperfusion
(i.e. venous flow disruption)
3b = peripheral vasculature shows “upstream” hypoperfusion (i.e.
arterial flow disruption)
3c = peripheral vasculature shows both upstream and downstream
hypoperfusion
4 = superficial microvasculature region shows no perfusion.
5 = deep microvasculature region shows no perfusion.
6 = peripheral vasculature region shows no perfusion.

This classification system could be as simple as the above chart with complexity being added depending upon various inclusion criteria that could be developed by researching various combinations of imaging techniques as described herein (including, for example, combination imaging strategies, etc.), as well as depending upon the specific anatomy of interest. With further quantitative perfusion research, numerical criteria could determine classification, along with other quantitative imaging assessments already discussed, creating a clinically relevant classification system.

Once a potential region of ischemic circulation and/or microcirculation proximate to a skin location has been identified using the various analysis methods and techniques described herein, various embodiments can include further analysis of anatomical image data of the major circulatory systems that feed into and/or drain out of the microcirculation, to desirably identify any occlusions or partial occlusions in the vasculature and/or microvasculature that may be contributing to the ischemic diagnosis. Where such occlusions or partial occlusions are identified, a desired course of treatment may include angiogenic and/or surgical treatment of the occlusions or partial occlusions alone and/or in combination with angiogenic treatment of the microvasculature proximate to the skin region of interest. Where such occlusions or partial occlusions are not identified, a desired course of treatment may primarily involve angiogenic treatment of the skin region of interest alone.

In various embodiments, combining microvasculature perfusion analysis with imaging and analysis of peripheral artery stenosis and/or the degree of tissue degeneration (and possibly diffusion and/or spectroscopy data) may describe a “new” etiology for subsets of patients with degenerative tissue disease.

In one exemplary embodiment, subjects can be scanned using combinations of Magnetic Resonance Imaging (MRI) and Magnetic Resonance Angiography to (MRA) to assess the condition and/or treatability of their pathology. Exemplary 3D Contrast enhanced MRA scans could be acquired with 50 coronal slices using TR: 5.1 ms, TE: 1.78 ms, voxel size=0.8×0.8×1.5 mm³, SENSE: 4. Data acquired in this method could be assessed and/or combined in various ways. For example, the peripheral vessels on MRA could be graded as occluded, stenotic or open (or other more graduated assessments could be applied). If desired, relevant tissue conditions could be assessed and/or graded. The skin tissue and/or microvascular structure could be analyzed and graded. Image data reflecting the structure and/or perfusion of the capillary vessels and/or microvasculature in various tissues proximate to the tissue of interest could be assessed. in addition, any peripheral branches and/or vessels could be analyzed and graded as occluded, stenotic or open (or other more graduated assessments could be applied), and potentially assessed as to whether they could be sufficient to compensate for an ischemic primary vessel. In addition, MRI and MRA data sets could be overlaid and/or combined to create composite data maps, including the use of color mapping to identify relevant features of interest.

### Imaging of Metabolic Wastes

As previously noted, various embodiments described herein can include the use of imaging and assessment of tissue perfusion combined with measurement and/or assessment of lactate levels within a region of interest with a minimally invasive diagnostic study, which can potentially provide independent confirmation of the disease diagnosis. Removal of waste may be measured by imaging of either lactate or Hydrogen ions over time. If the imaging shows improvement of the amount of these metabolic waste products, then some conclusions can be drawn as to the integrity of the waste removal system. Conversely, an increased level of such wastes could lead to a diagnosis of deficit and/or failing waste removal systems. In addition, real time imaging would be possible with imaging sensitive markers tagged to these, or other waste metabolites.

The diagnosis and relevant treatment of the cause(s) (abnormal load distribution with resultant poor nutrient delivery and waste removal) as described herein could significantly improve clinical management of skin wounds and/or diseases. The ability to measure lactate can provide a metabolic marker that can be utilized to evaluate longitudinally, or eventually, help in the diagnosis of tissue healing. In one exemplary embodiment, MR Proton spectroscopy can be utilized to monitor the lactate content in tissues non-invasively. Alternatively, a MR spectroscopy protocol PRESS (point resolved spectroscopy) with CHESS (chemical shift selective) pulse to suppress water signal could be implemented to quantify lactate content in tissues. This type of spectroscopy in-vivo is possible with specialized hardware (coils) and appropriate software development. Imaging on a subject in a 3T scanner can be accomplished, desirably demonstrating a higher lactate level at more degenerative tissues. As described herein, improved data analysis can occur with PRESS and SHIFT protocols, providing cleaner lactate data.

### Patient Screening

In a variety of cases, patients treated with the various inventions disclosed herein might be refractory to conventional treatments for skin wounds and/or diseases, such as antibiotics, anti-inflammatory medication and/or analgesics. Alternatively, the various treatments described herein may make such conventional treatments more potent and/or effective. In various embodiments, genetic screening and/or whole genome sequencing could be used to elucidate whether a patient that has a greater potential to develop various tissue conditions, as well as to determine which patient may or may not be receptive to various types of gene therapies or other treatments, including angiogenic treatments. Comparing gene sequences in patients with degenerative skin conditions with patients without these disorders can create one or more standards to facilitate a blood test that could alert clinicians to the patient's susceptibility for degenerative tissue disease. This information, coupled with the imaging data already discussed, could refine the decision algorithms for treatment of tissue conditions due to ischemia.

### Dosing

The term “therapeutically effective amount” of a compound is used herein to indicate an amount of an active compound, or pharmaceutical agent, that elicits the biological or medicinal response indicated. This response may occur in a tissue, system, animal or human and includes alleviation of the symptoms of the disease being treated. The exact formulation, route of administration and dosage for the composition and pharmaceutical compositions disclosed herein can be chosen by the individual physician in view of the patient's condition. (See e.g., Fingl et al. 1975, in “The Pharmacological Basis of Therapeutics”, Chapter 1, which is hereby incorporated by reference in its entirety).). Therapeutic treatments can be achieved with small molecule organic drugs or biologics, such as proteins. Typically, the dose range of a small molecule therapeutic agent is administered from about 0.5 to 1000 μg/kg, or 1 to 500 uq/kg, or 10 to 500 μg/kg, or 50 to 100 μg/kg of the patient's body weight per dose. The dose of a therapeutic protein growth factor, such as an FGF, can be administered to the patient topically, intravenously and/or intra-arterially as either a bolus dose or by infusion from about 0.1 to 100 μg/kg of the patient's body weight, or 0.3 to 30 μg/kg, or 1 to 3 μg/kg of the patient's body weight per dose. To achieve localized targeted dosing, FGF-1 can be applied topically to tissue and/or injected either directly into or adjacent to the ischemic tissues and/or their vascular support network, and in various embodiments may be introduced either into or as near as practical to the region of ischemia. Localized dose ranges can be from 10 ng/cm³ to 1 mg/cm³, or 100 ng/cm³ to 100 μg/cm³ or 1 μg/cm³ to 10 μg/cm³ of target tissue per dose. Local doses can be administered at each ischemic tissue location, or where a vessel blockage or occlusion causes significant downstream or upstream effects. The dosage may be a single one or a series of two or more given in the course of one or more days, as is needed by the patient. Where no human dosage is established, a suitable human dosage can be inferred from ED₅₀ or ID₅₀ values, or other appropriate values derived from in vitro or in vivo studies, as qualified by toxicity studies and efficacy studies in animals.

In various embodiments, one or more doses of a therapeutic agent, such as FGF-1, could be injected directly into the ischemic tissues and/or applied adjacent and as closely as possible to the ischemic tissue regions (i.e., via surface and/or subsurface application/injection) using a variety of techniques and/or carriers. One exemplary ideal dose could be determined based on the approximate volume of the ischemic tissues as estimated using MM or other imaging modality. If such imaging or assessment were not practical, a clinician could set a standard dose per ischemic tissue region based on an average skin wound volume or surface area. In various embodiments, an initial dosing goal for FGF-1 could be to achieve a target concentration of 1 to 10 ug of FGF-1 per cm²/cm³ (˜1 ml) of ischemic tissue surface area and/or volume. If the specific tissue volume for a given patient can be determined, this value could be converted into dose levels per ischemic tissue or per cm²/cm³ of ischemic or total tissue area/volume for each individual patient. Alternatively, if an average ischemic tissue volume were determined, a per cm³ dose of such average or actual volume could be used for a patient. In one embodiment, these proposed values could be a dose per treatment day. In other embodiments, efficacy can be improved if weekly or even twice weekly doses were given. For longer term and/or repeated does treatment of patient, the duration of such long term/repeated dosing but could be determined by subsequent MRIs or other imaging of the patient.

Although the exact dosage can be determined on a drug-by-drug basis, in most cases, some generalizations regarding the dosage can be made. The daily small molecule dosage regimen for an adult human patient may be, for example, an oral dose of between 0.1 mg and 500 mg of each active agent, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively, the compositions disclosed herein may be administered topically and/or by continuous intravenous infusion, preferably at a dose of each ingredient up to 400 mg per day. Thus, in various embodiments the total daily dosage by parenteral administration could typically be in a range 0.1 to 400 mg. In some embodiments, the compounds will be administered for a period of continuous therapy, for example for a week or more, or for months or years.

Dosage amount and interval may be adjusted individually to provide a desired plasma levels of the active moiety (which can include a zero or negligible plasma volume of the active moiety), which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC (high-performance liquid chromatography) assays or bioassays can be used to determine plasma concentrations.

Dosage intervals can also be determined using MEC value. Compositions could be administered using a regimen which maintains plasma levels approximate to zero, as well as plasma levels above the MEC for 10-90% of the time, between 30-90% and between 50-90%.

The amount of a given composition administered will, of course, be dependent on the subject being treated, on the subject's weight, the severity of the affliction, the manner of administration and the judgment of the prescribing physician.

### Surgical Tools, Procedures and Techniques

In many situations, especially advanced cases involving significant damage to and/or infection of sub-fascial tissues and/or bone, surgical interventions may be required. Once a targeted anatomical region and intended treatment regimen have been determined and where subsurface introduction of an angiogenic substance may be desirous, a surgical access path and procedure will typically be determined. In many cases, the simple injection of drugs, proteins, cells and/or compounds into the vasculature and/or soft tissues can be accomplished using hypodermic needles, catheters and/or other minimally- or less-invasive surgical devices. However, where such injections desirably target specific tissues, where such devices may be utilized proximate to sensitive and/or fragile tissues structures, where such devices must transition through and/or into denser or harder tissues, or where a more invasive surgical intervention is desired, additional surgical techniques and/or tools may be required.

In many cases, minimally-invasive devices such as hypodermic needles and cannulae can be introduced via a needle-stick or small incision in the patient's skin and soft tissues, and guided to a desired location within the anatomy using fluoroscopic or other non-invasive types of visualization. For example, if minimally-invasive access proximate to a vascular narrowing or blockage is desired, a non-invasive view of the vessel of interest (and surrounding anatomy) may be taking using a fluoroscopic visualization system such as a C-arm, commercially available from GE Medical Systems. The vessel could be visualized on the scan (which may include the use of contrast agent), and the needle tip could be inserted through the patient's skin and soft tissues and advanced until it is proximate to the desired tissue structure(s). It is possible that intraoperative CT, MRI or ultrasound (or other imaging modalities not yet in clinical use) may be used by the surgeon to ascertain, to a greater degree of clarity, the exact position of the device and/or verify the location of delivery of the active drug and/or carrier. If the carrier is not radiopaque, then a sufficient amount of a radiopaque material, such as barium powder, may be mixed with the carrier, angiogenic material and/or other injectable compound to allow fluoroscopic visualization and localization of the compound.

In various embodiments described herein, it may be desirous to inject compositions and/or materials, including angiogenic compounds, into specific and/or discrete locations within a patient's anatomy. For example, where imaging, analysis and diagnosis indicates a hypoperfused capillary bed proximate to a skin wound, it may be desirous to inject an angiogenic factor into and/or near the capillary bed in an attempt to produce angiogenesis within the localized region. Depending upon the clinical needs, the injection may simply be into the dermal and/or sub-dermal tissues, or the injection may desirably be proximate to a specific area of the vascular supply to the capillary bed (i.e., proximate to a vessel constriction and/or obstruction that may be remote from the capillary bed).

If desired, a method of treating a vascular deficiency could include the mechanical creation of a channel or path within various tissues of the patient's body using a hypodermic needle or other device. Once the needle has been advanced along a path, the needle may be withdrawn while concurrently injecting periodic “bursts” (i.e., boluses) or a continuous “string” or strings of an angiogenic compound into the path evacuated by the needle. This path may be continuous or intermittent, as desired, and desirably the compound left behind within the path will induce the eventual creation of a new vascular path (or portions thereof) along the needle track.

### Homing Receptors and Targeted Drug/Cell Delivery

In various embodiments, angiogenic treatments such as those described herein could further benefit from their employment with “homing receptors” and/or other targeted drug/cell delivery techniques that desirably increase and/or maintain a desired concentration of an angiogenic factor (and any associated medications/tissues) in some parts of the body relative to others. In various embodiments, an appropriate targeted delivery system could be utilized to deliver a certain amount of the desired therapeutic agent to a targeted tissue and/or treatment area within the body, which may include such delivery for a prolonged period of time. Such an approach would desirably maintain a required plasma and/or tissue level of the therapeutic agent in the body, without damage and/or significant unwanted effect to other healthy tissues. In various preferred embodiments, such targeted delivery systems could allow for injection and/or ingestion by a patient of the therapeutic agent, which could then desirably concentrate the agent in the desired tissue and/or tissues, without unwanted and/or unhealthy concentrations of the agents outside of the targeted tissue(s).

There are a variety of drug delivery vehicles that could be utilized in conjunction with various treatment described herein, including polymeric micelles, liposomes, lipoprotein-based drug carriers, nano-particle drug carriers and/or dendrimers (as well as many others). A desirable drug delivery vehicle is non-toxic, biocompatible, non-immunogenic, biodegradable, and/or will desirably avoid recognition and/or attack by the patient's defense mechanisms.

For example, a desired vehicle for targeted drug delivery could be the liposome, which is non-toxic, non-hemolytic, and non-immunogenic (even upon repeated injections), is biocompatible, biodegradable and can be designed to avoid clearance mechanisms (i.e., reticuloendothelial system—RES, renal clearance, chemical or enzymatic inactivation, etc.). Lipid-based, ligand-coated nanocarriers can store their payload in the hydrophobic shell or the hydrophilic interior depending on the nature of the drug being carried. To combat the relatively low stability of liposomes in vitro, polyethylene glycol (PEG) can be added to the surface of the liposome, and by increasing the mole percent of PEG on the surface of a liposome by 4-10%, significantly increased circulation time in vivo (from 200 to 1000 minutes) can be achieved.

As another example, polymeric micelles could be used to carry therapeutic agents, including agents which may have poor solubility. Polymeric micells can be prepared from certain amphiphilic co-polymers consisting of both hydrophilic and hydrophobic monomer units. Similarly, dendrimers (also polymer-based delivery vehicles) could be utilized.

In other embodiment, a biodegradable particle could be utilized to target diseased tissue as well as deliver a therapeutic agent payload as a targeted and/or controlled-release therapy. Biodegradable particles bearing ligands to P-selectin, endothelial selectin (E-selectin) and ICAM-1 can adhere to inflamed endothelium, which could allow their use for targeting and/or treating cardiac tissue and/or other tissue structures.

In various embodiments, artificially designed nanostructures constructed out of nucleic acids such as DNA could be utilized for targeted delivery, which may further incorporate a DNA-based computing system (i.e., artificial nucleic acid nanodevices) that enables targeted drug delivery to a desired tissue or tissues based upon directly sensing its surrounding environment. Such devices could make use of DNA solely as a structural material and/or a chemical constituent, and would not necessarily seek to use the DNA's biological role as the carrier of genetic information. Nucleic acid logic circuits could potentially be incorporated in a system that releases a therapeutic drug (and/or one of more of a plurality of drugs contained in the delivery vehicle) in response to a stimulus, such as a specific detected mRNA. Alternatively, a DNA “box” of other similar structure could incorporate a controllable “lid” or opening (i.e., synthesized using the DNA origami method) which desirably encapsulates a therapeutic agent in its closed state, and then opens to release the agent in response to a desired external stimulus.

### Imaging and Treatment of Poorly Vascularized Tissues

The various embodiments described herein could also have significant utility for the imaging, assessment and/or treatment of a variety of conditions within poorly and/or less vascularized tissues in mammals. Some examples of such tissues can include the tympanic membrane of the ear, the vocal folds of the larynx, various synovial membranes of the body (i.e., articular, vesicular and/or vaginal), some eye tissues and/or other bodily tissues. In many instances, tissues that are normally supplied by lesser blood flows and/or less extensive vascular networks will rely primarily on diffusion and/or lymphatic flow for cellular oxygen, nutrition and/or waste removal. In many cases, these tissues are slow to repair following an injury, and degenerative conditions of the vasculature (i.e., atherosclerosis, for example), can disproportionately affect the limited vascular networks supporting these tissues.

FIG. 20 depicts a lateral aspect of a tympanic membrane (e.g., eardrum, tympanum) of a human ear. The tympanic membrane separates the tympanic cavity from the external acoustic meatus, and it collects sound energy to transfer it to the small bones in the middle ear. It is a thin and tense semitransparent membrane, is nearly oval in form, and is directed very obliquely downward and inward.

The tympanic membrane of the ear is a three-layer structure, typically nine to ten millimeters in size. The outer and inner layers of the membrane consist of epithelium cells, with squamous epithelium laterally and respiratory mucosa medially, with a fibrous layer between. When inspected through an otoscope, normally it has a pearly-grey, semi-transparent appearance. The outer margin of the eardrum is thickened and forms a fibro-cartilaginous ring, and fixed in the tympanic sulcus. The upper fifth of the eardrum is slack that called the pars flaccida, and the lower four-fifths is called the pars tensa.

The blood supply of the tympanic membrane comes from the ear canal superiorly, and is derived from both the circumferential branch 200 and the manubrial branch 210 of the deep auricular branch of the maxillary artery 230. The branches arise from the deep auricular branch of the maxillary artery to the outer surface, while the inner surface of the membrane is supplied by the stylomastoid branch of the occipital, and the tympanic branch of the maxillary artery (via various radial supply branches 240).

The tympanic membrane receives its main nerve supply from the auriculotemporal branch of the mandibular nerve. The auricular branch of the vagus, and the tympanic branch of the glossopharyngeal also supply it.

When the tympanic membrane is damaged, its ability to collect and transfer sound energy is often reduced and/or eliminated. In general, perforations to the tympanic membrane can occur as a result of defects in the middle layer (which contains elastic collagen fibers) or as a result of trauma, such as an object in the ear, a slap on the ear, or an explosion or other pressure wave. While the vast majority of minor eardrum damage can heal naturally within a period of three or more months, major eardrum damage and/or slow healing wounds may require supplemental treatments and/or surgery such as tympanoplasty (i.e., surgery performed to reconstruct a perforated tympanic membrane or the small bones of the middle ear).

The purpose of tympanoplasty is to repair the perforated eardrum, and sometimes the middle ear bones (ossicles) that consist of the incus, malleus, and stapes. In various surgeries, tympanic membrane grafting may be required. If needed, grafts are usually taken from a vein or fascia (muscle sheath) tissue on the lobe of the ear. Synthetic materials may be used if patients have had previous surgeries and have limited graft availability. There are various grades of tympanoplasty:

Type I tympanoplasty is called myringoplasty, which only involves the restoration of the perforated eardrum by grafting.

Type II tympanoplasty, which is used for tympanic membrane perforations with erosion of the malleus. It involves grafting onto the incus or the remains of the malleus.

Type III tympanoplasty, which is indicated for destruction of two ossicles, with the stapes still intact and mobile. It involves placing a graft onto the stapes, and providing protection for the assembly.

Type IV tympanoplasty, which is used for ossicular destruction, which includes all or part of the stapes arch. It involves placing a graft onto or around a mobile stapes footplate.

Type V tympanoplasty, which is used when the footplate of the stapes is fixed.

Depending on its type, tympanoplasty can be performed under local or general anesthesia. In small perforations of the eardrum, type I tympanoplasty can be easily performed under local anesthesia with intravenous sedation. An incision is made into the ear canal and the remaining eardrum is elevated away from the bony ear canal, and lifted forward. The surgeon can utilize an operating microscope to enlarge the view of the ear structures. If the perforation is very large or the hole is far forward and away from the view of the surgeon, it may be necessary to perform an incision behind the ear. This elevates the entire outer ear forward, providing access to the perforation.

Once the hole is fully exposed, the perforated remnant is rotated forward, and the bones of hearing are inspected. If scar tissue is present, it can be removed either with micro hooks or by use of a laser. If necessary, graft tissue is then taken either from the back of the ear, the tragus (small cartilaginous lobe of skin in front the ear), or from a vein. The tissues are thinned and dried. An absorbable gelatin sponge may by placed under the eardrum to support the graft. The graft is then inserted underneath the remaining eardrum remnant, which is folded back onto the perforation to provide closure. Very thin sheeting is usually placed against the top of the graft to prevent it from sliding out of the ear when the patient sneezes. If the ear was opened from behind, the ear is then stitched together. Usually, the stitches are buried in the skin and do not have to be removed later. A sterile patch is placed on the outside of the ear canal and the patient returns to recovery.

In many instances, the employment of angiogenic substances and related techniques, such as those described herein, can be extremely useful in the treatment of minor and/or major damage to the tympanic membrane. Because the tympanic membrane is very thin, it can often be difficult to image and/or visually identify tears and/or perforation in the membrane tissues. Moreover, it may be difficult to manipulate and/or suture the membrane tissues, which can tear easily. Moreover, because the tympanic membrane is “poorly” vascularized, a small tear or perforation can often interrupt the limited vascular flow for a large region of the membrane, further delaying and/or preventing the nature healing responses.

In at least one exemplary embodiment, a small tissue graft or “patch” (which may be natural and/or artificial tissue and/or other materials, including resorbable or non-resorbable materials), can be impregnated with angiogenic compounds and placed in contact with torn or perforated tympanic membrane tissues, which will desirably (1) maintain the torn edges of the membrane in a close proximity to one another, (2) induce a healing response within the surface and/or subsurface membrane tissues and/or (3) induce an angiogenic response in the tissues of the membrane to facilitate wound healing in a timely manner. In various alternative embodiments, a topical compound comprising an angiogenic factor (i.e., a liquid, powder or gel-like compound) can be applied to the exterior and/or interior surface of the torn or perforated membrane to desirably induce a healing response, such as described herein, which could alternately include the use of aerosolized and/or “spray-type” products for application directly to the membrane.

In another example of wound healing, the vocal folds located within the larynx (at the top of the trachea) could be treated in a similar manner using an angiogenic compound. The vocal folds are attached posteriorly to the arytenoid cartilages, and anteriorly to the thyroid cartilage. They are part of the glottis which includes the rima glottidis. Their outer edges are attached to muscle in the larynx while their inner edges, or margins are free, forming the opening called the rima glottidis. They are constructed from epithelium, but they have a few muscle fibers in them, namely the vocalis muscle which tightens the front part of the ligament near to the thyroid cartilage. The vocal folds are flat triangular bands and are pearly white in color. Above both sides of the glottis are the two vestibular folds or false vocal folds which have a small sac between them. Situated above the larynx, the epiglottis acts as a flap which closes off the trachea during the act of swallowing to direct food into the esophagus. If food or liquid does enter the trachea and contacts the vocal folds it causes a cough reflex to expel the matter in order to prevent pulmonary aspiration.

Males and females have different vocal fold sizes. Adult male voices are usually lower pitched due to longer and thicker folds. The male vocal folds are between 1.75 cm and 2.5 cm in length, while female vocal folds are between 1.25 cm and 1.75 cm in length. The vocal cords of children are much shorter than those of adult males and females. The difference in vocal fold length and thickness between males and females causes a difference in vocal pitch.

Mature human vocal folds are composed of layered structures which are quite different at the histological level. The topmost layer comprises stratified squamous epithelium which is bordered by ciliated pseudostratified epithelium. The inner lining surface of this squamous epithelium is covered by a layer of mucus (acting as a mucociliary clearance), which is composed of two layers: a mucinous layer and serous layer. Both mucus layers provide viscous and watery environment for cilia beating posteriorally and superiorly. The mucociliary clearance keeps the vocal folds essentially moist and lubricated. The epidermis layer is secured to the deeper connective tissue by basement membrane. Due to the primarily amorphous fibrous and nonfibrous proteins in the lamina propria, the basement membrane applies strong anchoring filaments like collagen IV and VII to secure the hemidesmosome of basal cells to the lamina propria. These attachments are strong enough to sustain beating and stretch, to which vocal folds are normally subjected. The population density of some of the anchoring fibers in the basement membrane, such as collagen VII, is genetically determined, and these genetics may influence the health and pathogenesis of the vocal folds.

Vocal fold injuries can have a number of causes including chronic overuse, chemical, thermal and mechanical trauma such as smoking, laryngeal cancer, and surgery. Other benign pathological phenomena like polyps, vocal fold nodules and edema can also introduce disordered phonation. Injuries to human vocal folds typically elicits a wound healing process characterized by disorganized collagen deposition and, eventually, formation of scar tissue. In the proliferative phase of vocal fold wound healing, if the production of HA and collagen is not balanced (which means the HA level is lower than normal), the fibrosis of collagen cannot be regulated. Consequently, regenerative-type wound healing often turns to be the formation of scar. Scarring may lead to the deformity of vocal fold edge, the disruption of LPs viscosity and stiffness. Patients suffering from vocal fold scar always complain about increased phonatory effort, vocal fatigue, breathlessness, dysphonia as well. Vocal fold scar is one of the most challenging problems for otolaryngologists because it's hard to be diagnosed at germinal stage and the function necessity of vocal folds is delicate.

In at least one exemplary embodiment, vocal fold injuries can be treated by application of a small tissue graft or patch impregnated with angiogenic compounds and placed in contact with diseased, damaged, torn or perforated vocal fold tissues, which will desirably (1) maintain the torn edges of the tissues in a close proximity to one another, (2) induce a healing response within the surface and/or subsurface vocal fold tissues and/or (3) induce an angiogenic response in the vocal fold tissues to facilitate wound healing in a timely manner. In various alternative embodiments, a topical compound comprising an angiogenic factor (i.e., a liquid, powder or gel-like compound) can be applied to the exterior surface of the damaged vocal fold to desirably induce a healing response, such as described herein. If desired, additional internal tissue treatments involving angiogenic compounds could be injected into one or both of the vocal folds, either alone or in combination with the various topical treatments described herein.

Burns and/or Other Skin Wounds

Various embodiments described herein could also have particular utility with regards to various types of damaged and/or injured surface and/or subsurface skin tissues, including surface/subsurface skin tissue burns due to excessive heat, excessive cold, chemical contact, radiation effects, wind abrasion and/or otherwise induced tissue damage. It should be understood that the various imaging, diagnosis, assessment and/or treatment modalities described herein could be utilized in conjunction with the treatment and/or management of such wounds, including various combinations of the various embodiments disclosed herein.

### Exemplary Treatments

Ex. 1—Foot Ulcer Treatment

In one exemplary embodiment, a patient with a foot ulcer or other similar anatomical issues, who has not improved with conservative care, can undergo perfusion imaging as described herein that, when analyzed, demonstrates one or more areas of ischemia proximate to the ulcerous skin tissue. Further imaging studies could be obtained to analyze the vascular supply in the extremity in detail and identify specifics as to the anterior, posterior, cephalad, caudad, medial/lateral and/or left/right location of the perfusion deficits. One or more tissue perfusion 2D or 3D maps (which could include structural and/or colorized flow maps) could be generated for further detail. Maps prepared using different imaging modalities (i.e., MRA and MRI, for example) or identifying different anatomical characteristics (i.e., images reflecting perfusive flow overlain by images reflecting soft tissue and/or bone structures and/or metabolic waste imaging) could be compared and/or overlain, and the resulting data tabulated and/or analyzed. The physician and/or surgeon could begin planning the proper placement of the angiogenic factor by topical application and/or injection, as well as with associated prosthesis, delivery vehicles and/or therapeutic compounds. The angiogenic factor could be FGF-1 or FGF-1 mutant or other angiogenic factors. The angiogenic factor may be formulated in a variety of vehicles and/or carriers defined for specific surgical needs.

As an example, the foot ulcer may require an angiogenic factor in an externally placed vehicle or prosthesis or alternatively in a vehicle that requires an anchor or some other attachment device that would allow a broad and stable surface area for delivery of the drug. Various other modifications may be required depending upon the location and/or use of the skin tissue surface (i.e., is the surface on the bottom of the foot or in a load-bearing region). In addition, the location of the damaged tissues may require specific angiogenic formulations, vehicles, matrixes, synthetics, carriers, mutants, attachments, anchors, dosages, repeat doses, delivery devices, image guided delivery and/or targeted delivery selections. In addition, if a portion of the tissue requires replacement and/or was sacrificed as part of the normal treatment or approach to gain access to the drug delivery zone and a reconstruction was required or desired, a tissue graft might be performed at the same time as the angiogenic treatment or in a staged procedure. In addition, if a preoperative defect would require reconstruction prior to the angiogenic treatment, then the reconstruction and/or grafting procedure could be done first and the angiogenesis performed at the same time or in a second stage.

If other regenerative therapy is planned, either tissue based, cell based, gene based or protein based, or some other biologic or synthetic regenerative or tissue engineering treatment, and it was ascertained that the above diagnostic and angiogenic treatment and/or tissue surface reconstruction was desired prior to or during the regenerative treatment, then the above diagnostic and treatment protocol could be performed in concert with the regenerative treatment or in a staged fashion.

To monitor the amount of stress that damaged skin region experiences and thus guide postoperative wound load bearing, micro force transducers or other devices could be positioned in strategic areas to measure the amount, location and distribution of the stresses at the wound and/or adjacent anatomical regions. These force transducers could be linked with portable electronic devices (i.e., “smart” phones or other devices) as well as other wearable or implantable monitoring devices that could include accelerometers, GPS and strain gauges and/or other micro mechanical and biologically compatible instruments. These may be manufactured with either synthetic or biologic material, or combinations thereof. If desired, the portable electronic device could include a software application or other feature that interpreted data from the force transducers to provide “overload” warnings and/or warnings that a patient was not complying with some aspect of the treatment protocol (i.e., not wearing the prosthesis and/or required offloading device when the patient and phone move a certain distance away from the prosthesis/offloading device).

As previously noted, the amount of stress, loading and/or movement the skin wound might be subjected to could be modified by the offloading device, and in various embodiments such devices could be modifiable in the amount of “load sharing” and/or movement they allow, if desired. In a manner similar to a crutch used by a patient after orthopedic surgery, the patient may undergo progressively increased amounts of weight bearing following the ulcer treatment and/or reconstructive procedure, including the application of progressive, monitored, measurable, controllable stress that could provide the correct signal for optimal vessel growth and/or tissue matrix repair.

Ex. 2—Peripheral Artery Analysis Combined with Microvascular Dynamic Perfusion

In various embodiments, the arterial tree and body blood flow can be simultaneously and/or sequentially evaluated in an extremity or other anatomical region for the purpose of vascular mapping of the extremity or other region of interest. The goal of such a study can be (1) to develop a safe and reproducible technique of MRA and perfusion utilizing one injection of contrast, (2) to measure extremity perfusion and compare intra-subject and inter-subject results with the degree of peripheral artery stenosis and microvascular compromise, (3) to begin evaluating normal controls, and/or (4) to diagnose and/or treat the patient.

In one exemplary embodiment, both MRA and dynamic perfusion imaging can be performed with contrast enhancement. Subject images can be acquired with a Philips Achieva 3T system. For all imaging protocols, a 330 mm*300 mm FOV and a 6-element SENSE torso RF coil can be used. The imaging session can start with the perfusion scan following the standard calibration scans. A 3D FFE sequence with TR/TE=3.5 ms/1.5 ms, SENSE factor: 2.5(AP), 2(RL), flip angle=30°, with dynamic scan time of 2.9 seconds can be used and 7 or more slices in sagittal orientation with 6 mm thickness and 1.9 mm*1.9 mm pixel size can be acquired. In one example a total of 114 volumes could be collected, with 2 or more of them before contrast injection. After the dynamic scans, T1 weighted anatomical images in sagittal plane can be collected using a TSE sequence with 0.5*0.5*3 mm³ voxel size. Fourteen slices might cover the same volume as dynamic scans. TR/TE=900 ms/10 ms, flip angle=90°. This can be followed by a T2 weighted scan that has identical geometry to the T1 scans and TR/TE=2940 ms/120 ms, flip angle=90°. Finally, contrast enhanced angiography scans can be collected. Contrast bolus arrival can be observed real-time using a single, 50 mm thick coronal slice using FFE sequence in dynamic mode, collecting images every 0.5 s. Once the contrast arrives in a target vessel, actual 3D angiography scan should be started by the operator immediately. In one example, TR/TE=5.1 ms/1.78 ms, voxel size=0.8*0.8*1.5 mm³, with SENSE factor=4 can be used to acquire 50 coronal slices.

Peripheral vessels on MRA can be graded as occluded, stenotic or open. Region of interest (ROI)-averaged time course (from whole extremity and/or localized tissue regions) can be converted into a fractional enhancement time course and analyzed using a compartmental or other model (Larsson, et. al. MRM 35:716-726, 1996; Workie, et. al. MRI, 1201-1210, 2004). In one tissue modeling embodiment, the model fitting can result in 6 parameters: K^{trans}′ (apparent volume transfer constant), kₑₚ (rate constant), Vp! (apparent fractional plasma volume), E (extraction fraction), tlag (arrival time of tracer in the ROI) and baseline.

Subjects may demonstrate one or more peripheral vessel as normal, occluded or stenotic. Subjects may further demonstrate one or more areas of microvascular compromise, which can similarly be rated as normal, occluded or stenotic. Subjects in need of angiogenic treatment may demonstrating an order of magnitude lower value of perfusion and/or microperfusion, indicating a perfusion abnormality beyond any MRA identified lesions. A variety of other perfusion parameters (kₑₚ, Vp and E) can be extracted from the acquired data and are helpful in the interpretation. Pixel by pixel images can be generated of any parameter (and through any slice) for visual comparison.

Color coded scans and/or color maps can conveniently and accurately demonstrate the disease visually and is more adaptable for clinical use (although non-color and other data sets and maps can be used, if desired). Using this technique, data can be entered into a pooled multicenter database. Subsets of patients that may have a significant vascular and resultant ischemic/hypoxic component to their disease can then be identified.

Various methods for studying the vascular anatomy and dynamics of various skin tissue regions in one scanning session using a contrast agent is demonstrated. Skin and related tissue anatomy, vascular anatomy and sophisticated perfusion data can be obtained. For example, Kₜᵣₐₙₛ can represent the rate of transfer of contrast delivered to the interstitial tissue, while the kₑₚ is the rate the delivered contrast is cleared from the interstitial tissue, or “wash out”. In addition, E (the extraction fraction of contrast during its initial passage within a given volume [ROI]) is another helpful parameter. If decreased blood supply is an etiologic factor in a patient subset, this technique provides a mechanism by which investigators can study this disease in vivo.

Newer MR techniques such as MR Spectroscopy can be added to identify metabolic abnormalities within various tissues. For example, lactate, an end product of anaerobic metabolism, may be increased in tissues that obtain their nutrients from microvasculature with poor perfusion.

Ex. 3—DCE-MRI and Vessel Perfusion

In another exemplary embodiment, DCE-MRI could be performed as the last scan in a given imaging session. One exemplary protocol based on a 3D gradient-echo sequence could employ the following parameters: TR=3.4 ms, TE=1.2 ms, Flip-angle=30°, NEX=1, and 36.4 sec. temporal resolution.

Any number of dynamic frames could be taken. For example, 22 dynamic frames may be prescribed, with a contrast agent administered manually as a bolus w/a saline flush via a vein at the onset of the 3^{rd} dynamic frame. The overall injection time of both the contrast and saline can be less than 10 seconds. Various contrast agents may be used, including 0.1 mmol/kg of Gadopentetic acid or Magnevist commercially available from Bayer Schering Pharma of Berlin-Wedding, Germany if desired, an identical single-frame image could be acquired 20 or more minutes later to observe any delayed gadolinium enhancement in various tissues.

The generation of a contrast-induced signal enhancement map (SE-map) of the relevant data and a subsequent analyses can be performed. If desired, the contrast-induced signal enhancement in DCEMRI can be normalized into percentage enhancement by first subtracting the baseline (which can be the mean of 2 pre-contrast dynamic frames) from all subsequent post-contrast time frames (i.e., from the 3^{rd} to the last dynamic frames) and then dividing the differences by the baseline. This operation can be carried out either in a pixel-by-pixel basis for creation of an enhancement map or in a region-of-interest (ROI)-averaged sense for enhancement time-course. The T2 scan can be used to indicate the area analyzed by the pixel-by-pixel created color enhancement map of the tissue perfusion. A graph could show time course data from ROI's. Rectangles placed on various tissue structures could represent ROI's drawn and/or derived (i.e., by a computer modeling program).

Various aspects of the data can be examined, either alone or in various combinations, including spatial maps of signal enhancement at one or more fixed time points and an ROI-averaged temporal characteristic in the time course data. Spatial mapping can yield results and/or quantities reflecting an effective capillary perfusion.

Other parameters derived from the temporal characteristic can provide complementary information regarding changes in the capillary structure. For the temporal analysis, the volume-averaged signal enhancement time course can be generated. The enhancement time course can be initially analyzed in a semi-quantitative manner, assessing the parameters such as the maximum enhancement value (%), the time-to-peak (sec), and the clearance rate (%/sec), which in this example could be defined as the slope of the straight line between the 4^{th} and the last (22^{nd}) frame. Other quantitative analyses based on a compartmental model, shape-based fitting and/or nonlinear pharmacokinetic models could be utilized.

### Other Joints, Organs and Tissues

The various embodiments described herein, including the analysis of image data, diagnosis of ischemic disease and treatments thereof using various tools, techniques and surgical methods can be applied to various other tissues in a human or animal body, including any soft or hard tissues including, without limitation, joint tissues, a spine, an elbow, a shoulder, a wrist, a hand, a finger, a jaw, a hip, a knee, an ankle, a foot, or a toe joint. In a similar manner, various alternative embodiments and/or modifications thereof could be used for the imaging, analysis, diagnosis and/or treatment of soft tissue structures and/or other organs, including the heart, heart tissue grafts and/or heart transplants.

In various alternative exemplary embodiments, methods of diagnosing a condition responsible for degenerative joint conditions could include one or more of the following steps:

a) assessing a patient by one or more of the following steps:
(i) obtaining image data of one or more joint structures of the patient;
(ii) identifying one or more regions of interest within the image data;
(iii) analyzing the one or more regions of interest to identify one or
more areas of intraosseous hypoperfusion proximate to one or
more areas of osteochondral tissues of the joint; and
(iv) diagnosing the patient with said intraosseous hypoperfusion
proximate to said osteochondral tissue of the joint.

In various alternative exemplary embodiments, methods of diagnosing a condition responsible for degenerative tissue conditions could include one or more of the following steps:

a) assessing a patient by one or more of the following steps:
(i) obtaining image data of one or more tissue structures of the patient;
(ii) identifying one or more regions of interest within the image data;
(iii) analyzing the one or more regions of interest to identify one or
more areas of hypoperfusion within the tissue structures; and
(iv) diagnosing the patient with said hypoperfusion within the tissue
structures of the patient.

Of course, once a candidate is identified using one or more of these methods, a suitable treatment regime can be performed on the patient, such as the various treatments described herein.

### Headings

The headings provided herein are merely for the reader's convenience, and should not be construed as limiting the scope of the various disclosures or sections thereunder, nor should they preclude the application of such disclosures to various other embodiments or sections described herein.

### Incorporation by Reference

The entire disclosure of each of the publications, patent documents, and other references referred to herein is incorporated herein by reference in its entirety for all purposes to the same extent as if each individual source were individually denoted as being incorporated by reference.

### Equivalents

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention. The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the claims provided herein.

What is claimed is:

1. A method for promoting angiogenesis within an ischemic subsurface tissue site, comprising:
selecting a patient in need of increased blood flow in the ischemic subsurface tissue;
topically applying to a surface tissue site adjacent to the ischemic subsurface tissue site an effective amount of a topical compound comprising FGF-1, wherein the effective amount of the topical compound promotes angiogenesis within at least a portion of the ischemic subsurface tissue.

2. The method of claim 1, wherein the surface tissue site comprises a skin ulcer.

3. The method of claim 1, wherein the surface tissue site comprises a compression ulcer.

4. The method of claim 1, wherein the surface tissue site comprises a diabetic foot ulcer.

5. The method of claim 1, wherein the surface tissue site comprises a healthy tissue site adjacent to a skin ulcer.

6. The method of claim 1, wherein the surface tissue site comprises a burn site.

7. The method of claim 1, wherein the step of topically applying to a surface tissue site adjacent to the ischemic subsurface tissue site an effective amount of a topical compound comprises placing a wound dressing impregnated with FGF-1 into intimate contact with the surface tissue site adjacent to the ischemic subsurface tissue site.

8. The method of claim 1, wherein the step of topically applying to a surface tissue site adjacent to the ischemic subsurface tissue site an effective amount of a topical compound comprises placing a wound dressing into intimate contact with the surface tissue site adjacent to the ischemic subsurface tissue site, the wound dressing comprising FGF-1.

9. The method of claim 1, wherein the step of topically applying to a surface tissue site adjacent to the ischemic subsurface tissue site an effective amount of a topical compound comprises placing a tissue graft material into intimate contact with the surface tissue site adjacent to the ischemic subsurface tissue site, the tissue graft material comprising FGF-1.

10. A method for promoting angiogenesis within a subsurface tissue site proximate to a skin ulcer of a patient, comprising:
topically applying to an external surface of the skin ulcer an effective amount of a topical compound comprising FGF-1, wherein the effective amount of the topical compound promotes angiogenesis within the subsurface tissue site.

11. The method of claim 10, wherein the step of topically applying to an external surface of the skin ulcer an effective amount of a topical compound comprising FGF-1 comprises topically applying to an external skin surface of the patient an effective amount of a topical compound comprising FGF-1, the external skin surface of the patient comprising the external surface of the skin ulcer and at least a portion of healthy skin tissue proximate to the skin ulcer.

15. A method of treating a skin wound of a patient comprising:
topically applying to a surface tissue of the skin wound an effective amount of a topical compound comprising FGF-1, wherein the effective amount of the topical compound induces cell growth or repair within at least one member of the group consisting of dermal fibroblasts and epidermal keratinocytes.

16. The method of claim 15, wherein the effective amount of the topical compound further induces cell growth in vascular endothelial cells and the formation of new blood vessels in an ischemic subsurface tissue layer underlying the surface tissue.

17. The method of claim 15, wherein the skin wound is a diabetic ulcer of the patient.

18. The method of claim 15, wherein the skin wound is a pressure ulcer of the patient.

19. The method of claim 15, wherein the topical compound further comprises an anti-inflammatory agent.

20. The method of claim 15, wherein the topical compound further comprises an antibiotic.
